(19) United States
(12) Patent Application Publication
(10) Pub. No.: US 2025/0098534 A1
(43) Pub. Date: Mar. 20, 2025

(54) LIGHT RECEIVING ELEMENT, ELECTRONIC DEVICE, AND POLYCYCLIC COMPOUND FOR LIGHT RECEIVING ELEMENT

(71) Applicant: Samsung Display Co., Ltd., Yongin-si (KR)

(72) Inventors: HYEJIN JUNG, Yongin-si (KR); Hwa Sook RYU, Yongin-si (KR); SEOKGYU YOON, Yongin-si (KR)

(21) Appl. No.: 18/746,822

(22) Filed: Jun. 18, 2024

(30) Foreign Application Priority Data
Sep. 12, 2023 (KR) 10-2023-0120772

(51) Int. Cl. H10K 85/60; C07D 495/04; H10K 30/40; H10K 65/00

(52) U.S. Cl. CPC H10K 85/6576; C07D 495/04; H10K 30/40; H10K 85/621; H10K 85/653; H10K 85/654; H10K 85/655; H10K 85/657; H10K 85/6572; H10K 65/00

(57) ABSTRACT

A light receiving element includes a first electrode, a second electrode on the first electrode, and a light receiving layer between the first electrode and the second electrode and including a polycyclic compound represented by Formula 1.

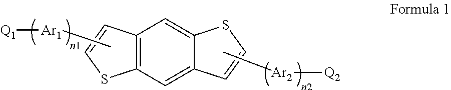

### CROSS-REFERENCE TO RELATED APPLICATION

The present application claims priority to and the benefit of Korean Patent Application No. 10-2023-0120772, filed on Sep. 12, 2023, in the Korean Intellectual Property Office, the entire content of which is incorporated herein by reference.

### BACKGROUND

### 1. Field

One or more embodiments of the present disclosure relate to a light receiving element, an electronic device, and a polycyclic compound for the light receiving element.

### 2. Description of Related Art

Various types (kinds) of electronic devices are utilized to provide image information, and the electronic devices serve one or more suitable functions that allow electronic and organic communication with users, such as detecting user inputs. For example, current electronic devices include a function for detecting and recognizing external inputs provided from the outside.

External input recognition methods include a capacitance method detecting changes in capacitance formed between electrodes, an optical method detecting incident light utilizing an optical sensor, and an ultrasonic method detecting vibration utilizing a piezoelectric material. Meanwhile, when the optical sensor is included and utilized, what is needed and desired is to increase efficiency in absorbing light and converting absorbed light into electrical signals to improve sensitivity of light receiving elements of the optical sensor.

### SUMMARY

One or more aspects of embodiments of the present disclosure are directed toward a light receiving element having improved light receiving efficiency and improved deposition quality.

One or more aspects of embodiments of the present disclosure are directed toward an electronic device having improved light receiving efficiency.

One or more aspects of embodiments of the present disclosure are directed toward a polycyclic compound capable of improving the light receiving efficiency and deposition quality of a light receiving element.

Additional aspects will be set forth in part in the description which follows and, in part, will be apparent from the description, or may be learned by practice of the presented embodiments.

According to one or more embodiments of the present disclosure, a light receiving element includes a first electrode, a second electrode facing the first electrode, and a light receiving layer between the first electrode and the second electrode, wherein the light receiving layer includes a first compound represented by Formula 1.

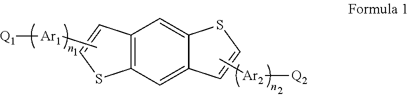

In Formula 1, n₁ and n₂ may each independently be an integer of 0 to 10. Ar₁ and Ar₂ may each independently be a substituted or unsubstituted arylene group having 6 to 60 carbon atoms or a substituted or unsubstituted heteroarylene group having 2 to 60 carbon atoms. Q₁ and Q₂ may each independently be represented by any one selected from among Formulas Q-1 to Q-7.

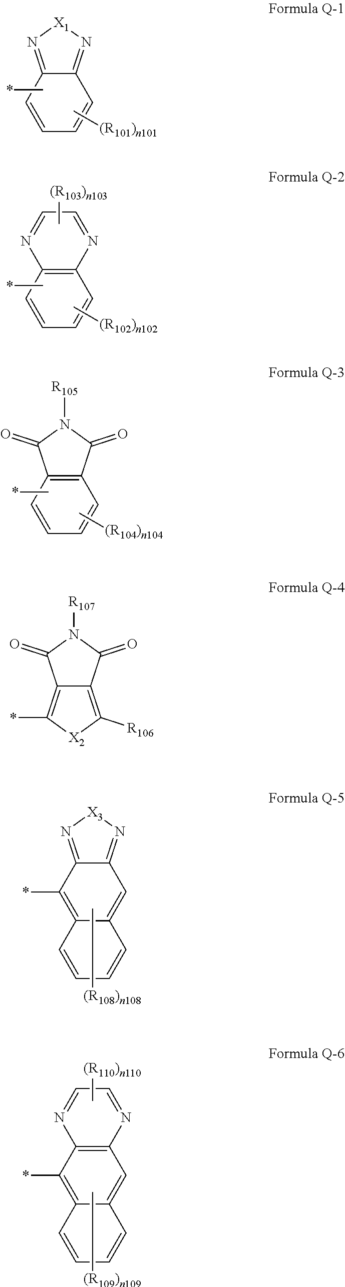

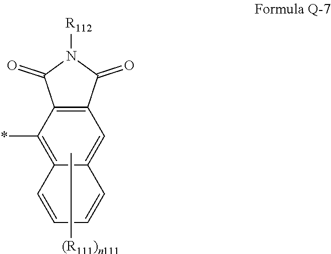

In Formulas Q-1 to Q-7, n₁₀₁, n₁₀₂, and n₁₀₄ may each independently be an integer of 0 to 3. n₁₀₃ and n₁₁₀ may each independently be an integer of 0 to 2. n₁₀₈, n₁₀₉, and n₁₁₁ may each independently be an integer of 0 to 5. X₁ to X₃ may each independently be oxygen, sulfur, selenium, or tellurium.

R₁₀₁ to R₁₀₃ may each independently be hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms. R₁₀₄ to R₁₁₂ may each independently be hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms. [symbol] is a site linked to Formula 1.

In one or more embodiments, in Formula 1, if (e.g., when) Q₁ and Q₂ are each represented by Formula Q-1, Q₁ and Q₂ may each independently be represented by any one selected from among Formulas R-11 to R-19.

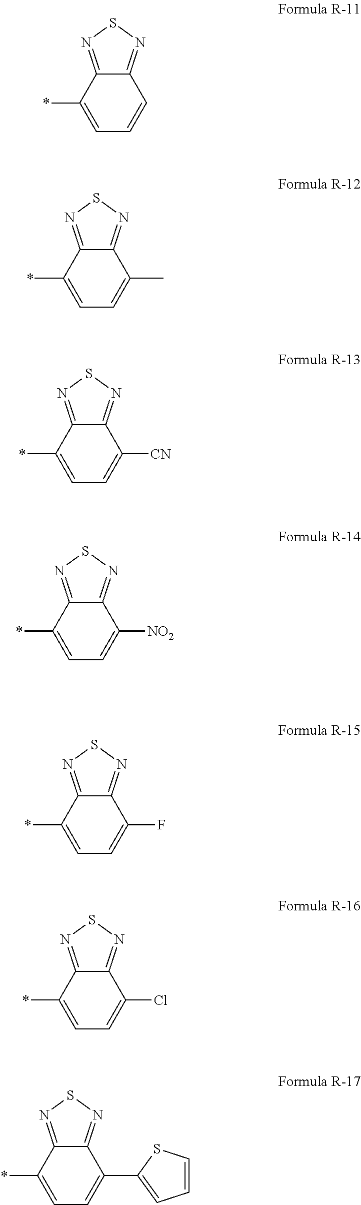

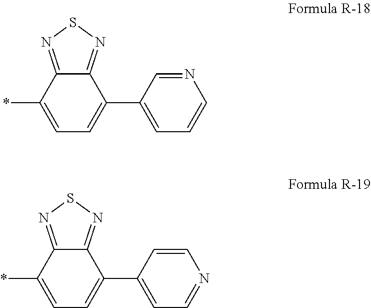 [SMILES: *c1ccc(-c2cccnc2)c2nsnc12 ; *c1ccc(-c2ccncc2)c2nsnc12]

In Formulas R-11 to R-19, [symbol] is a site linked to Formula 1.

In one or more embodiments, in Formula 1, if (e.g., when) Q₁ and Q₂ are each represented by Formula Q-2, Q₁ and Q₂ may each independently be represented by any one selected from among Formulas R-21 to R-24.

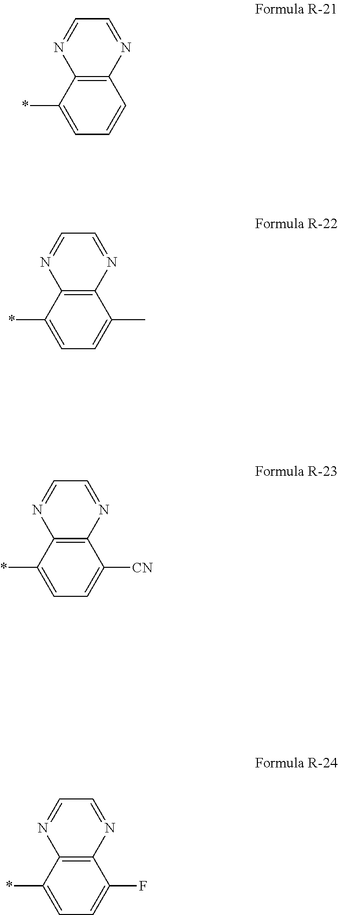 [SMILES: *c1cccc2nccnc12 ; *c1ccc(C)c2nccnc12 ; *c1ccc(C#N)c2nccnc12 ; *c1ccc(F)c2nccnc12]

In Formulas R-21 to R-24, [symbol] is a site linked to Formula 1.

In one or more embodiments, in Formula 1, if (e.g., when) Q₁ and Q₂ are each represented by Formula Q-3, Q₁ and Q₂ may each independently be represented by any one selected from among Formulas R-31 to R-34.

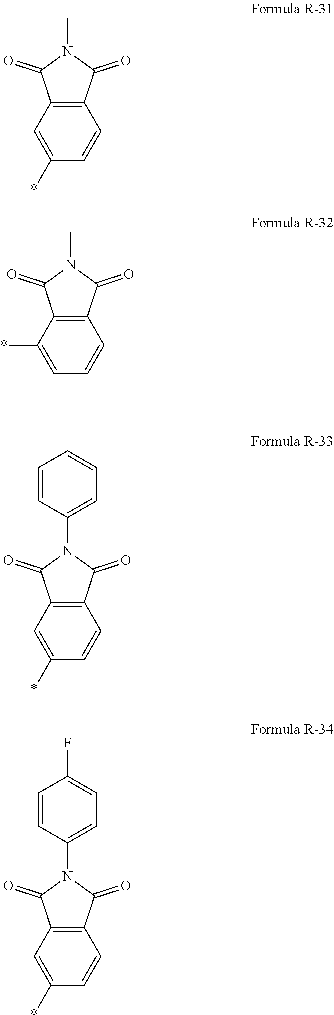 [SMILES: *c1ccc2c(c1)C(=O)N(C)C2=O ; *c1cccc2c1C(=O)N(C)C2=O ; *c1ccc2c(c1)C(=O)N(c1ccccc1)C2=O ; *c1ccc2c(c1)C(=O)N(c1ccc(F)cc1)C2=O]

In Formulas R-31 to R-34, [symbol] is a site linked to Formula 1.

In one or more embodiments, in Formula 1, if (e.g., when) Q₁ and Q₂ are each represented by Formula Q-4, Q₁ and Q₂ may each independently be represented by Formula R-41 or Formula R-42.

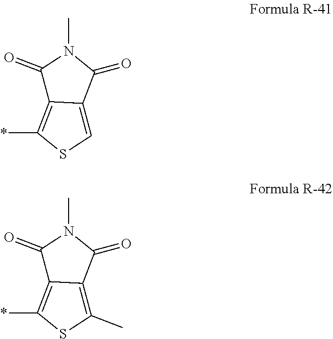

In Formulas R-41 and R-42, -* is a site linked to Formula 1.

In one or more embodiments, in Formula 1, if (e.g., when) Q₁ and Q₂ are each represented by Formula Q-5, Q₁ and Q₂ may each independently be represented by Formula R-51 or Formula R-52.

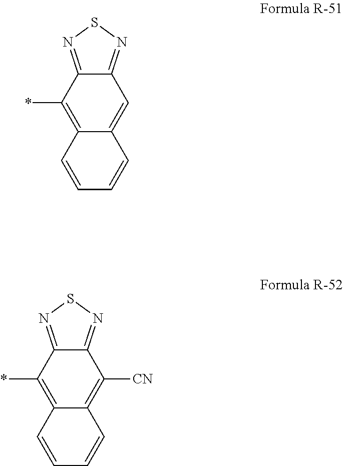 [SMILES: *c1c2ccccc2cc2nsnc12 ; *c1c2ccccc2c(C#N)c2nsnc12]

In Formulas R-51 and R-52, -* is a site linked to Formula 1.

In one or more embodiments, in Formula 1, if (e.g., when) Q₁ and Q₂ are each represented by Formula Q-6, Q₁ and Q₂ may each independently be represented by Formula R-61.

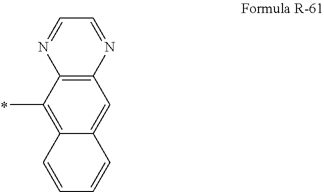

In Formula R-61, -* is a site linked to Formula 1.

In one or more embodiments, in Formula 1, if (e.g., when) Q₁ and Q₂ are each represented by Formula Q-7, Q₁ and Q₂ may each independently be represented by Formula R-71.

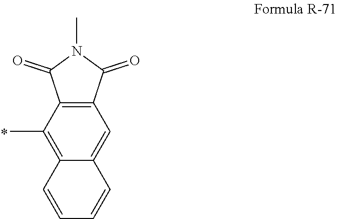

In Formula R-71, -* is a site linked to Formula 1.

In one or more embodiments, the first compound represented by Formula 1 may be represented by any one selected from among Formulas A-1 to A-5.

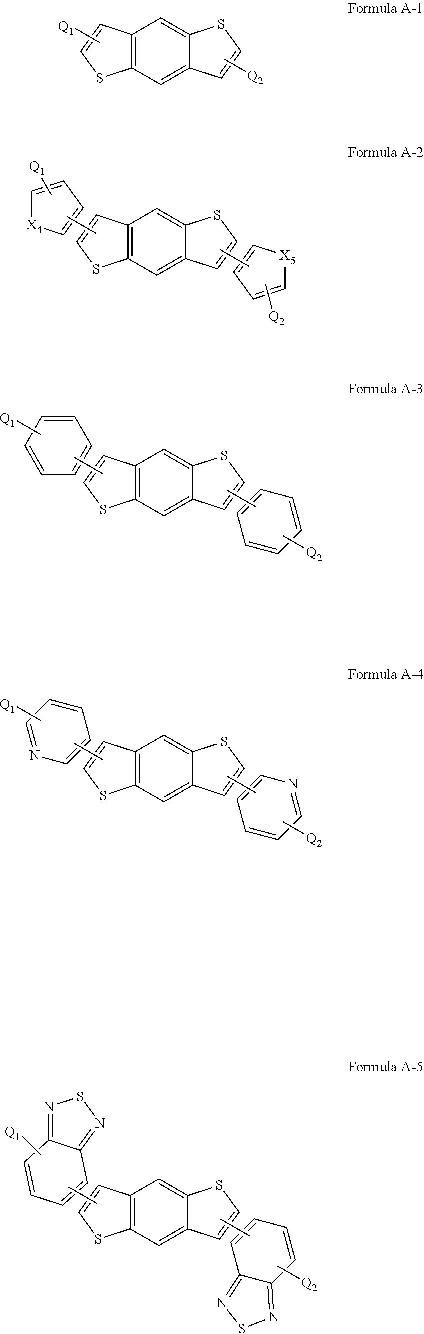

In Formulas A-1 to A-5, X₄ and X₅ may each independently be oxygen, sulfur, selenium, or tellurium. Q₁ and Q₂ may each independently be the same as defined in Formula 1.

In one or more embodiments, the first compound represented by Formula 1 may be represented by any one selected from among Formulas 1-1 to 1-6.

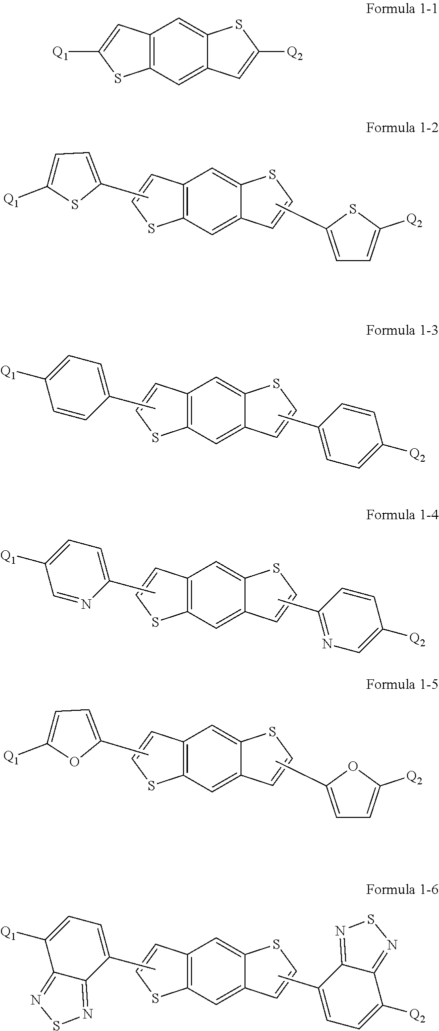

In Formulas 1-1 to 1-6, Q₁ and Q₂ may each independently be the same as defined in Formula 1.

In one or more embodiments, the light receiving layer may further include a second compound represented by Formula 2.

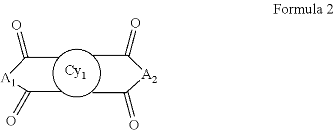

In Formula 2, Cy₁ may be a substituted or unsubstituted aromatic hydrocarbon ring having 6 to 30 carbon atoms. A₁ and A₂ may each independently be oxygen or NR₁₁₃. R₁₁₃ may be hydrogen, deuterium, a halogen, a substituted or unsubstituted alkyl group having 1 to 30 carbon atoms, a substituted or unsubstituted aryl group having 6 to 30 carbon atoms, a substituted or unsubstituted heteroaryl group having 2 to 30 carbon atoms, or a substituted or unsubstituted cycloalkyl group having 3 to 30 carbon atoms.

In one or more embodiments, the light receiving layer may include a light absorption layer containing the first compound and a photoelectric conversion layer containing the second compound.

In one or more embodiments, the photoelectric conversion layer may be on the light absorption layer.

In one or more embodiments, an interface may be defined between the light absorption layer and the photoelectric conversion layer.

In one or more embodiments, the light emitting element may further include a hole control layer between the first electrode and the light receiving layer, and an electron control layer between the light receiving layer and the second electrode.

In one or more embodiments, the first compound represented by Formula 1 may be any one selected from among compounds of Compound Group 1, and the second compound represented by Formula 2 may be any one selected from among compounds of Compound Group 2.

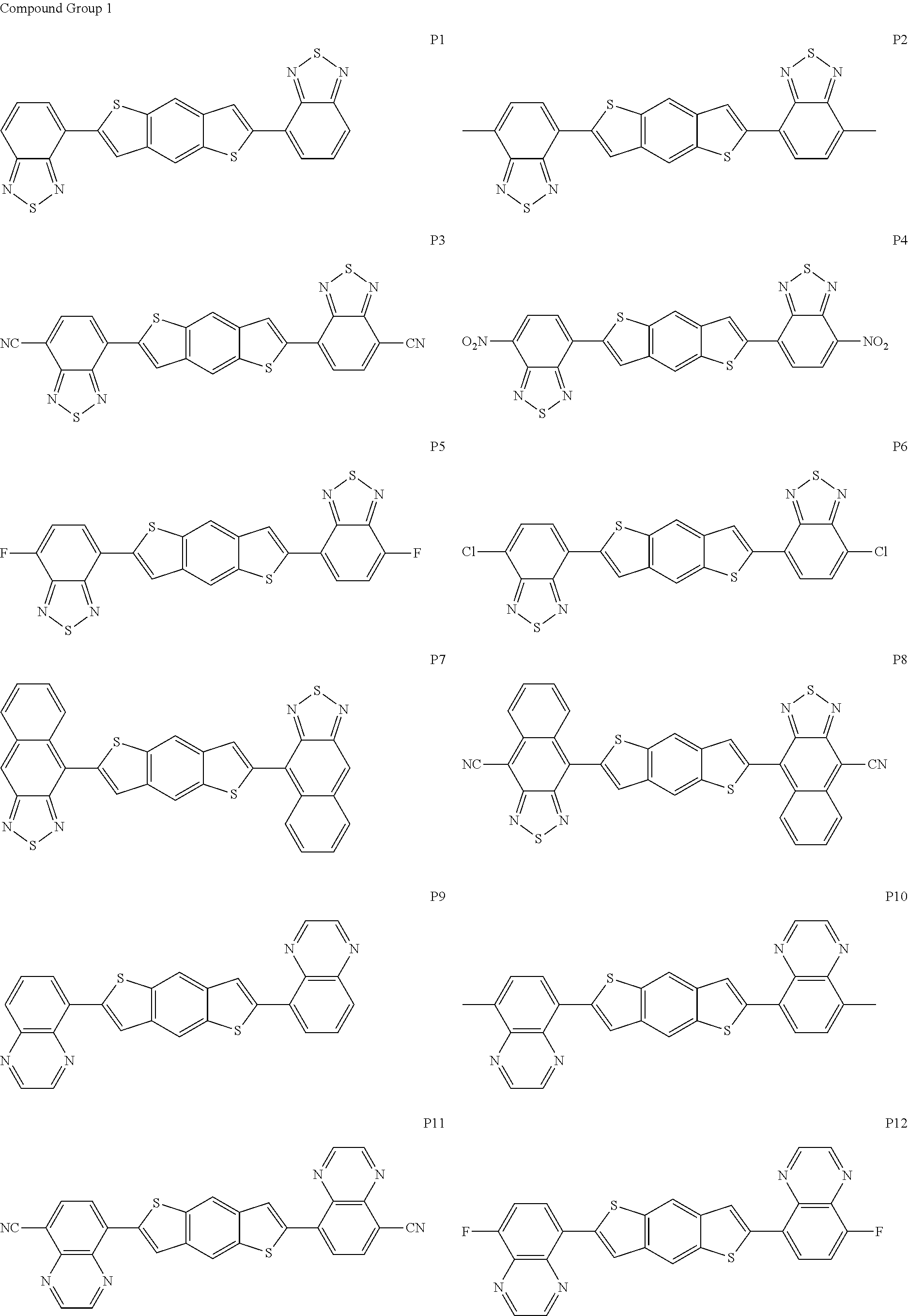

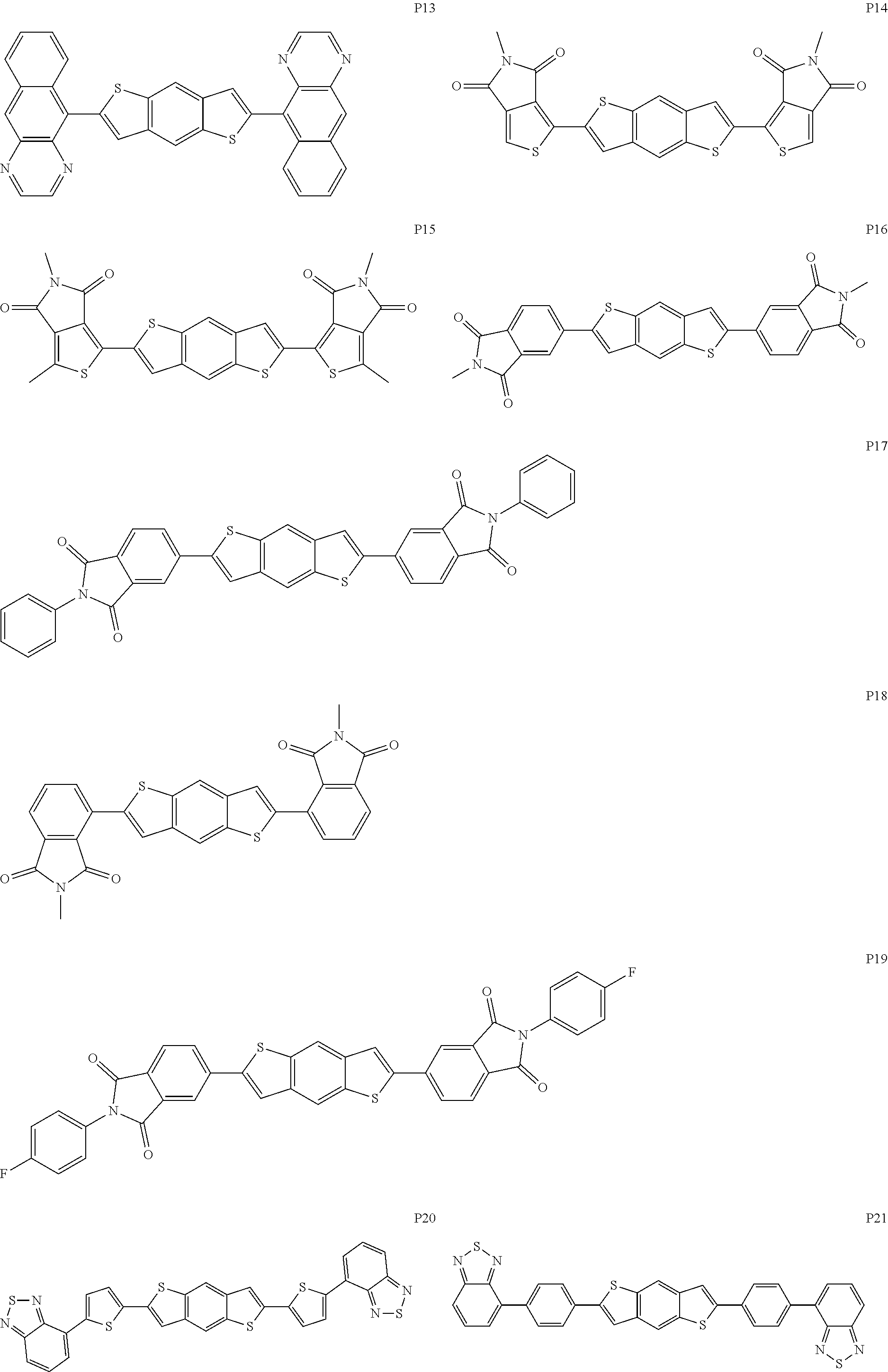

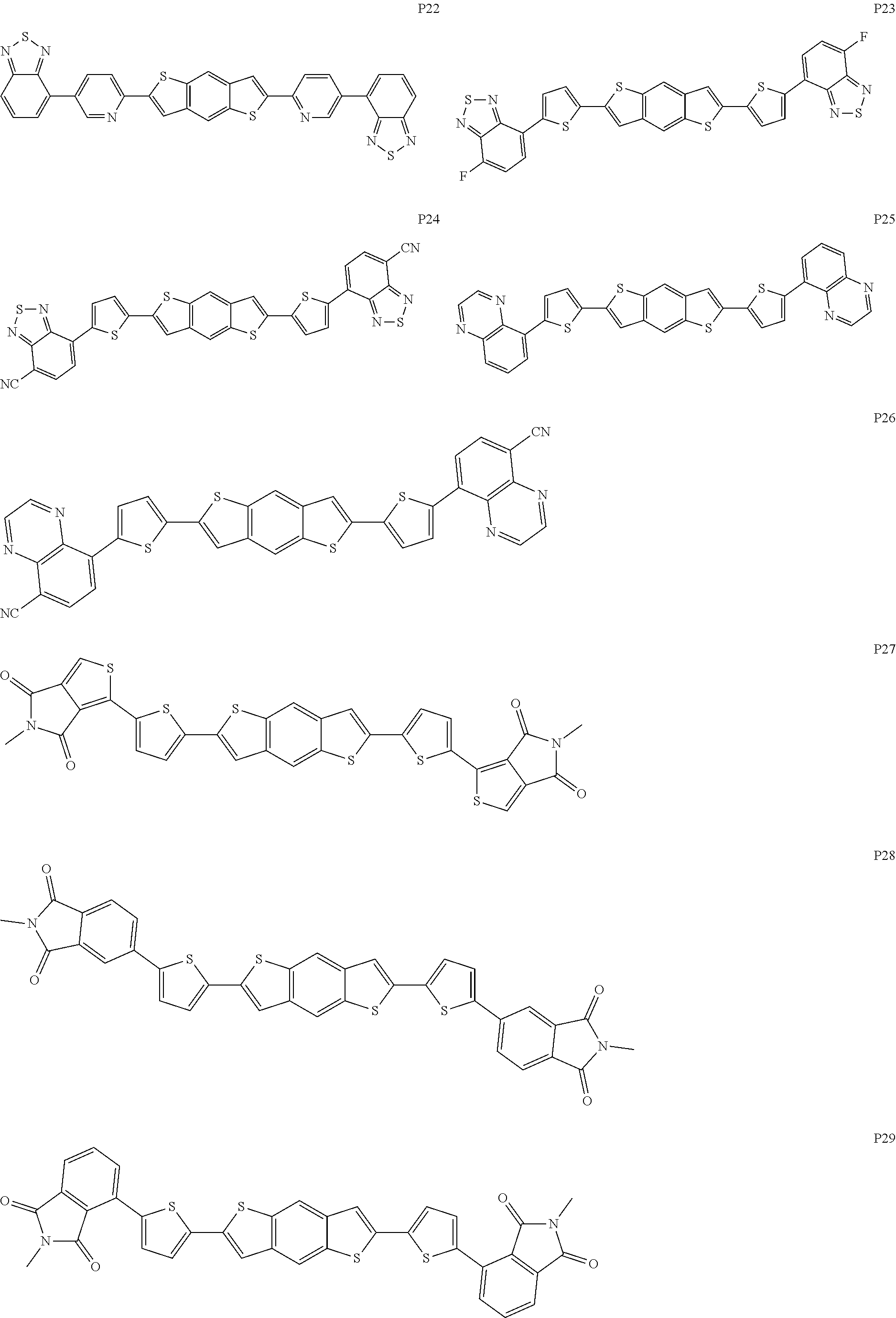

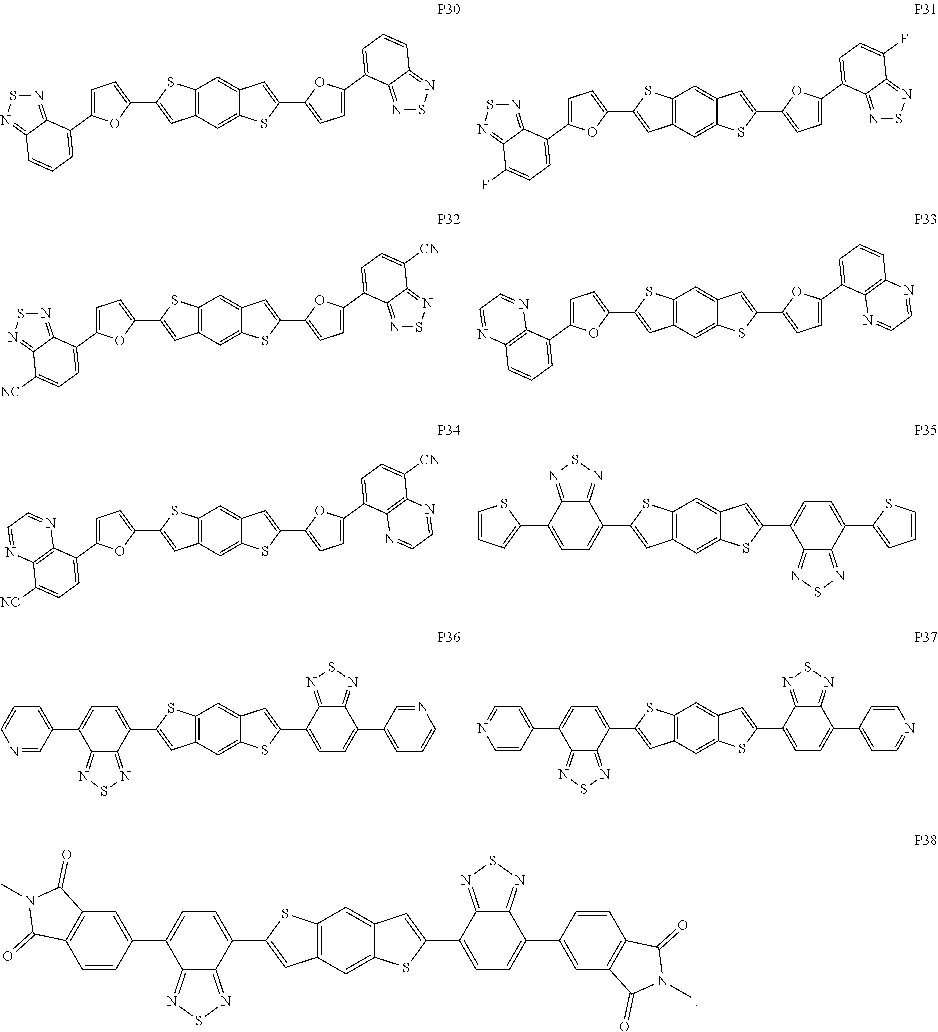

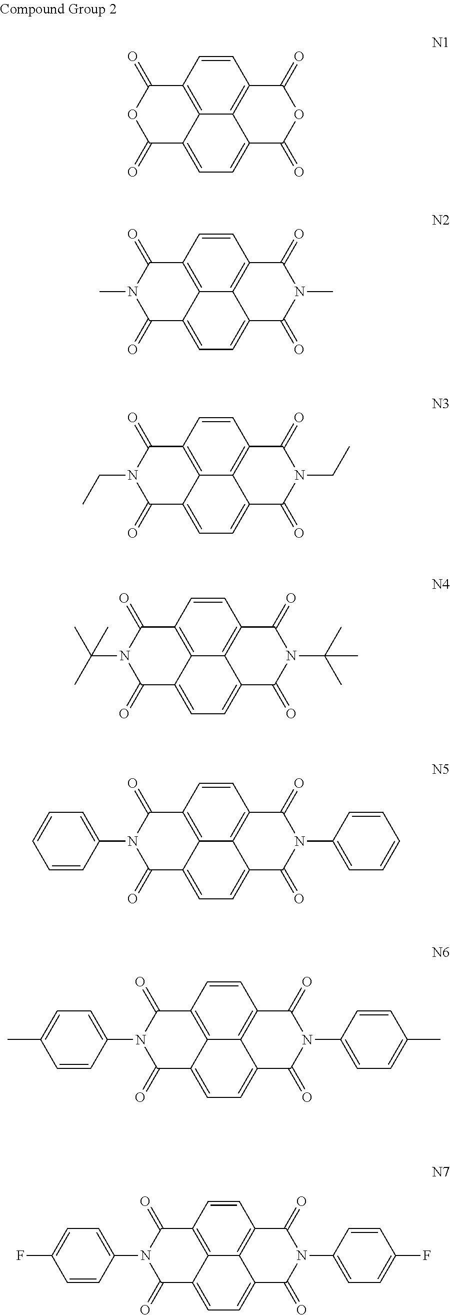

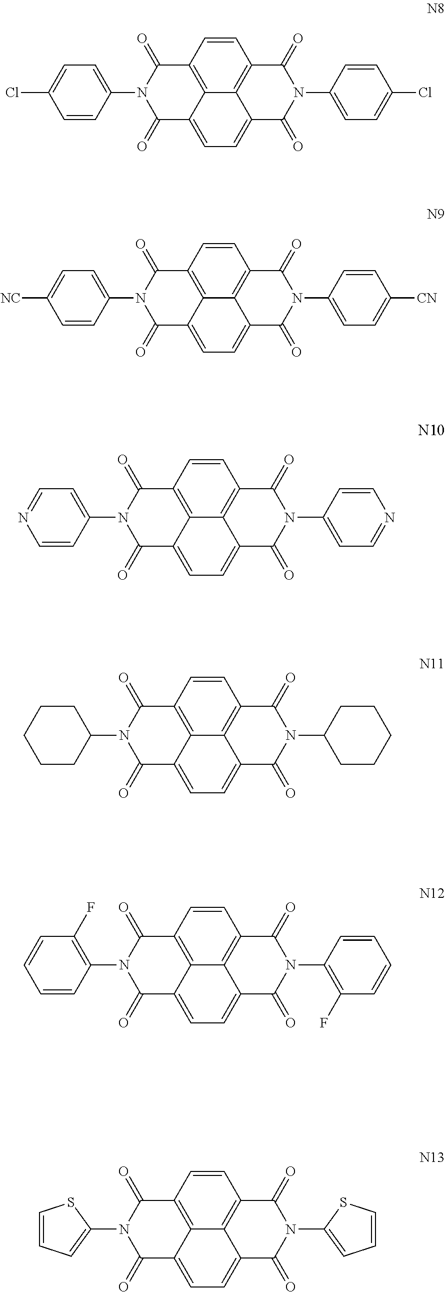 [SMILES: O=C1c2ccc3c4c(ccc(c24)C(=O)N1c1ccc(Cl)cc1)C(=O)N(c1ccc(Cl)cc1)C3=O ; N#Cc1ccc(N2C(=O)c3ccc4c5c(ccc(c35)C2=O)C(=O)N(c2ccc(C#N)cc2)C4=O)cc1 ; O=C1c2ccc3c4c(ccc(c24)C(=O)N1c1ccncc1)C(=O)N(c1ccncc1)C3=O ; O=C1c2ccc3c4c(ccc(c24)C(=O)N1C1CCCCC1)C(=O)N(C1CCCCC1)C3=O ; O=C1c2ccc3c4c(ccc(c24)C(=O)N1c1ccccc1F)C(=O)N(c1ccccc1F)C3=O ; O=C1c2ccc3c4c(ccc(c24)C(=O)N1c1cccs1)C(=O)N(c1cccs1)C3=O]

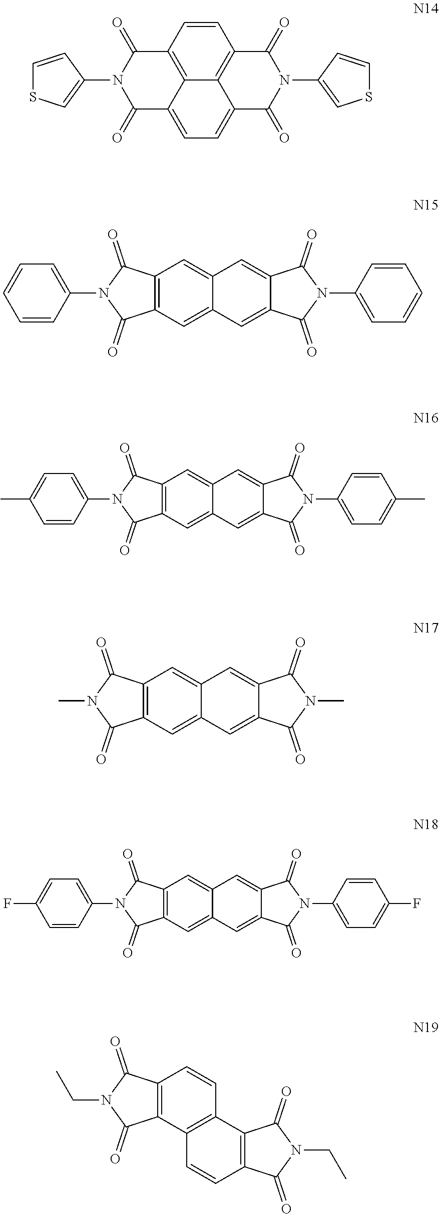

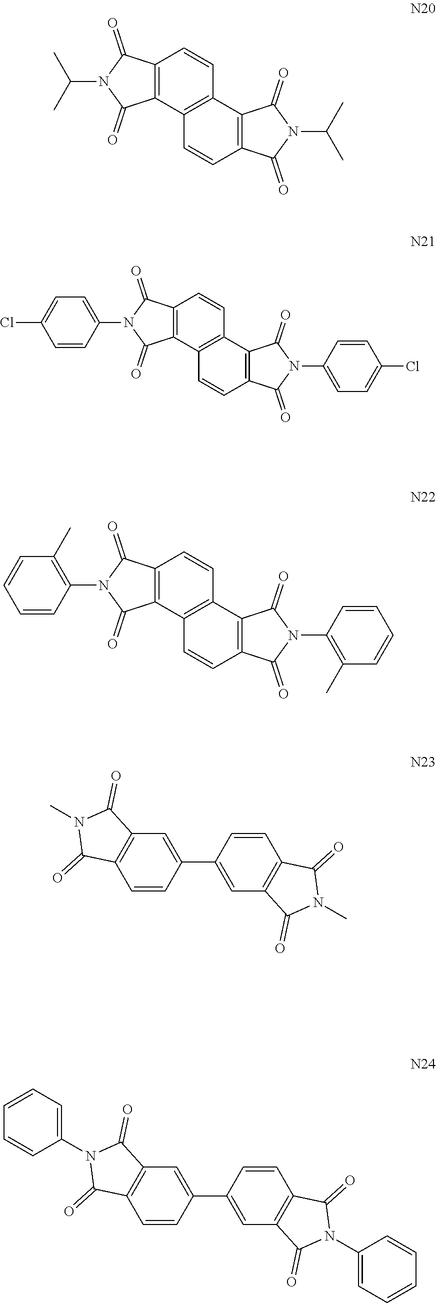

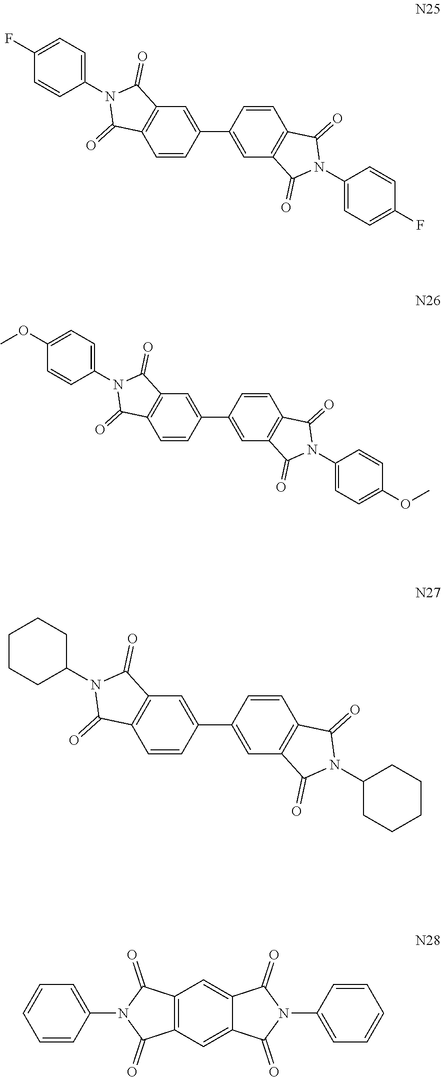

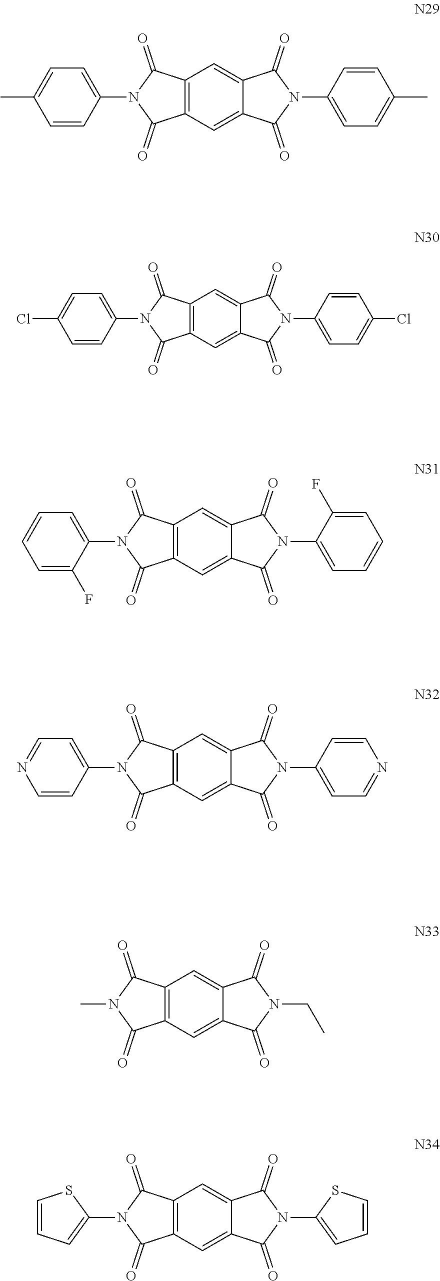

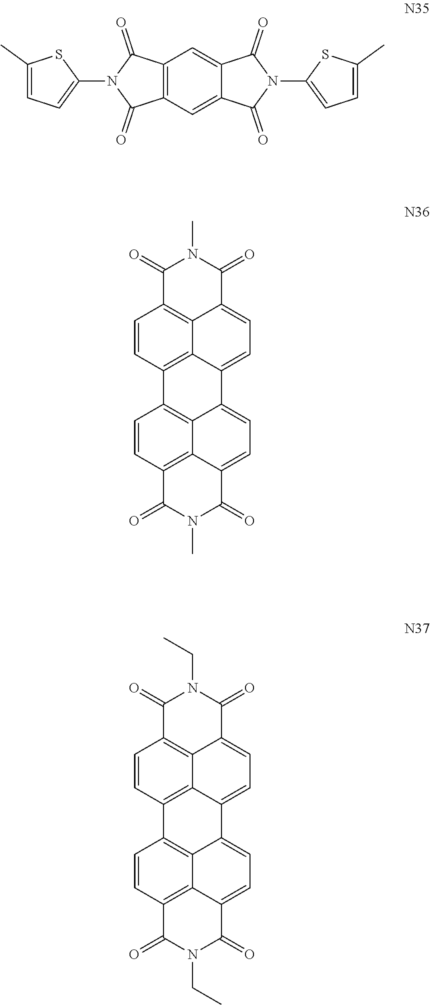

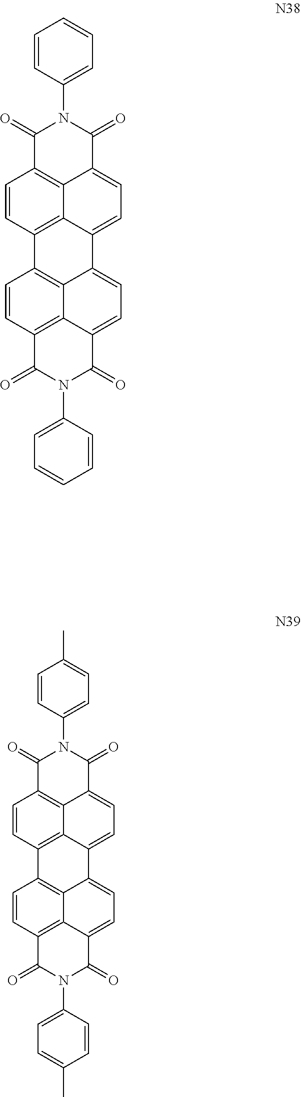 [SMILES: O=C1c2ccc3c4ccc5c6c(ccc(c7ccc(c2c37)C(=O)N1c1ccccc1)c64)C(=O)N(c1ccccc1)C5=O ; Cc1ccc(N2C(=O)c3ccc4c5ccc6c7c(ccc(c8ccc(c3c48)C2=O)c75)C(=O)N(c2ccc(C)cc2)C6=O)cc1]

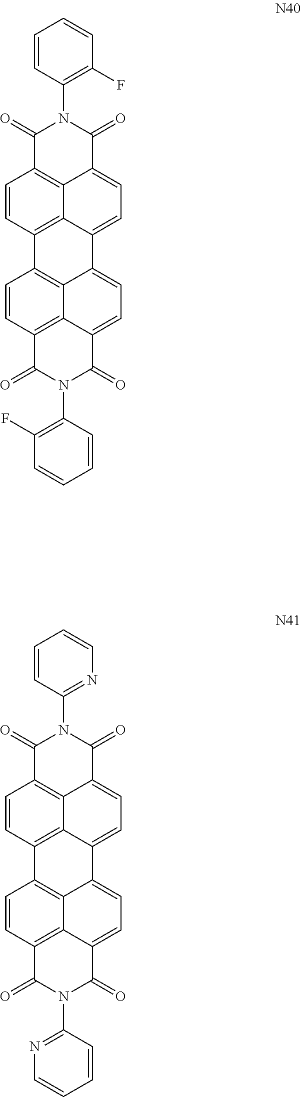 [SMILES: O=C1c2ccc3c4ccc5c6c(ccc(c7ccc(c2c37)C(=O)N1c1ccccc1F)c64)C(=O)N(c1ccccc1F)C5=O ; O=C1c2ccc3c4ccc5c6c(ccc(c7ccc(c2c37)C(=O)N1c1ccccn1)c64)C(=O)N(c1ccccn1)C5=O]

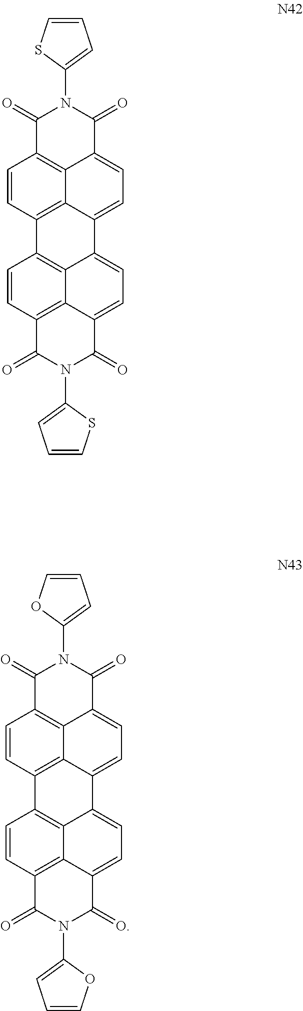 [SMILES: O=C1c2ccc3c4ccc5c6c(ccc(c7ccc(c2c37)C(=O)N1c1cccs1)c64)C(=O)N(c1cccs1)C5=O ; O=C1c2ccc3c4ccc5c6c(ccc(c7ccc(c2c37)C(=O)N1c1ccco1)c64)C(=O)N(c1ccco1)C5=O]

In one or more embodiments of the present disclosure, an electronic device includes a base layer and a display element layer including a light emitting element and a light receiving element, wherein the light emitting element includes a first light emitting electrode on the base layer, an emission layer on the first light emitting electrode, and a second light emitting electrode on the emission layer, the light receiving element includes a first light receiving electrode on the base layer, a light receiving layer on the first light receiving electrode, and a second light receiving electrode on the light receiving layer, and the light receiving layer includes a first compound represented by Formula 1.

In one or more embodiments of the present disclosure, a polycyclic compound is represented by Formula 1.

### BRIEF DESCRIPTION OF DRAWINGS

The accompanying drawings are included to provide a further understanding of the present disclosure, and are incorporated in and constitute a part of this disclosure. The drawings illustrate embodiments of the present disclosure and, together with the description, serve to explain principles of the present disclosure. Above and/or other aspects of the present disclosure should become apparent and appreciated from the following description of embodiments taken in conjunction with the accompanying drawings. In the drawings:

FIG. 1 is a perspective view of an electronic device of one or more embodiments of the present disclosure;

FIG. 2 is an exploded perspective view of an electronic device of one or more embodiments of the present disclosure;

FIG. 3 is a cross-sectional view of an electronic device of one or more embodiments of the present disclosure;

FIG. 4 is a plan view of a portion of a display module according to one or more embodiments of the present disclosure;

FIG. 5 is a cross-sectional view showing a portion of a display module according to one or more embodiments of the present disclosure;

FIG. 6 is a cross-sectional view showing an electronic device according to one or more embodiments of the present disclosure;

FIG. 7A is a cross-sectional view showing a light emitting element according to one or more embodiments of the present disclosure;

FIG. 7B is a cross-sectional view showing a light receiving element according to one or more embodiments of the present disclosure;

FIG. 8A is a cross-sectional view showing a light emitting element according to one or more embodiments of the present disclosure;

FIG. 8B is a cross-sectional view showing a light receiving element according to one or more embodiments of the present disclosure; and

FIG. 9 is a cross-sectional view showing a portion of a display module according to one or more embodiments of the present disclosure.

### DETAILED DESCRIPTION

The present disclosure may be modified in many alternate forms, and thus one or more embodiments will be exemplified in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

In describing the drawings, like reference numerals are utilized for like elements. In the drawings, the sizes of elements may be exaggerated for clarity. It will be understood that, although the terms “first,” “second,” and/or the like may be utilized herein to describe one or more suitable elements, these elements should not be limited by these terms. These terms are only utilized to distinguish one element from another element. For example, a first element may be referred to as a second element, and similarly, a second element may be referred to as a first element without departing from the teachings of the present disclosure. As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure.”

In the present disclosure, it should be understood that the terms, “comprise(s)/include(s)/have (has)” and “comprising/including/having” are intended to specify the presence of stated features, integers, steps, operations, elements, components, and/or a (e.g., any suitable) combination thereof in the disclosure, but do not preclude the presence or addition of one or more other features, integers, steps, operations, elements, components, and/or a (e.g., any suitable) combination thereof.

In the present disclosure, it should be understood that if (e.g., when) an element such as a layer, a film, a region, or a substrate is referred to as being “on” or “above” another element, it may be “directly on” the other element or intervening elements may also be present therebetween. However, “directly on” may refer to that there are no additional layers, films, regions, substrates, etc., between a layer, a film, a region, or a substrate and the other element. In contrast, it should be understood that if (e.g., when) an element such as a layer, a film, a region, or a substrate is referred to as being “beneath” or “under” another element, it may be “directly under” the other element or intervening elements may also be present. In addition, in the present disclosure, it should be understood that if (e.g., when) an element is referred to as being “on”, it may be as being “above” or “under” the other element.

In the present disclosure, the term “substituted or unsubstituted” may indicate that one is substituted or unsubstituted with at least one substituent selected from among the group consisting of deuterium, a halogen, a cyano group, a nitro group, an amino group, a silyl group, a germyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In one or more embodiments, each of the substituents presented may be substituted or unsubstituted. For example, a biphenyl group may be interpreted as an aryl group or as a phenyl group substituted with a phenyl group.

As utilized herein, the term “bonded to an adjacent group to form (or provide) a ring” may indicate that one group is bonded to an adjacent group to form (or provide) a substituted or unsubstituted hydrocarbon ring or a substituted or unsubstituted heterocycle. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may each be monocyclic or polycyclic. In one or more embodiments, the rings formed by adjacent groups being bonded to each other may be linked to another ring to form (or provide) a spiro structure.

As utilized herein, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as mutually “adjacent groups”, and two ethyl groups in 1,1-diethylcyclopentane may be interpreted as mutually “adjacent groups”. In one or more embodiments, two methyl groups in 4,5-dimethylphenanthrene may be interpreted as mutually “adjacent groups”.

As utilized herein, examples of a halogen may include fluorine, chlorine, bromine, and iodine.

As utilized herein, an alkyl group may be linear or branched. The number of carbon atoms in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but embodiments of present disclosure are not limited thereto.

As utilized herein, a cycloalkyl group may refer to a cyclic alkyl group. The number of carbon atoms in the cycloalkyl group may be 3 to 50, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but embodiments of the present disclosure are not limited thereto.

As utilized herein, an alkenyl group refers to a hydrocarbon group including at least one carbon-carbon double bond in the middle or end of an alkyl group having 2 or more carbon atoms. The alkenyl group may be linear or branched. The number of carbon atoms is not particularly limited, for example, may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, and/or the like, but embodiments of the present disclosure are not limited thereto.

As utilized herein, an alkynyl group refers to a hydrocarbon group including at least one carbon-carbon triple bond in the middle or end of an alkyl group having 2 or more carbon atoms. The alkynyl group may be linear or branched. The number of carbon atoms is not particularly limited, for example, may be 2 to 30, 2 to 20, or 2 to 10. Examples of the alkynyl group may include an ethynyl group, a propynyl group, and/or the like, but embodiments of the present disclosure are not limited thereto.

As utilized herein, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

As utilized herein, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but embodiments of the present disclosure are not limited thereto.

As utilized herein, a fluorenyl group may be substituted, and two substituents may be bonded to each other to form (or provide) a spiro structure. Examples that the fluorenyl group is substituted are as follows. However, embodiments of the present disclosure are not limited thereto.

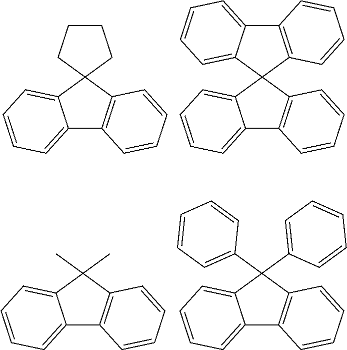

As utilized herein, a heterocyclic group refers to any functional group or substituent derived from a ring containing at least one of B, O, N, P, Si, S, Se, or Te as a hetero atom. The heterocyclic group may include an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

As utilized herein, a heterocyclic group may contain at least one of B, O, N, P, Si, S, Se, or Te as a hetero atom. When the heterocyclic group contains two or more hetero atoms, the two or more hetero atoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 30, 2 to 20, or 2 to 10.

As utilized herein, an aliphatic heterocyclic group may contain at least one of B, O, N, P, Si, S, Se, or Te as a hetero atom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but are not limited to thereto

As utilized herein, a heteroaryl group may contain at least one of B, O, N, P, Si, S, Se, or Te as a hetero atom. When the heteroaryl group contains two or more hetero atoms, the two or more hetero atoms may be the same as or different from each other. The heteroaryl group may be a monocyclic heteroaryl group or a polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, an indolocarbazole group, and/or the like, but embodiments of the present disclosure are not limited thereto.

As utilized herein, the above description of the aryl group may be applied to an arylene group, except that the arylene group is a divalent group. The above description of the heteroaryl group may be applied to a heteroarylene group, except that the heteroarylene group is a divalent group.

As utilized herein, the number of carbon atoms in a carbonyl group is not particularly limited, for example, may be 1 to 40, 1 to 30, or 1 to 20. For example, the carbonyl group may have the following structure, but embodiments of the present disclosure are not limited thereto.

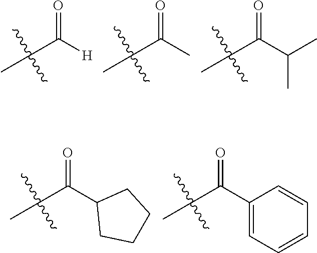

As utilized herein, the number of carbon atoms in a sulfinyl group and/or a sulfonyl group is not particularly limited, for example, may be 1 to 30. The sulfinyl group may include an alkyl sulfinyl group and/or an aryl sulfinyl group. The sulfonyl group may include an alkyl sulfonyl group and/or an aryl sulfonyl group.

As utilized herein, a thio group may include an alkyl thio group and/or an aryl thio group. The thio group may indicate a group that a sulfur atom is bonded to an alkyl group or an aryl group defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, and/or the like, but embodiments of the present disclosure are not limited to thereto.

As utilized herein, an oxy group may indicate a group that an oxygen atom is bonded to an alkyl group or aryl group defined above. The oxy group may include an alkoxy group and/or an aryl oxy group. The alkoxy group may be linear, branched, or cyclic. The number of carbon atoms in the alkoxy group is not particularly limited, but may be, for example, 1 to 20, or 1 to 10. Examples of the oxy group include methoxy, ethoxy, n-propoxy, isopropoxy, butoxy, pentyloxy, hexyloxy, octyloxy, nonyloxy, decyloxy, benzyloxy, and/or the like, but embodiments of the present disclosure are not limited thereto.

As utilized herein, a boron group may refer to a group that a boron atom is bonded to an alkyl group or aryl group defined above. The boron group may include an alkyl boron group and/or an aryl boron group. Examples of the boron group may include a dimethyl boron group, a diethyl boron group, a t-butylmethyl boron group, a diphenyl boron group, a phenyl boron group, and/or the like, but embodiments of the present disclosure are not limited thereto.

In the present disclosure, an alkenyl group may be linear or branched. The number of carbon atoms in the alkenyl group is not particularly limited, for example, may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, and/or the like, but embodiments of the present disclosure are not limited thereto.

As utilized herein, the number of carbon atoms in an amine group is not particularly limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, a triphenylamine group, and/or the like, but embodiments of the present disclosure are not limited thereto.

As utilized herein, the above-described examples of the alkyl group may also apply to an alkylthio group, an alkyl sulfoxy group, an alkyl aryl group, an alkylamino group, an alkyl boron group, an alkyl silyl group, and an alkyl amine group.

As utilized herein, the above-described examples of the aryl group may also apply to an aryloxy group, an arylthio group, an aryl sulfoxy group, an aryl amino group, an aryl boron group, an aryl silyl group, and an aryl amine group.

As utilized herein, a direct linkage may refer to a single bond.

In the present disclosure, “ [symbol] ” and “-*” each refer to a position to be connected.

Hereinafter, embodiments of the present disclosure will be described with reference to the accompanying drawings.

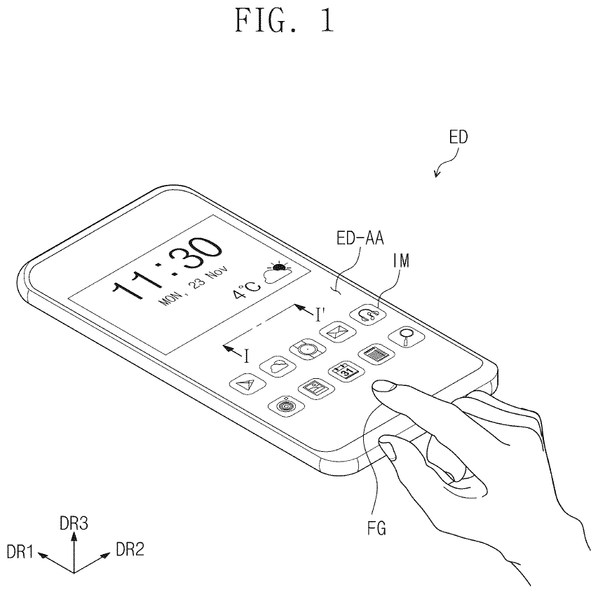

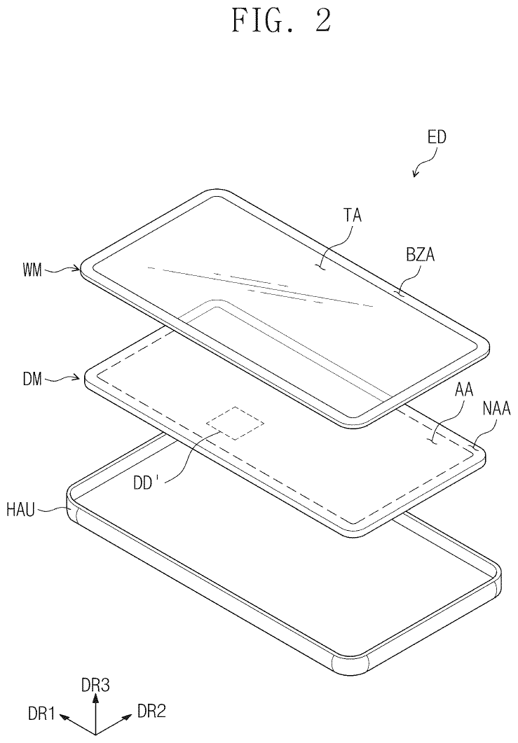

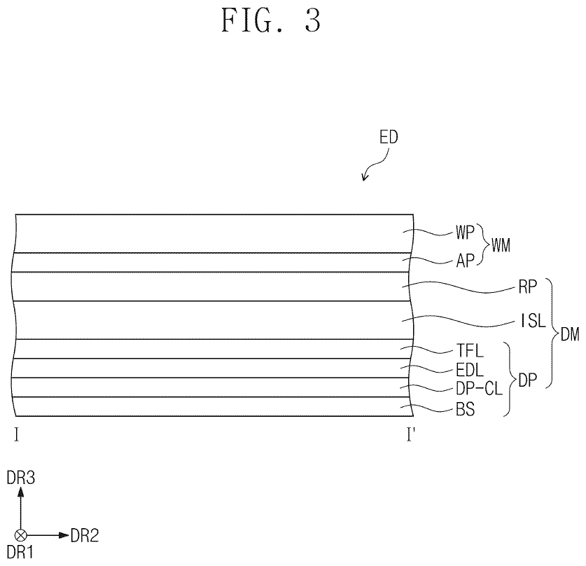

FIG. 1 is a perspective view showing an electronic device according to one or more embodiments of the present disclosure. FIG. 2 is an exploded perspective view showing an electronic device according to one or more embodiments of the present disclosure. FIG. 3 is a cross-sectional view schematically showing a portion corresponding to line I-I′ of FIG. 1.

An electronic device ED of one or more embodiments shown in FIGS. 1-3 may be a device activated according to electrical signals. For example, the electronic device ED may be a mobile phone, a tablet, a car navigation system, a game console, or a wearable device, but embodiments of the present disclosure are not limited thereto. In FIG. 1, a mobile phone is shown as the electronic device ED by way of example.

The electronic device ED may display an image IM through an active region ED-AA. The active region ED-AA may include a plane defined by a first direction axis DR1 and a second direction axis DR2. The active region ED-AA may further include a curved surface bent from at least one side of the plane defined by the first direction axis DR1 and the second direction axis DR2. For example, in one or more embodiments, the active region ED-AA may include the plane alone, and the active region ED-AA may further include curved surfaces each bent from at least two sides of the plane (e.g., four curved surfaces each bent from four sides of the plane).

In one or more embodiments, FIG. 1 and the following drawings show the first to fourth direction axes DR1 to DR4, and directions indicated by the first to fourth direction axes DR1 DR2, DR3, and DR4 as described herein are relative concepts, and may thus be changed to other directions. In one or more embodiments, the directions indicated by the first to fourth direction axes DR1 DR2, DR3, and DR4 may be described as first to fourth directions, and the same reference numerals may be utilized.

The first direction axis DR1 and the second direction axis DR2 herein may be normal (e.g., perpendicular) to each other, and the third direction axis DR3 may be a normal direction with respect to a plane defined by the first direction axis DR1 and the second direction axis DR2. The fourth direction axis DR4 may be a direction between the first direction axis DR1 and the second direction axis DR2.

A thickness direction of the electronic device ED may be parallel to the third direction axis DR3 which is a normal direction with respect to a plane defined by the first direction axis DR1 and the second direction axis DR2. As described herein, a front surface (or an upper surface) and a rear surface (or a lower surface) of members constituting the electronic device ED may be defined with respect to the third direction axis DR3.

The image IM provided in electronic device ED of one or more embodiments may include still images as well as dynamic images. In FIG. 1, a watch window and icons are shown as an example of the image IM. A surface on which the image IM is displayed may correspond to a front surface of the electronic device ED and may also correspond to a front surface of a window member WM.

In one or more embodiments, the electronic device ED may detect user inputs applied from the outside. For example, the user inputs may include one or more suitable types (kinds) of external inputs such as a portion of the user's body, light, heat, and/or pressure. The electronic device ED of one or more embodiments may detect the user inputs through the active region ED-AA and respond to detected input signals. In one or more embodiments, the user inputs applied to a side surface and/or a rear surface of the electronic device ED may also be detected depending on a provided design of the electronic device ED, and embodiments of the present disclosure are not limited to.

For example, the electronic device ED according to one or more embodiments may detect biometric information such as a user's fingerprint FG applied from the outside. A fingerprint recognition region may be provided in the active region ED-AA of the electronic device ED. The fingerprint recognition region may be provided in an entire region of the active region ED-AA or may be provided in some region of the active region ED-AA. In one or more embodiments, the fingerprint recognition region may be an external input recognition region that detects not only the user's biometric information but also inputs provided from the outside.

Referring to FIGS. 2 and 3, the electronic device ED of one or more embodiments may include a display module DM, a window member WM, and a housing HAU. In one or more embodiments, the window member WM and the housing HAU may be bonded together to form (or provide) an exterior of the electronic device ED.

The display module DM according to one or more embodiments may be divided into an active region AA and a peripheral region NAA. The active region AA may be a region activated according to electrical signals. As described above, the active region AA may be a portion that displays images and/or detects external inputs.

The peripheral region NAA may be a region adjacent to at least one side of the active region AA. The peripheral region NAA may be arranged to be around (e.g., surround) the active region AA. However, embodiments of the present disclosure are not limited thereto, for example, a portion of the peripheral region NAA may not be provided in one or more embodiments. A driving circuit, a driving line, and/or the like for driving the active region AA may be arranged in the peripheral region NAA.

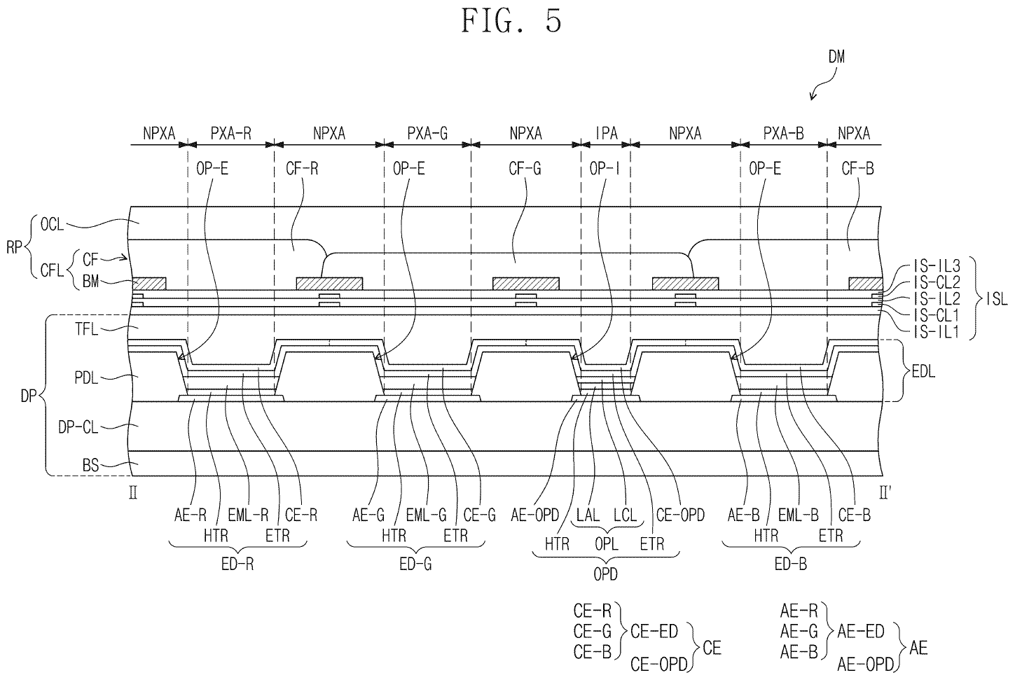

The electronic device ED according to one or more embodiments may include light emitting elements ED-R, ED-G, and ED-B (FIG. 5) and a light receiving element OPD (see FIG. 5). For example, the electronic device ED according to one or more embodiments may display images through the operation of the light emitting elements ED-R, ED-G, and ED-B, and the light receiving element OPD (see FIG. 5) and detect external inputs through the operation of the light receiving element OPD (see FIG. 5).

The display module DM according to one or more embodiments may include a display panel DP and an input sensing layer ISL arranged on the display panel DP. In one or more embodiments, the display module DM may include an anti-reflection member RP. In one or more embodiments, the anti-reflection member RP may be arranged on the input sensing layer ISL. In one or more embodiments, if (e.g., when) the input sensing layer ISL is not provided, the anti-reflection member RP may be arranged on the display panel DP.

The display panel DP may include a base layer BS and a display element layer EDL arranged on the base layer BS. In one or more embodiments, the display panel DP may include a base layer BS, a circuit layer DP-CL arranged on the base layer BS, a display element layer EDL arranged on the circuit layer DP-CL, and an encapsulation layer TFL arranged on the display element layer EDL. The encapsulation layer TFL may cover the display element layer EDL.

In one or more embodiments, the electronic device ED may further include a window member WM arranged on the display module DM. The window member WM may include a window WP and an adhesive layer AP, and the adhesive layer AP may be arranged between the display module DM and the window WP. The adhesive layer AP may be an optically clear adhesive film (OCA) or an optically clear adhesive resin layer (OCR). In one or more embodiments, the adhesive layer AP may not be provided.

The window WP may cover an entire outside of the display module DM. The window WP may have a shape corresponding to a shape of the display module DM. In the electronic device ED of one or more embodiments, the window WP may include an optically transparent insulating material. The window WP may be a glass substrate or a polymer substrate. For example, in one or more embodiments, the window WP may be a tempered glass substrate subjected to a strengthening treatment. The window WP may correspond to an uppermost layer of the electronic device ED.

In one or more embodiments, in the electronic device ED, the window member WM may be divided into a transmission portion TA and a bezel portion BZA. The transmissive portion TA may correspond to the active region AA of the display module DM, and the bezel portion BZA may correspond to the peripheral region NAA of the display module DM.

A front surface of the window member WM including the transmission portion TA and the bezel portion BZA corresponds to a front surface of the electronic device ED. Users may view images provided through the transmission portion TA corresponding to the front surface of the electronic device ED.

The bezel portion BZA may define a shape of the transmission portion TA. The bezel portion BZA may be adjacent to the transmission portion TA and may be around (e.g., surround) the transmission portion TA. However, embodiments of the present disclosure are not limited to what is shown, for example, in one or more embodiments, the bezel portion BZA may be adjacent to only one side of the transmission portion TA and a portion thereof may not be provided.

In the electronic device ED of one or more embodiments, a portion of the electronic device ED, which is viewed through the bezel portion BZA may have a lower light transmittance than a portion viewed through the transmission portion TA. In one or more embodiments, in the electronic device ED, the bezel portion BZA may be a portion viewed as having a set or predetermined color.

In the electronic device ED of one or more embodiments, the anti-reflection member RP may include a color filter layer CFL (see FIG. 5) or a polarization layer. For example, the anti-reflection member RP may reduce reflectance of external light incident from the outside, or absorb and block or reduce a portion of (external) light incident from the outside.

The input sensing layer ISL included in the electronic device ED of one or more embodiments may be arranged on the display panel DP. The input sensing layer ISL may detect external inputs applied from the outside. The external inputs may be user inputs. The user inputs may include one or more suitable types (kinds) of external inputs such as a body part of a user, light, heat, pen, or pressure.

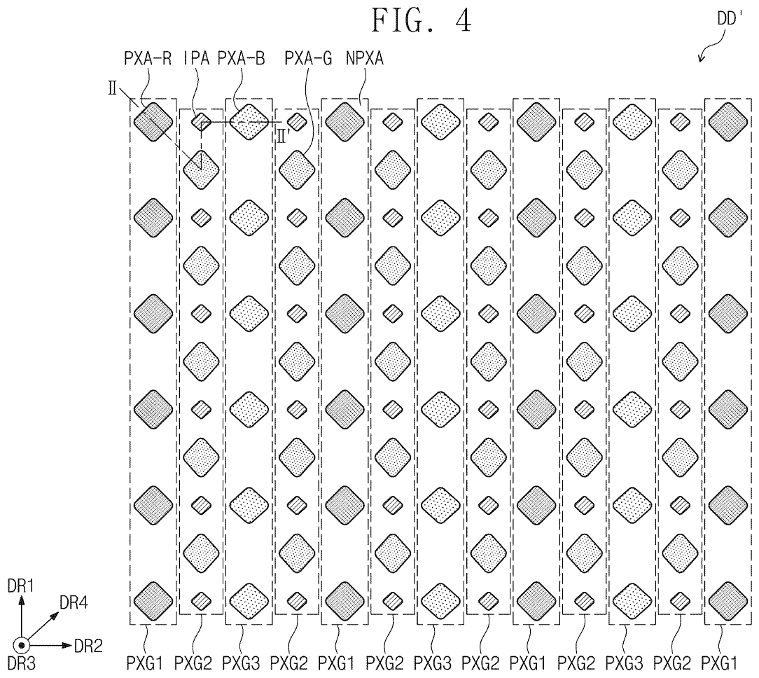

FIG. 4 is a plan view showing a portion of a display module according to one or more embodiments of the present disclosure. FIG. 5 is a cross-sectional view showing a portion of a display module according to one or more embodiments of the present disclosure. FIG. 4 is a plan view showing a region DD′ of FIG. 2, and FIG. 5 is a cross-sectional view showing a portion corresponding to line II-II′ of FIG. 4.

The display module DM according to one or more embodiments may include a plurality of light emitting regions PXA-R, PXA-G, and PXA-B and a light receiving region IPA, which are arranged in the active region AA (see FIG. 2). In one or more embodiments, the display module DM may include a red light emitting region PXA-R to emit red light, a green light emitting region PXA-G to emit green light, and a blue light emitting region PXA-B to emit blue light. In one or more embodiments, The display module DM may include the light receiving region IPA receiving and sensing incident light that is reflected by external objects. A non-light emitting region NPXA may be arranged between the light emitting regions PXA-R, PXA-G, and PXA-B and between the light emitting regions PXA-R, PXA-G, and PXA-B and the light receiving region IPA. Each of the light emitting regions PXA-R, PXA-G, and PXA-B, and the light emitting regions PXA-R, PXA-G, and PXA-B and the light receiving region IPA may be distinguished by the non-light emitting region NPXA. The non-light emitting region NPXA may be around (e.g., surround) each of the light emitting regions PXA-R, PXA-G, and PXA-B and the light receiving region IPA.

In one or more embodiments, the light emitting regions emitting light of different wavelength ranges among the plurality of light emitting regions PXA-R, PXA-G, and PXA-B may have different surface areas from one another. In this case, the surface area may refer to an area if (e.g., when) viewed on a plane defined by the first direction axis DR1 and the second direction axis DR2 (e.g., a plan view).

However, embodiments of the present disclosure are not limited thereto, and the light emitting regions PXA-R, PXA-G, and PXA-B may have substantially the same surface area or different surface area ratios unlike what is shown in FIG. 4. In one or more embodiments, the light emitting regions PXA-R, PXA-G, and PXA-B may be to emit light having a different color except for the red light, the green light, or the blue light, or have a planar shape different from the indicated shape of FIG. 4.

In one or more embodiments, the light receiving region IPA may have a surface area smaller than that of each of the red light emitting region PXA-R, the green light emitting region PXA-G, and the blue light emitting region PXA-B if (e.g., when) viewed on a plane (e.g., in a plan view of the light receiving region IPA). However, embodiments of the present disclosure are not limited thereto, for example, the light receiving region IPA may have a surface area substantially equal to or greater than that of at least any one of the red light emitting region PXA-R, the green light emitting region PXA-G, or the blue light emitting region PXA-B.

Referring to FIG. 4, the red light emitting regions PXA-R may be arranged to be spaced and/or apart (e.g., spaced apart or separated) along the first direction axis DR1 to form (or provide) a first group PXG1. The green light emitting regions PXA-G and the light receiving regions IPA may be alternately arranged along the first direction axis DR1 to form (or provide) a second group PXG2. In addition, the blue light emitting regions PXA-B may be arranged to be spaced and/or apart (e.g., spaced apart or separated) along the first direction axis DR1 to form (or provide) a third group PXG3.

The first group PXG1 to the third group PXG3 may be sequentially arranged in a direction of the second direction axis DR2. Each of the first group PXG1 to the third group PXG3 may be provided in plurality. In one or more embodiments of the present disclosure shown in FIG. 4, the first group PXG1, the second group PXG2, the third group PXG3, and the second group PXG2 constitute one repetition unit along the second direction axis DR2, and these repetition units are repeated and arranged in the second direction axis DR2.

In one or more embodiments, one green light emitting region PXA-G may be arranged to be spaced and/or apart (e.g., spaced apart or separated) from one red light emitting region PXA-R or one blue light emitting region PXA-B in a direction of the fourth direction axis DR4.

In one or more embodiments, the light receiving region IPA is spaced and/or apart (e.g., spaced apart or separated) from each of the light emitting regions PXA-R, PXA-G, and PXA-B, and arranged to be spaced and/or apart (e.g., spaced apart or separated) between the red light emitting region PXA-R and the blue light emitting region PXA-B in the second direction axis DR2. The light receiving region IPA and the green light emitting region PXA-G may be alternately arranged along the first direction axis DR1.

The arrangement structure of the light emitting regions PXA-R, PXA-G, and PXA-B shown in FIG. 4 may be referred to as a PENTILE® structure. PENTILE® is a duly registered trademark of Samsung Display Co., Ltd. However, an arrangement structure of the light emitting regions PXA-R, PXA-G, and PXA-B in an electronic device according to one or more embodiments is not limited to the arrangement structure shown in FIG. 4. For example, in one or more embodiments, the light emitting regions PXA-B, PXA-G, and PXA-R may have a stripe structure in which the red light emitting region PXA-R, the green light emitting region PXA-G, and the blue light emitting region PXA-B are sequentially and alternately arranged. In one or more embodiments, in the stripe arrangement structure, the light receiving region IPA may form (or provide) the same row or the same column along with the green light emitting region PXA-G to form (or provide) a single stripe arrangement. However, in one or more embodiments, the arrangement shape and the arrangement ratio of the light receiving region IPA and the light emitting regions PXA-R, PXA-G, and PXA-B may be different from the above-described arrangement shape.

FIG. 5 is a cross-sectional view showing a portion of the display module according to one or more embodiments of the present disclosure. FIG. 5 is a cross-sectional view showing a portion of the light emitting regions PXA-R, PXA-G, and PXA-B and the light receiving region IPA as an example. For example, FIG. 5 is a cross-sectional view showing a portion of the electronic device ED (FIG. 3) according to one or more embodiments.

The electronic device according to one or more embodiments may include light emitting elements ED-R, ED-G, and ED-B and a light receiving element OPD. The light emitting elements ED-R, ED-G, and ED-B may generate light according to electrical signals. In one or more embodiments, the light receiving element OPD may receive optical signals and convert the optical signals into electrical signals.

Referring to FIG. 5, the display module DM according to one or more embodiments may include a base layer BS, a display element layer EDL arranged on the base layer BS, an input sensing layer ISL arranged on the display element layer EDL, and an anti-reflection member RP arranged on the input sensing layer ISL. The display element layer EDL may include a pixel defining film PDL, and light emitting elements ED-R, ED-G, and ED-B and a light receiving element OPD which are distinguished by a pixel defining film PDL. In one or more embodiments, the anti-reflection member RP may include a color filter layer CFL and an organic planarization layer OCL.

In one or more embodiments, the light emitting elements ED-R, ED-G, and ED-B may include a first light emitting electrode AE-ED, a hole control region HTR, respective emission layers EML-R, EML-G, and EML-B, an electron control region ETR, and a second light emitting electrode CE-ED. In one or more embodiments, the light receiving element OPD may include a first light receiving electrode AE-OPD, a hole control layer HTR, a light receiving layer OPL, an electron control layer ETR, and a second light receiving electrode CE-OPD, which are sequentially stacked in the stated order.

In one or more embodiments, the emission layers EML-R, EML-G, and EML-B included in the light emitting elements ED-R, ED-G, and ED-B, respectively, may be layers including an organic light emitting material and/or an inorganic light emitting material, and/or the like, and thus spontaneously emitting light. The light emitting elements ED-R, ED-G, and ED-B may recombine holes and electrons injected from each electrode in the respective emission layers EML-R, EML-G, and EML-B.

In one or more embodiments, the light receiving layer OPL included in the light receiving element OPD may be a light receiving layer that converts incident light into electrical signals. The light receiving element OPD may separate the provided light into electrons and holes, and may be to transmit the electrons and holes to each respective electrode of the light receiving element OPD.

In one or more embodiments, the light emitting elements ED-R, ED-G, and ED-B may respectively be arranged to overlap the light emitting regions PXA-R, PXA-G, and PXA-B. For example, the emission layers EML-R, EML-G, and EML-B included only in the light emitting elements ED-R, ED-G, and ED-B may be arranged to overlap only the light emitting regions PXA-R, PXA-G, and PXA-B, respectively, and may not overlap the light receiving region IPA.

In one or more embodiments, the light receiving element OPD may be arranged to overlap the light receiving region IPA. For example, the light receiving layer OPL, which is included only in the light receiving element OPD, may be arranged to overlap only the light receiving region IPA and may not overlap the light emitting regions PXA-R, PXA-G, and PXA-B.

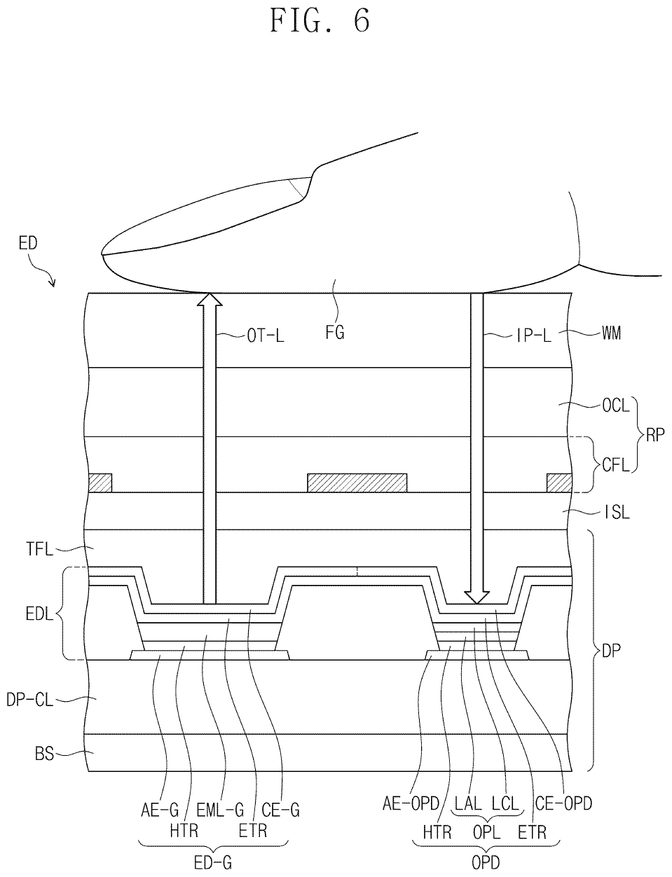

FIG. 6 is a cross-sectional view showing a portion of an electronic device according to one or more embodiments of the present disclosure. FIG. 6 briefly shows operation modes of a light emitting element and a light receiving element. Referring to FIG. 6, in the electronic device ED according to one or more embodiments, light OT-L emitted from the light emitting element ED-G included in the display element layer EDL may be reflected from external objects (e.g., a fingerprint FG in the presented embodiment) and be incident to the light receiving element OPD included in the display element layer EDL as reflected light IP-L. The reflected light IP-L incident on the light receiving element OPD may be light in a visible light range. The light receiving element OPD may receive the incident light and convert the received light into electrical signals to recognize external inputs, thereby changing a driving state of the electronic device ED.

For example, light emitted from the light emitting element ED-G and reflected from external objects is incident on the light receiving element OPD, and the incident light is separated into holes and electrons in the light receiving layer OPL of the light receiving element OPD, and then the holes and the electrons are transmitted to respective electrode AE-OPD and CE-OPD. Accordingly, optical signals are converted into electrical signals.

Referring back to FIG. 5, the electronic device according to one or more embodiments may include a base layer BS in the display module DM. The base layer BS may be a member providing a base surface on which the display element layer EDL is arranged. The base layer BS may be a glass substrate, a metal substrate, a polymer substrate, and/or the like. However, embodiments of the present disclosure are not limited thereto, for example, the base layer BS may be an inorganic layer, an organic layer, or a composite material layer.

The base layer BS may have a multi-layer structure. For example, in one or more embodiments, the base layer BS may have a three-layer structure of a synthetic resin layer, an adhesive layer, and a synthetic resin layer. In one or more embodiments, the synthetic resin layer may include a polyimide-based resin. In one or more embodiments, the synthetic resin layer may include at least one of an acrylate-based resin, a methacrylate-based resin, a polyisoprene-based resin, a vinyl-based resin, an epoxy-based resin, a urethane-based resin, a cellulose-based resin, a siloxane-based resin, a polyamide-based resin, or a perylene-based resin. In the present disclosure, as described herein, a “˜˜based” resin may be considered as including a functional group of “˜˜”.

The circuit layer DP-CL is arranged on the base layer BS. The circuit layer DP-CL may include an insulating layer, a semiconductor pattern, a conductive pattern, a signal line, and/or the like. The insulating layer, a semiconductor layer, and a conductive layer may be formed on the base layer BS through coating or deposition, and subsequently, the insulating layer, the semiconductor layer, and the conductive layer may be selectively patterned through multiple times of a photolithography process. Then, the semiconductor pattern, the conductive pattern, and the signal line included in the circuit layer DP-CL may be formed.

The display element layer EDL may be arranged on the circuit layer DP-CL. The display element layer EDL may include light emitting elements ED-R, ED-G, and ED-B and a light receiving element OPD. For example, the light emitting elements ED-R, ED-G, and ED-B included in the display element layer EDL may include an organic light emitting element, a quantum dot light emitting element, a micro LED light emitting element, or a nano LED light emitting element. However, embodiments of the present disclosure are not limited thereto, for example, the light emitting elements ED-R, ED-G, and ED-B may include one or more suitable embodiments as long as light is generated or an amount of light is controlled or selected according to electrical signals.

In one or more embodiments, the light receiving element OPD may be a light sensor that receives and recognizes light reflected by external objects. In one or more embodiments, the light receiving element OPD may be a light sensor that recognizes light in a visible light range, which is reflected by external objects. In one or more embodiments, the light receiving element OPD may be a biometric sensor that recognizes light reflected from a user's body part, such as fingerprint and vein, and converts light signals into electrical signals.

The display element layer EDL includes the pixel defining film PDL in which openings OP-E and OP-I are defined, and the light emitting elements ED-R, ED-G, and ED-B and the light receiving element OPD may be separated and distinguished with respect to the pixel defining film PDL. In the pixel defining film PDL, provided are a first opening OP-E in which components of the light emitting elements ED-R, ED-G, and ED-B are arranged and a second opening OP-I in which components of the light receiving element OPD are arranged,

The pixel defining film PDL may be arranged on the base layer BS. The pixel defining film PDL may be arranged on the circuit layer DP-CL, and may expose a portion of an upper surface of a first electrode AE. In the presented embodiment of FIG. 5, the light emitting regions PXA-R, PXA-G, and PXA-B and the light receiving region IPA are defined to correspond to the regions of the first electrodes AE-R, AE-G, AE-B, and AE-OPD exposed in the openings OP-E and OP-I.

In one or more embodiments, the pixel defining film PDL may be formed of a polymer resin. For example, the pixel defining film PDL may be formed including a polyacrylate-based resin or a polyimide-based resin. In one or more embodiments, the pixel defining film PDL may be formed by further including an inorganic material in addition to the polymer resin. In one or more embodiments, the pixel defining film PDL may be formed including a light absorbing material, or may be formed including a black pigment or a black dye. The pixel defining film PDL formed including a black pigment or a black dye may implement a black pixel defining film. When forming (or providing) the pixel defining films PDL, carbon black may be utilized as a black pigment or a black dye, but embodiments of the present disclosure are not limited thereto.

In one or more embodiments, the pixel defining film PDL may be formed of an inorganic material. For example, in one or more embodiments, the pixel defining film PDL may be formed including silicon nitride (SiNₓ), silicon oxide (SiOₓ), silicon oxide (SiOₓN_{y}), and/or the like.

The first electrode AE may include a first light emitting electrode AE-ED arranged corresponding to the first opening OP-E, and a first light receiving electrode AE-OPD arranged corresponding to the second opening OP-I. The first light emitting electrode AE-ED may include a first red light emitting electrode AE-R included in a red light emitting element ED-R, a first green light emitting electrode AE-G included in a green light emitting element ED-G, and a first blue light emitting electrode AE-B included in a blue light emitting element ED-B.

A second electrode CE may include a second light emitting electrode CE-ED arranged on the first light emitting electrode AE-ED, and a second light receiving electrode CE-OPD arranged on the first light receiving electrode AE-OPD. The second light emitting electrode CE-ED may include a second red light emitting electrode CE-R included in the red light emitting element ED-R, a second green light emitting electrode CE-G included in the green light emitting element ED-G, and a second blue light emitting electrode CE-B included in the blue light emitting element ED-B. In one or more embodiments, the second electrode CE may be provided as a common layer.

The light emitting elements ED-R, ED-G, and ED-B may respectively include first light emitting electrodes AE-R, AE-G, and AE-B, second light emitting electrodes CE-R, CE-G, and CE-B, and emission layers EML-R, EML-G, and EML-B.

In one or more embodiments, the display element layer EDL may include a red light emitting element ED-R arranged to correspond to the red light emitting region PXA-R and to emit red light, a green light emitting element ED-G arranged to correspond to the green light emitting region PXA-G and to emit green light, and a blue light emitting element ED-B arranged to correspond to the blue light emitting region PXA-B and to emit blue light. The red light emitting element ED-R may include the first red light emitting electrode AE-R and the second red light emitting electrode CE-R, which facing each other, and a red emission layer EML-R arranged between the first red light emitting electrode AE-R and the second red light emitting electrode CE-R. The green light emitting element ED-G may include the first green light emitting electrode AE-G and the second green light emitting electrode CE-G, which face each other, and a green emission layer EML-G arranged between the first green light emitting electrode AE-G and the second green light emitting electrode CE-G, and the blue light emitting element ED-B may include the first blue light emitting electrode AE-B and the second blue light emitting electrode CE-B, which face each other, and a blue emission EML-B arranged between the first blue light emitting electrode AE-B and the second blue light emitting electrode CE-B.

The light receiving element OPD may include a first light receiving electrode AE-OPD, a second light receiving electrode CE-OPD, and a light receiving layer OPL. The first light receiving electrode AE-OPD may be arranged on the base layer BS and may be exposed through the second opening OP-I. Herein, the first electrode constituting the light receiving element OPD may be referred to as the first light receiving electrode AE-OPD. Herein, the second electrode CE constituting the light receiving element OPD may be referred to as the second light receiving electrode CE-OPD.

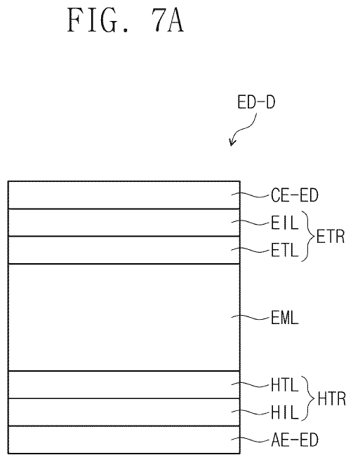

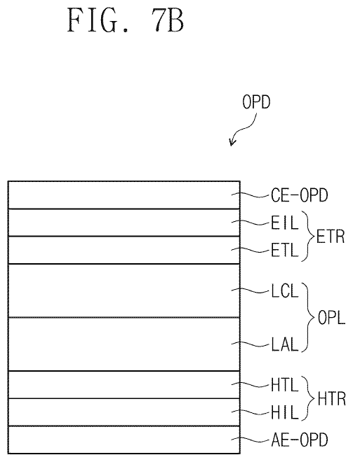

FIGS. 7A and 7B each show cross-sections of a light emitting element and a light receiving element, respectively, as an example. FIG. 7A is a cross-sectional view showing a light emitting element according to one or more embodiments, and FIG. 7B is a cross-sectional view showing a light receiving element according to one or more embodiments.

A light emitting element ED-D according to one or more embodiments shown in FIG. 7A is shown to represent each of the light emitting elements ED-R, ED-G, and ED-B in FIG. 5. Each of the light emitting elements ED-R, ED-G, and ED-B shown in FIG. 5 is different only in the configuration of an emission layer EML from the configuration of the light emitting element ED-D shown in FIG. 7A.

Referring to FIG. 7A, the light emitting element ED-D according to one or more embodiments may include a first light emitting electrode AE-ED, a hole control layer HTR, an emission layer EML, an electron control layer ETR, and a second light emitting electrode CE-ED, which are sequentially stacked in the stated order. In one or more embodiments, the hole control layer HTR may include a hole injection layer HIL and a hole transport layer HTL. The electron control layer ETR may include an electron injection layer EIL and an electron transport layer ETL.

Referring to FIG. 7B, the light receiving element OPD according to one or more embodiments may include a first light receiving electrode AE-OPD, a hole control layer HTR, a light receiving layer OPL, an electron control layer ETR, and a second light receiving electrode CE-OPD, which are sequentially stacked in the stated order. In one or more embodiments, the hole control layer HTR may include a hole injection layer HIL and a hole transport layer HTL. The electron control layer ETR may include an electron injection layer EIL and an electron transport layer ETL. The light receiving layer OPL may generate electrical signals corresponding to incident light. The amount of charge generated in the light receiving layer OPL may rely on the amount and intensity of light incident on the light receiving element OPD. The charge generated in the light receiving layer OPL may alter the electric field between the first light receiving electrode AE-OPD and the second light receiving electrode CE-OPD. The light receiving layer OPL may be arranged between the hole control layer HTR and the electron control layer ETR and may include a light absorption layer LAL and a photoelectric conversion layer LCL. The photoelectric conversion layer LCL may be arranged on the light absorption layer LAL. An interface may be defined between the light absorption layer LAL and the photoelectric conversion layer LCL. The light absorption layer LAL receives external light to form (or provide) excitons, and the excitons may be separated at the interface between the light absorption layer LAL and the photoelectric conversion layer LCL. In this case, electrons may move to the second light receiving electrode CE-OPD through the photoelectric conversion layer LCL, and holes may move to the first light receiving electrode AE-OPD through the light absorption layer LAL. The light receiving element OPD according to one or more embodiments of the present disclosure may obtain fingerprint information of a user through changes in the electric field between the first light receiving electrode AE-OPD and the second light receiving electrode CE-OPD. In one or more embodiments, unlike the light receiving layer OPL shown in FIGS. 5, 6, 7B, 8B, and 9, the light receiving layer OPL may be provided as a single layer formed of a plurality of different materials. In one or more embodiments, the light receiving layer OPL provided as a single layer may include a conjugated polymer. The light receiving layer OPL may include a thiophene-based conjugated polymer, a benzodithiophene-based conjugated polymer, a thieno[3,4-c]pyrrole-4,6-dione (TPD)-based conjugated polymer, a diketo-pyrrole-pyrrole (DPP)-based conjugated polymer, a benzothiadiazole (BT)-based conjugated polymer, and/or the like. However, embodiments of the present disclosure are not limited thereto.

In the light emitting element ED-D and the light receiving element OPD shown in FIGS. 7A and 7B, respectively, the first electrodes AE-ED and AE-OPD may each be formed of a metal material, a metal alloy, or a conductive compound. The first electrode AE may be an anode or cathode. However, embodiments of the present disclosure are not limited thereto. In one or more embodiments, the first electrode AE-ED may be pixel electrodes, and the first electrode AE-OPD may be sensing electrodes. The first electrodes AE-ED and AE-OPD may each independently be a transmissive electrode, a transflective electrode, or a reflective electrode. When each of the first electrodes AE-ED and AE-OPD is the transmissive electrode, the first electrodes AE-ED and AE-OPD may each include a transparent metal oxide such as indium tin oxide (ITO), indium zinc oxide (IZO), zinc oxide (ZnO), and/or indium tin zinc oxide (ITZO). When each of the first electrodes AE-ED and AE-OPD is the transflective electrode or the reflective electrode, the first electrodes AE-ED and AE-OPD may each independently include silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), LiF/Ca, LiF/Al, molybdenum (Mo), titanium (Ti), tungsten (W), a compound thereof, and/or a (e.g., any suitable) mixture thereof (e.g., a mixture of Ag and Mg). In one or more embodiments, the first electrodes AE-ED and AE-OPD may have a multilayer structure including a reflective film or a transflective film formed of one or more of the above-described materials, and a transparent conductive film formed of indium tin oxide (ITO), indium zinc oxide (IZO), zinc oxide (ZnO), indium tin zinc oxide (ITZO), and/or the like. For example, in one or more embodiments, the first electrodes AE-ED and AE-OPD may each have a three-layer structure of ITO/Ag/ITO, but embodiments of the present disclosure are not limited thereto. In one or more embodiments, the first electrodes AE-ED and AE-OPD may include one of the above-described metal materials, a combination of two or more metal materials of (e.g., selected from among) the above-described metal materials, or oxides of the above-described metal materials, but embodiments of the present disclosure are not limited thereto.

The second electrodes CE-ED and CE-OPD may be a common electrode. The second electrodes CE-ED and CE-OPD as the common electrode may be a cathode or an anode but embodiments of the present disclosure are not limited thereto. For example, if (e.g., when) the first electrodes AE-ED and AE-OPD are an anode, the second electrodes CE-ED and CE-OPD may be a cathode, and if (e.g., when) the first electrodes AE-ED and AE-OPD are a cathode, the second electrodes CE-ED and CE-OPD may be an anode.

The second electrodes CE-ED and CE-OPD may be a transmissive electrode, a transflective electrode, or a reflective electrode. When the second electrodes CE-ED and CE-OPD as the common electrode are a transmissive electrode, the second electrodes CE-ED and CE-OPD may be formed of a transparent metal oxide, for example, indium tin oxide (ITO), indium zinc oxide (IZO), zinc oxide (ZnO), indium tin zinc oxide (ITZO), and/or the like. When the second electrodes CE-ED and CE-OPD as the common electrode are a transflective electrode or a reflective electrode, the second electrodes CE-ED and CE-OPD may include Ag, Mg, Cu, Al, Pt, Pd, Au, Ni, Nd, Ir, Cr, Li, Ca, LiF/Ca, LiF/Al, Mo, Ti, Yb, W, a compound thereof, and/or a (e.g., any suitable) mixture thereof (e.g., AgMg, AgYb, or MgYb).

In one or more embodiments, the first electrodes AE-ED and AE-OPD included in the light emitting element ED-D and the light receiving element OPD may be transflective electrodes or reflective electrodes, and the second electrodes CE-ED and CE-OPD may be a transmissive electrode or a transflective electrode. For example, in one or more embodiments, if (e.g., when) the transmissive or transflective second electrodes CE-ED and CE-OPD as the common electrode are included, light reflected from external objects may be easily transmitted to the light receiving element OPD.

In the light emitting element ED-D, the emission layer EML is provided on the hole control layer HTR, which will be described in more detail later. The emission layer EML may have a single layer formed of a single material, a single layer formed of a plurality of materials different from each other, or a multi-layered structure that has a plurality of layers formed of a plurality of materials different from each other.

In the light emitting element ED-D according to one or more embodiments, the emission layer EML may include an organic light emitting material or a quantum dot material. For example, in one or more embodiments, the emission layer EML may include, as an organic light emitting material, an anthracene derivative, a pyrene derivative, a fluoranthene derivative, a chrysene derivative, a dihydrobenzanthracene derivative, or a triphenylene derivative.

In one or more embodiments, in the light emitting element ED-D the emission layer EML may include a host and a dopant. The emission layer EML may include, as a dopant material, an organic fluorescent dopant material, an organic phosphorescent dopant material, a thermally activated delayed fluorescent dopant material, and/or a phosphorescent dopant material of an organometallic complex.

In one or more embodiments, the emission layer EML may include, as a host material, at least one selected from among bis [2-(diphenylphosphino)phenyl]ether oxide (DPEPO), 4,4′-bis(carbazol-9-yl)biphenyl (CBP), 1,3-bis(carbazolyl-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazol-2-yl)benzene (TPBi). However, embodiments of the present disclosure are not limited thereto, for example, tris(8-hydroxyquinolinato)aluminum (Alq₃), poly(N-vinylcarbazole) (PVK), 9,10-di(naphthalen-2-yl)anthracene (ADN), 4,4′,4″-tris(carbazol-9-yl)-triphenylamine (TCTA), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), and/or the like may be included.

In one or more embodiments, the emission layer EML may include, as a suitable dopant material, one or more selected from among styryl derivatives (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi)), perylene and derivatives thereof (e.g., 2,5,8,11-tetra-t-butylperylene (TBP)), pyrene and derivatives thereof (e.g., 1,1-dipyrene, 1,4-dipyrenylbenzene, 1,4-bis(N,N-diphenylamino)pyrene), and/or the like.

In one or more embodiments, the emission layer EML may include, as a suitable phosphorescent dopant material, for example, a metal complex including iridium (Ir), platinum (Pt), osmium (Os), gold (Au), titanium (Ti), zirconium (Zr), hafnium (Hf), europium (Eu), terbium (Tb), or thulium (Tm). In one or more embodiments, iridium(III) bis(4,6-difluorophenylpyridinato-N,C2′) (FIrpic), bis(2,4-difluorophenylpyridinato)-tetrakis(1-pyrazolyl) borate iridium(III) (Fir6), or platinum octaethyl porphyrin (PtOEP) may be utilized as a phosphorescent dopant. However, embodiments of the present disclosure are not limited thereto.

In one or more embodiments, the emission layers EML-R, EML-G, and EML-B of the light emitting elements ED-R, ED-G, and ED-B shown in FIG. 5 may each include different light emitting materials. For example, the emission layers EML-R, EML-G, and EML-B may each include different dopant materials.

The light receiving element OPD according to one or more embodiments includes a polycyclic compound of one or more embodiments of the present disclosure, which will be described in more detail later. The light receiving element OPD according to one or more embodiments may include the polycyclic compound of one or more embodiments to allow charges to readily move in the light receiving layer OPL, and may thus have improved photoelectric efficiency for converting received light into electrical signals.

Herein, the polycyclic compound of one or more embodiments, which will be described in more detail later, may be referred to as a first compound.

The polycyclic compound of one or more embodiments includes a three-ring fused ring, which contains two sulfur(S) atoms as ring-forming atoms, as a central structure (hereinafter referred to as core). The core of one or more embodiments includes two thiophene moieties and one benzene moiety as shown in Formula X.

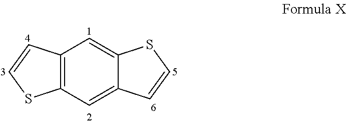

In Formula X, carbon 1 and carbon 2 are carbons forming the benzene moiety, and numbers are assigned to carbon atoms other than the carbons forming the thiophene moiety. In particular, in Formula X, hydrogen atoms having a relatively small volume are substituted on carbons 1 and 2, and thus the core may maintain a planar structure, improving the charge mobility of the polycyclic compound and improving light absorption efficiency. In Formula X, as for carbons 3 to 6, numbers are assigned to some of the carbon atoms constituting the thiophene moiety. The carbons numbered as above are named thiophene carbon 3 to thiophene carbon 6.

In the polycyclic compound of one or more embodiments, a substituted or unsubstituted arylene group having 6 to 60 carbon atoms or a substituted or unsubstituted heteroarylene group having 2 to 60 carbon atoms may be substituted on the thiophene carbon 3 or the thiophene carbon 4 of the core.

In the polycyclic compound of one or more embodiments, a substituted or unsubstituted arylene group having 6 to 60 carbon atoms or a substituted or unsubstituted heteroarylene group having 2 to 60 carbon atoms may be substituted on the thiophene carbon 5 or the thiophene carbon 6 of the core.

In one or more embodiments of the present disclosure, a polycyclic compound is represented by Formula 1.

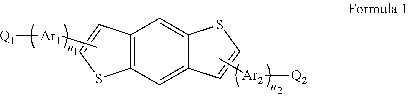

In Formula 1, n₁ and n₂ may each independently be an integer of 0 to 10.

In Formula 1, if (e.g., when) n₁ is 0, Q₁ may be directly bonded to the core of the polycyclic compound of one or more embodiments without a linker indicated as Ar₁. When n₁ is 1 to 10, Ar₁ may be a linker connecting the core and Q₁. When n₁ is an integer of 2 or greater, Ar₁ provided in plurality may all be the same, or at least one of the plurality of Ar₁'s may be different.

In Formula 1, if (e.g., when) n₂ is 0, Q₂ may be directly bonded to the core of the polycyclic compound of one or more embodiments without a linker indicated as Ar₂. When n₂ is 1 to 10, Ar₂ may be a linker connecting the core and Q₂. When n₂ is an integer of 2 or greater, Ar₂ provided in plurality may all be the same, or at least one of the plurality of Ar₂'s may be different.

In Formula 1, Ar₁ and Ar₂ may each independently be a substituted or unsubstituted arylene group having 6 to 60 carbon atoms or a substituted or unsubstituted heteroarylene group having 2 to 60 carbon atoms. For example, in one or more embodiments, Ar₁ and Ar₂ may each independently be a substituted or unsubstituted divalent furan group, a substituted or unsubstituted divalent thiophene group, a substituted or unsubstituted divalent phenyl group, or a substituted or unsubstituted divalent pyridine group, or a substituted or unsubstituted divalent benzothiadiazole group.

In one or more embodiments, in Formula 1, Q₁ and Q₂ may each independently be represented by any one selected from among Formulas Q-1 to Q-7.

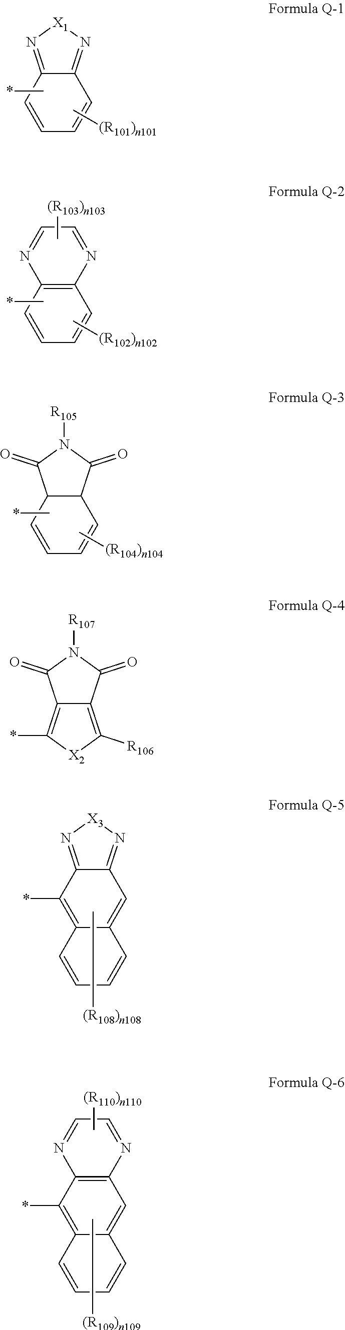

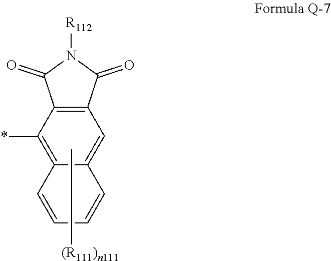

In Formulas Q-1 to Q-3, n₁₀₁, n₁₀₂, and n₁₀₄ may each independently be an integer of 0 to 3.

In Formula Q-1, if (e.g., when) n₁₀₁ is 0, the polycyclic compound of one or more embodiments may be one which is not substituted with R₁₀₁ but in which three hydrogens are substituents. When n₁₀₁ is 3, and R₁₀₁ is hydrogen, the embodiment may be the same as if (e.g., when) n₁₀₁ is 0. When n₁₀₁ is an integer of 2 or greater, R₁₀₁ provided in plurality may all be the same, or at least one of the plurality of R₁₀₁'s may be different. In Formula Q-2, if (e.g., when) n₁₀₂ is 0, the polycyclic compound of one or more embodiments may be one which is not substituted with R₁₀₂ but in which three hydrogens are substituents. When n₁₀₂ is 3, and R₁₀₂ is hydrogen, the embodiment may be the same as if (e.g., when) n₁₀₂ is 0. When n₁₀₂ is an integer of 2 or greater, R₁₀₂ provided in plurality may all be the same, or at least one of the plurality of R₁₀₂'s may be different. In Formula Q-3, if (e.g., when) n₁₀₄ is 0, the polycyclic compound of one or more embodiments may be one which is not substituted with R₁₀₄ but in which three hydrogens are substituents. When n₁₀₄ is 3, and R₁₀₄ is hydrogen, the embodiments may be the same as if (e.g., when) n₁₀₄ is 0. When n₁₀₄ is an integer of 2 or greater, R₁₀₄ provided in plurality may all be the same, or at least one of the plurality of R₁₀₄'s may be different.

In Formulas Q-2 and Q-6, n₁₀₃ and n₁₁₀ may each independently be an integer of 0 to 2.

In Formula Q-2, if (e.g., when) n₁₀₃ is 0, the polycyclic compound of one or more embodiments may be one which is not substituted with R₁₀₃ but in which two hydrogens are substituents. When n₁₀₃ is 2, and R₁₀₃ is hydrogen, the embodiment may be the same as if (e.g., when) n₁₀₃ is 0. When n₁₀₃ is 2, R₁₀₃ provided in plurality may all be the same, or the plurality of Rios's may be different. In Formula Q-6, if (e.g., when) n₁₁₀ is 0, the polycyclic compound of one or more embodiments may be one which is not substituted with R₁₁₀ but in which two hydrogens are substituents. When n₁₁₀ is 2, and R₁₁₀ is hydrogen, the embodiment may be the same as if (e.g., when) n₁₁₀ is 0. When n₁₁₀ is 2, R₁₁₀ provided in plurality may all be the same, or the plurality of R₁₁₀'s may be different.

In Formulas Q-5 to Q-7, n₁₀₈, n₁₀₉, and n₁₁₁ may each independently be an integer of 0 to 5.

In Formula Q-5, if (e.g., when) n₁₀₈ is 0, the polycyclic compound of one or more embodiments may be one which is not substituted with R₁₀₈ but in which five hydrogens are substituents. When n₁₀₈ is 5, and R₁₀₈ is hydrogen, the embodiment may be the same as if (e.g., when) n₁₀₈ is 0. When n₁₀₈ is an integer of 2 or greater, R₁₀₈ provided in plurality may all be the same, or at least one of the plurality of R₁₀₈'s may be different. In Formula Q-6, if (e.g., when) n₁₀₉ is 0, the polycyclic compound of one or more embodiments may be one which is not substituted with R₁₀₉ but in which five hydrogens are substituents. When n₁₀₉ is 5, and R₁₀₉ is hydrogen, the embodiment may be the same as if (e.g., when) n₁₀₉ is 0. When n₁₀₉ is an integer of 2 or greater, R₁₀₉ provided in plurality may all be the same, or at least one of the plurality of R₁₀₉'s may be different. In Formula Q-7, if (e.g., when) n₁₁₁ is 0, the polycyclic compound of one or more embodiments may be one which is not substituted with R₁₁₁ but in which five hydrogens are substituents. When n₁₁₁ is 5, and R₁₁₁ is hydrogen, the embodiment may be the same as if (e.g., when) n₁₁₁ is 0. When n₁₁₁ is an integer of 2 or greater, R₁₁₁ provided in plurality may all be the same, or at least one of the plurality of R₁₁₁'s may be different.

In Formulas Q-1 and Q-2, R₁₀₁ to R₁₀₃ may each independently be hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms. For example, in one or more embodiments, R₁₀₁ to R₁₀₃ may each independently be hydrogen, an unsubstituted methyl group, a cyano group, a nitro group, fluorine, or chlorine.

In Formulas Q-3 to Q-7, R₁₀₄ to R₁₁₂ may each independently be hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms. For example, in one or more embodiments, R₁₀₄ to R₁₁₂ may each independently be hydrogen, an unsubstituted methyl group, a nitro group, an unsubstituted phenyl group, or a phenyl group substituted with fluorine.

In Formulas Q-1 to Q-7, -* is a site linked to Formula 1.

In one or more embodiments, in Formula 1, if (e.g., when) Q₁ and Q₂ are each represented by Formula Q-1, Q₁ and Q₂ may each independently be represented by any one selected from among Formulas R-11 to R-19.

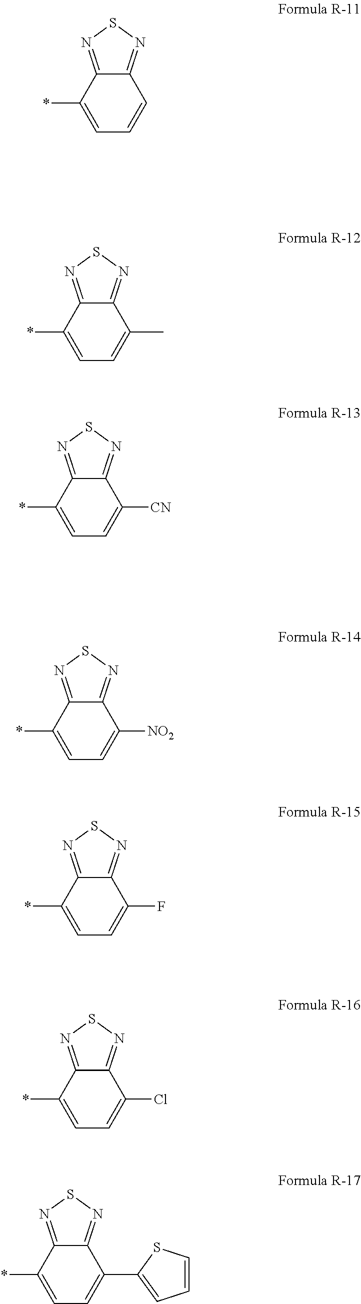

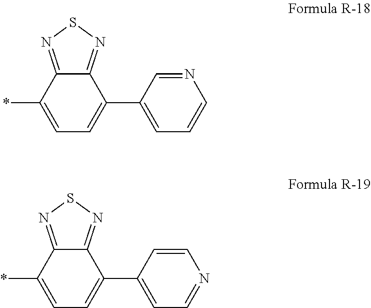 [SMILES: *c1ccc(-c2cccnc2)c2nsnc12 ; *c1ccc(-c2ccncc2)c2nsnc12]

In Formulas R-11 to R-19, -* is a site linked to Formula 1.

In one or more embodiments, in Formula 1, if (e.g., when) Q₁ and Q₂ are each represented by Formula Q-2, Q₁ and Q₂ may each independently be represented by any one selected from among Formulas R-21 to R-24.

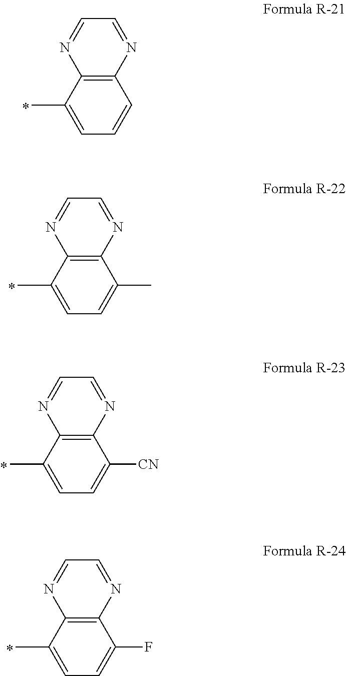

In Formulas R-21 to R-24, -* is a site linked to Formula 1.

In one or more embodiments, in Formula 1, if (e.g., when) Q₁ and Q₂ are each represented by Formula Q-3, Q₁ and Q₂ may each independently be represented by any one selected from among Formulas R-31 to R-34.

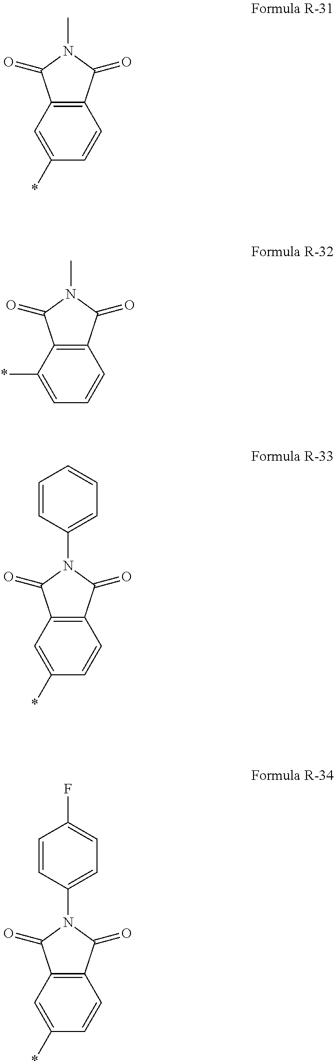 [SMILES: *c1ccc2c(c1)C(=O)N(C)C2=O ; *c1cccc2c1C(=O)N(C)C2=O ; *c1ccc2c(c1)C(=O)N(c1ccccc1)C2=O ; *c1ccc2c(c1)C(=O)N(c1ccc(F)cc1)C2=O]

In Formulas R-31 to R-34, -* is a site linked to Formula 1.

In one or more embodiments, in Formula 1, if (e.g., when) Q₁ and Q₂ are each represented by Formula Q-4, Q₁ and Q₂ may each independently be represented by Formula R-41 or Formula R-42.

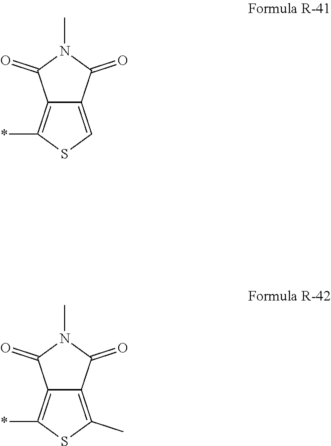 [SMILES: *c1scc2c1C(=O)N(C)C2=O ; *c1sc(C)c2c1C(=O)N(C)C2=O]

In Formulas R-41 and R-42, -* is a site linked to Formula 1.

In one or more embodiments, in Formula 1, if (e.g., when) Q₁ and Q₂ are each represented by Formula Q-5, Q₁ and Q₂ may each independently be represented by Formula R-51 or Formula R-52.

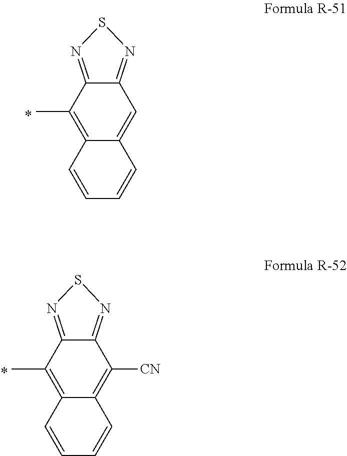 [SMILES: Cc1c2ccccc2cc2nsnc12 ; Cc1c2ccccc2c(C#N)c2nsnc12]

In Formulas R-51 and R-52, -* is a site linked to Formula 1.

In one or more embodiments, in Formula 1, if (e.g., when) Q₁ and Q₂ are each represented by Formula Q-6, Q₁ and Q₂ may each independently be represented by Formula R-61.

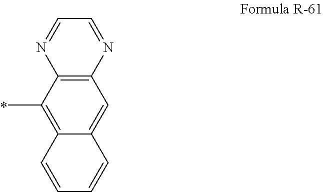

In Formula R-61, -* is a site linked to Formula 1.

In one or more embodiments, in Formula 1, if (e.g., when) Q₁ and Q₂ are each represented by Formula Q-7, Q₁ and Q₂ may each independently be represented by Formula R-71.

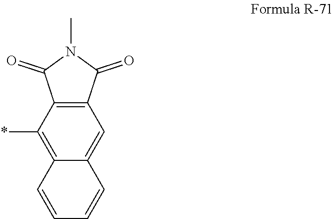

In Formula R-71, -* is a site linked to Formula 1.

The light receiving element OPD of one or more embodiments includes the first compound represented by Formula 1, and thus has higher charge mobility and greater light absorption efficiency than that of a comparative light receiving element including suitable materials in the art. The core of the first compound has a planar structure as two hydrogen atoms are substituted on a benzene ring, and accordingly, the first compound has relatively high charge mobility and improved light absorption efficiency. In one or more embodiments, Q₁ and Q₂ are nitrogen-containing substituents and serve as an electron accepting moiety that accepts electrons. High-energy electrons delivered to Q₁ and Q₂ through incident light are delivered to the core, and the electrons are then donated to another compound. Through this process, the energy of incident light may be delivered to another location, and the delivered energy may be converted into electrical signals. Ar₁ and Ar₂ of the first compound may regulate the conjugation length of the entire first compound to expand absorption wavelength ranges. For example, if (e.g., when) the conjugation length of the first compound is increased by Ar₁ and Ar₂, the first compound may be to absorb green wavelength ranges as well as red wavelength ranges. In one or more embodiments, the light receiving element OPD containing the first compound has greater deposition stability and heat resistance than the light receiving element OPD containing typical materials of the art, and thus has improved purity of a thin film upon substantially continuous deposition. For example, in one or more embodiments, the first compound may have a highest occupied molecular orbital (HOMO) energy of about −5.8 eV to about −5.0 eV, and a lowest unoccupied molecular orbital (LUMO) energy of about −3.9 eV to about −2.7 eV, and may thus effectively absorb light in the green and red wavelength ranges.

In one or more embodiments, the first compound represented by Formula 1 may be represented by any one selected from among Formulas A-1 to A-5.

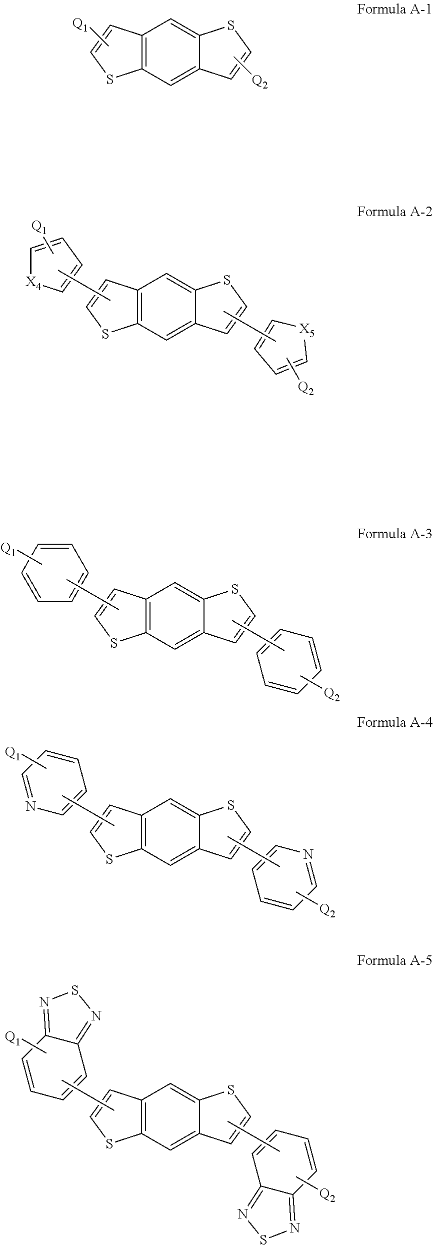

In Formulas A-1 to A-5, X₄ and X₅ may each independently be oxygen, sulfur, selenium, or tellurium. The same descriptions as in Formula 1 may apply to Q₁ and Q₂.

In one or more embodiments, the first compound represented by Formula 1 may be represented by any one selected from among Formulas 1-1 to 1-6.

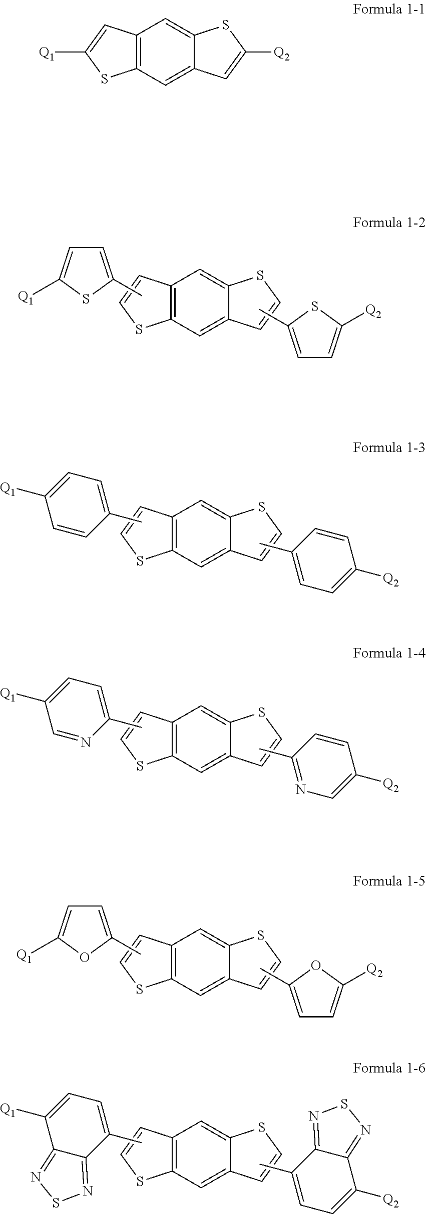

In Formulas 1-1 to 1-6, the same descriptions as in Formula 1 may apply to Q₁ and Q₂.

The polycyclic compound of one or more embodiments may be any one selected from among the compounds shown in Compound Group 1. The light receiving layer OPL of the light receiving element OPD of one or more embodiments may include at least one polycyclic compound selected from among the compounds shown in Compound Group 1 as the first compound.

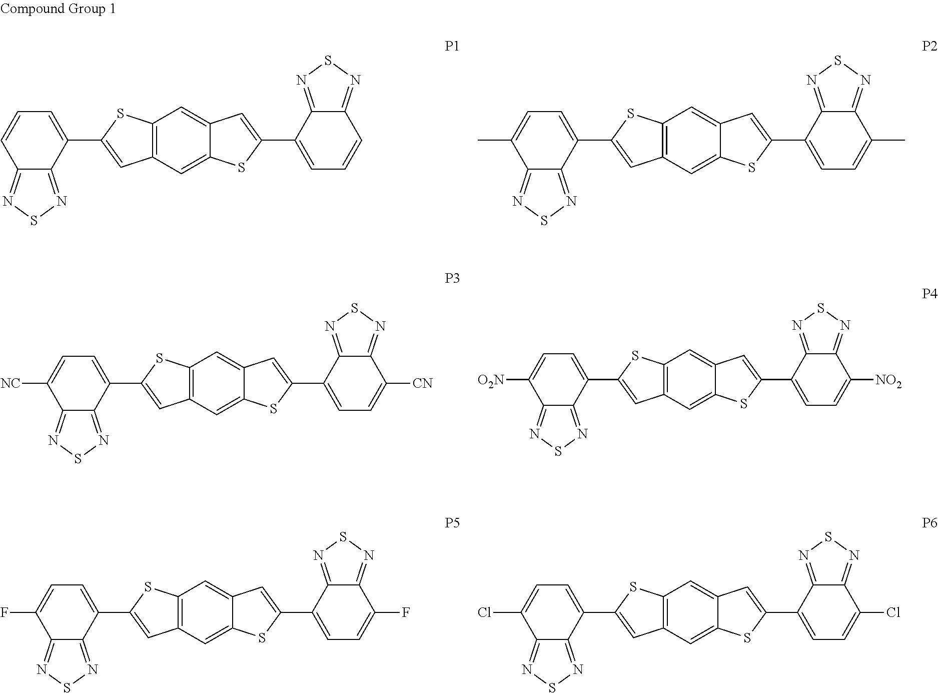

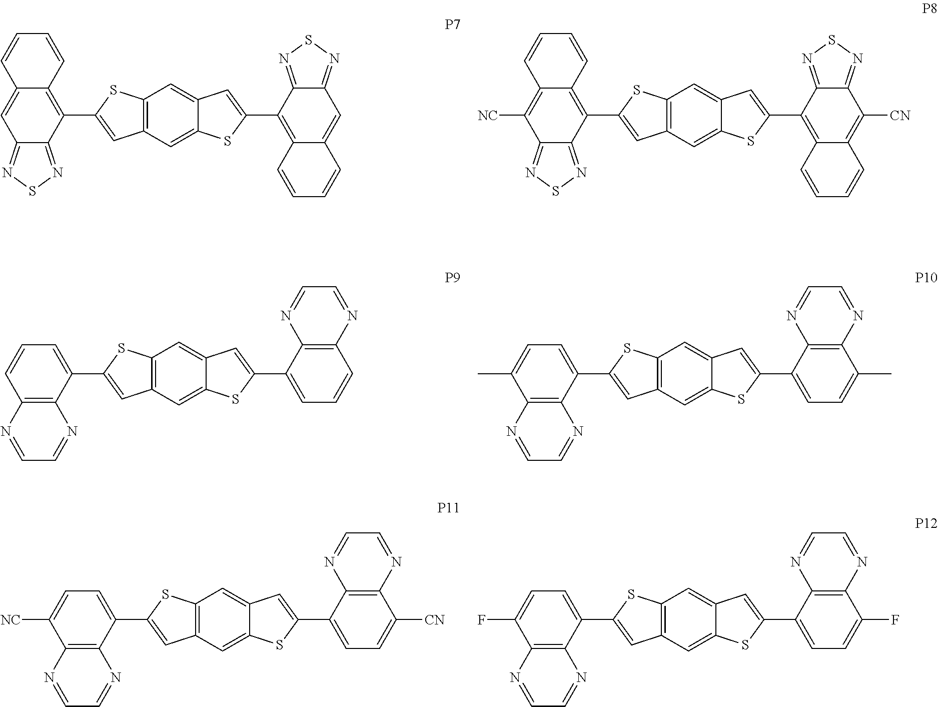

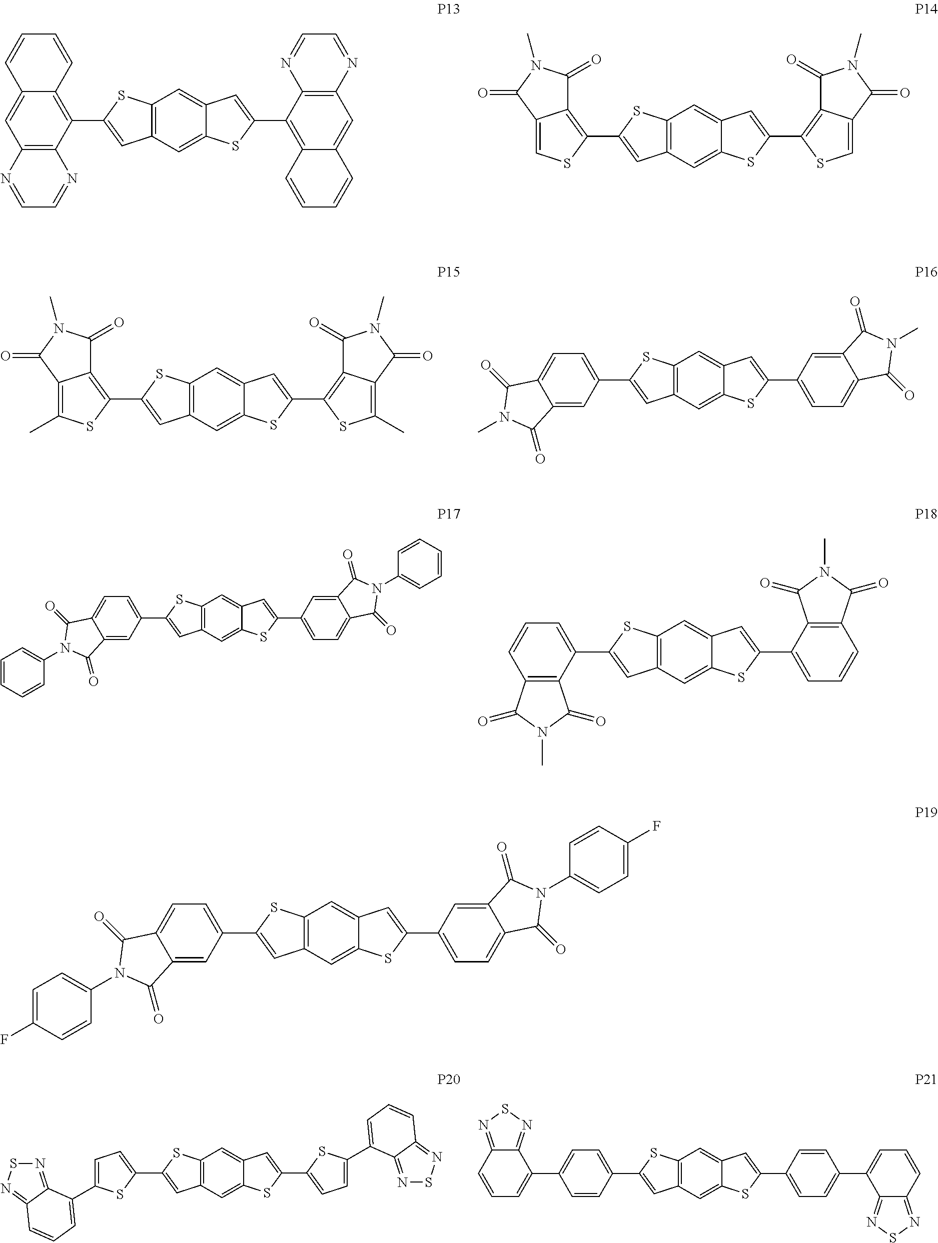

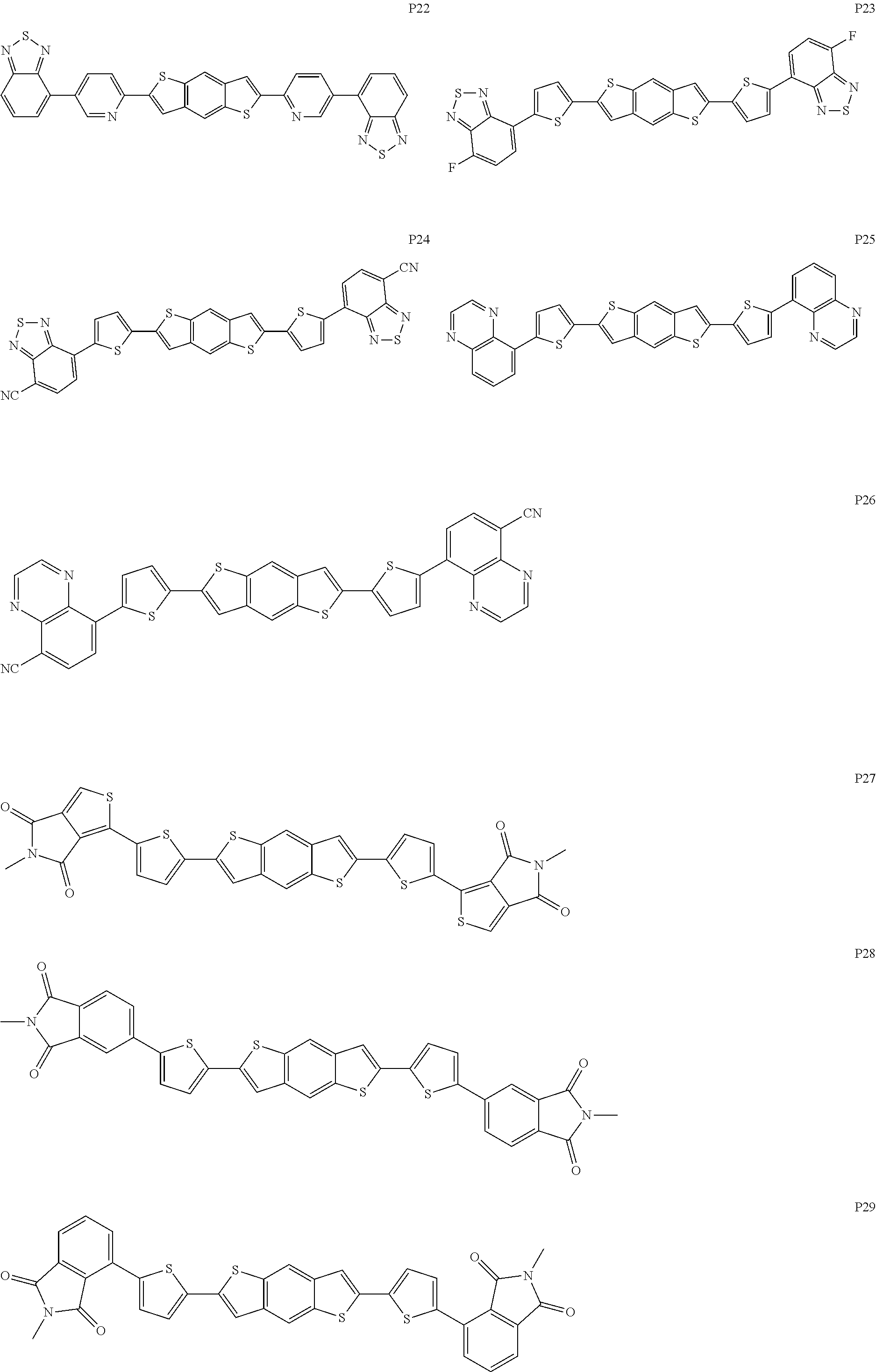

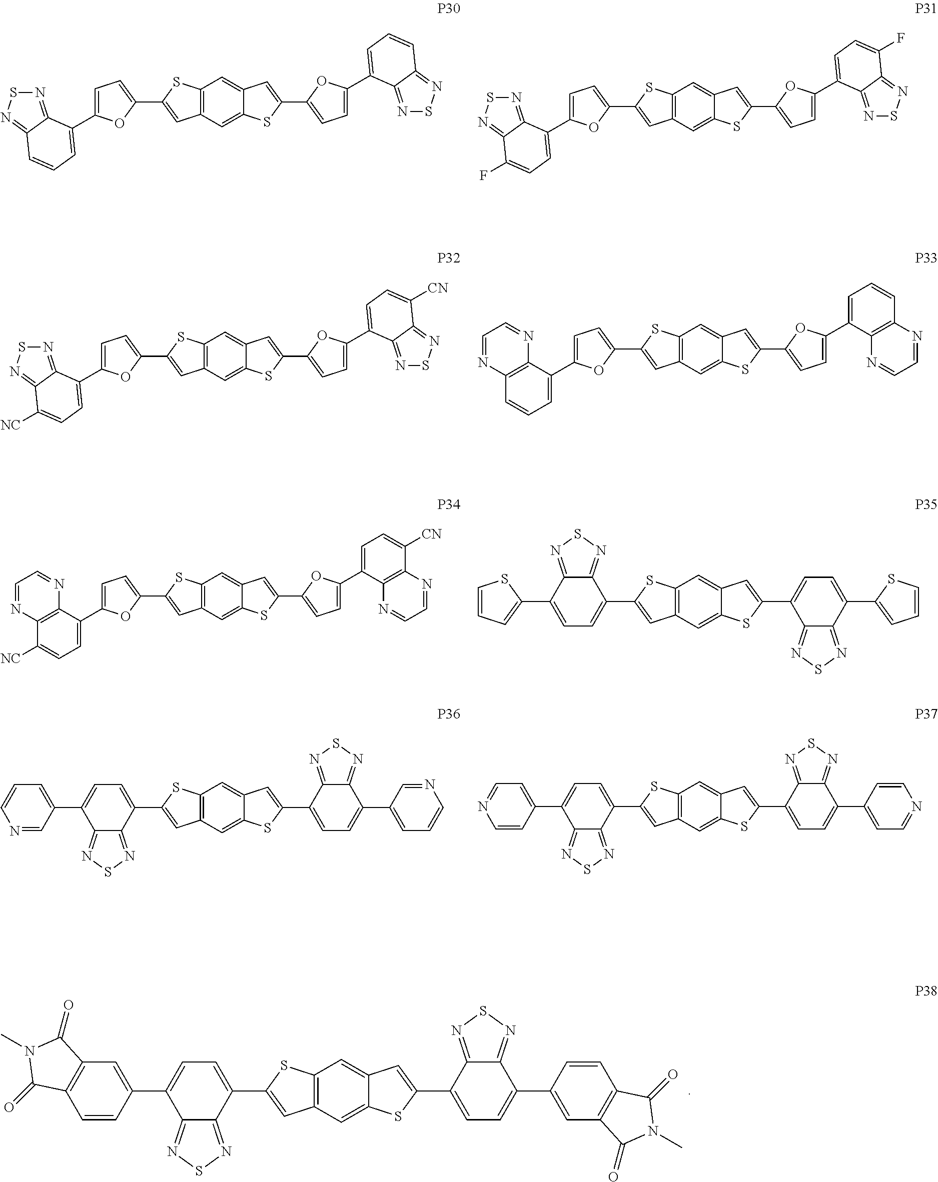

In one or more embodiments, the light receiving layer may further include a second compound represented by Formula 2.

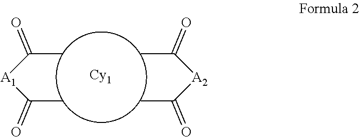

In Formula 2, Cy₁ may be a substituted or unsubstituted aromatic hydrocarbon ring having 6 to 30 carbon atoms. For example, in one or more embodiments, Cy₁ may be unsubstituted naphthalene, unsubstituted biphenyl, unsubstituted benzene, or unsubstituted perylene. A₁ and A₂ may each independently be oxygen or NR₁₁₃. R₁₁₃ may be hydrogen, deuterium, a halogen, a substituted or unsubstituted alkyl group having 1 to 30 carbon atoms, a substituted or unsubstituted aryl group having 6 to 30 carbon atoms, a substituted or unsubstituted heteroaryl group having 2 to 30 carbon atoms, or a substituted or unsubstituted cycloalkyl group having 3 to 30 carbon atoms. For example, in one or more embodiments, R₁₁₃ may be an unsubstituted methyl group, an unsubstituted ethyl group, an unsubstituted t-butyl group, an unsubstituted phenyl group, an unsubstituted tolyl group, a phenyl group substituted with fluorine, a phenyl group substituted with chlorine, an unsubstituted cyanophenyl group, an unsubstituted pyridinyl group, an unsubstituted cyclohexane group, an unsubstituted thiophenyl group, an unsubstituted isopropyl group, an unsubstituted methoxyphenyl group, or an unsubstituted methylthiophenyl group.

The light receiving layer OPL of the light receiving element OPD of one or more embodiments may include at least one polycyclic compound selected from among the compounds shown in Compound Group 2 as the second compound.

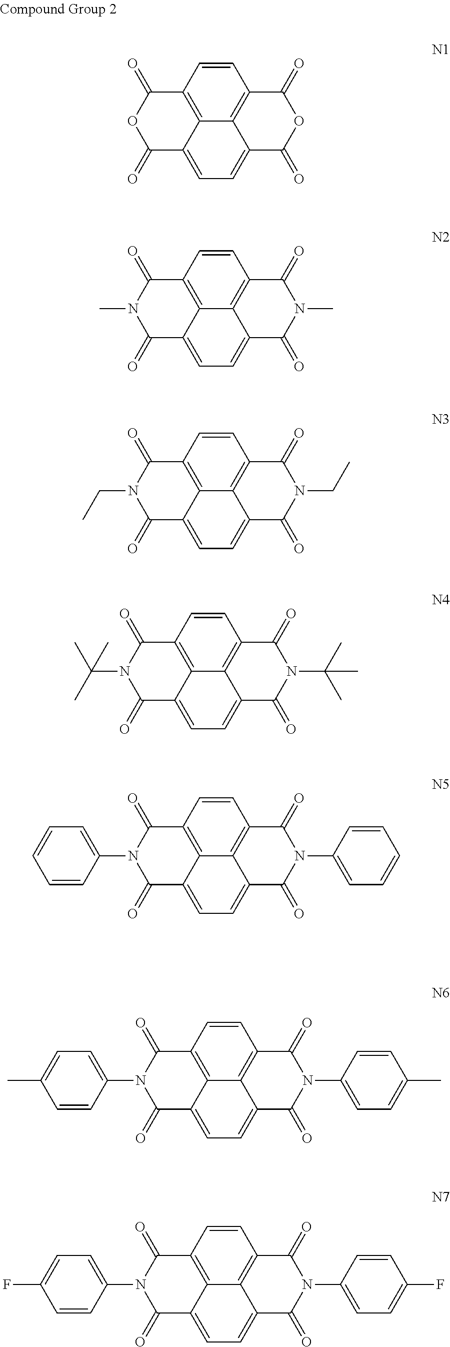

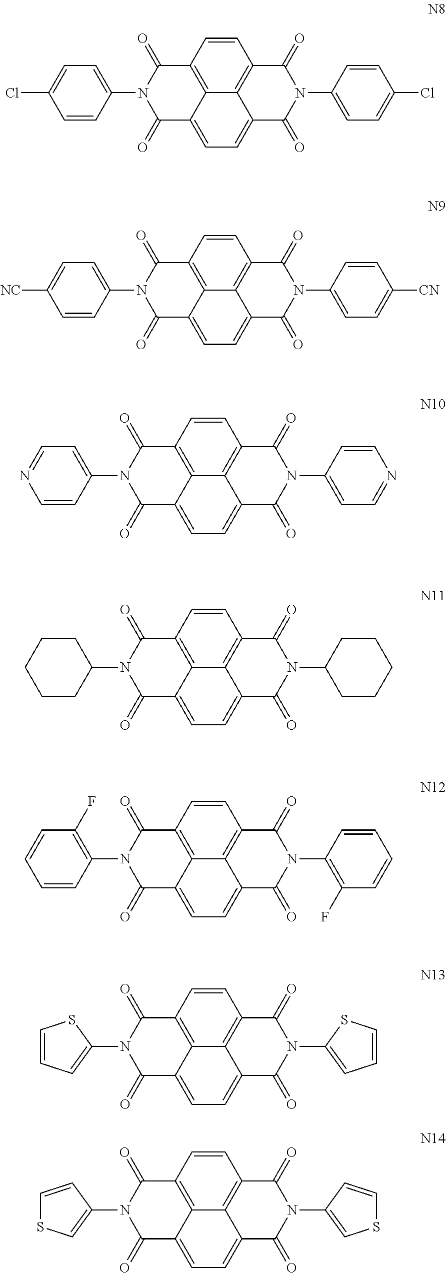

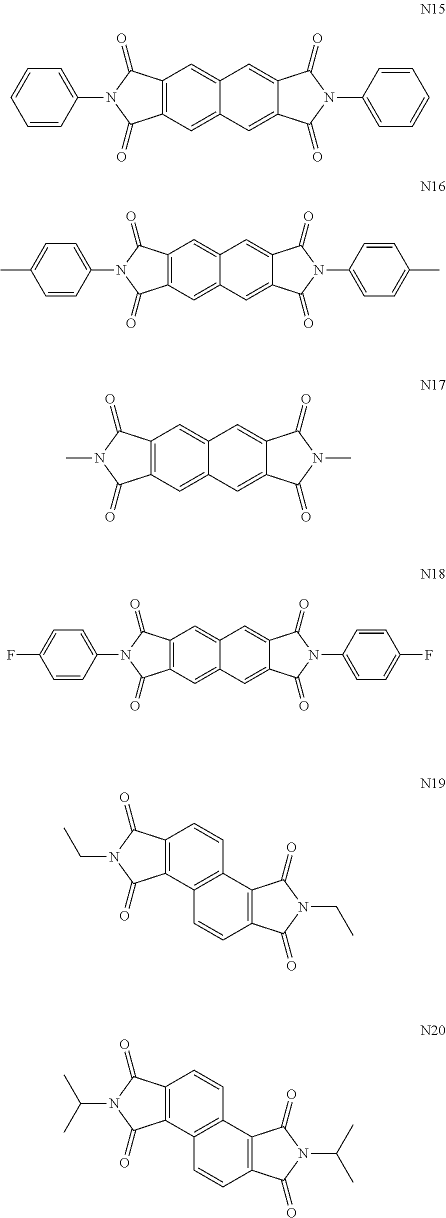

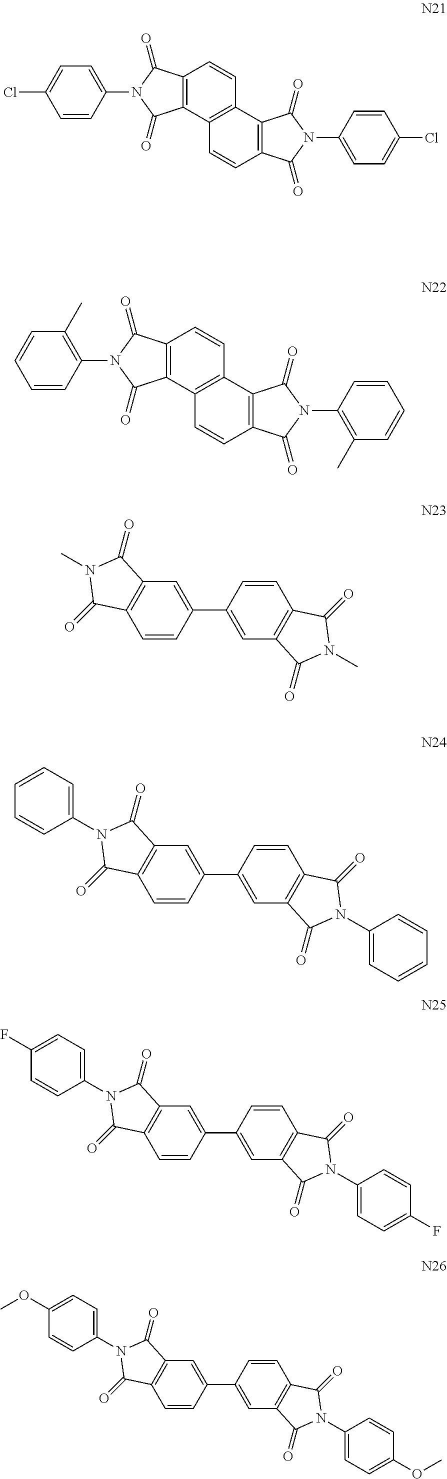

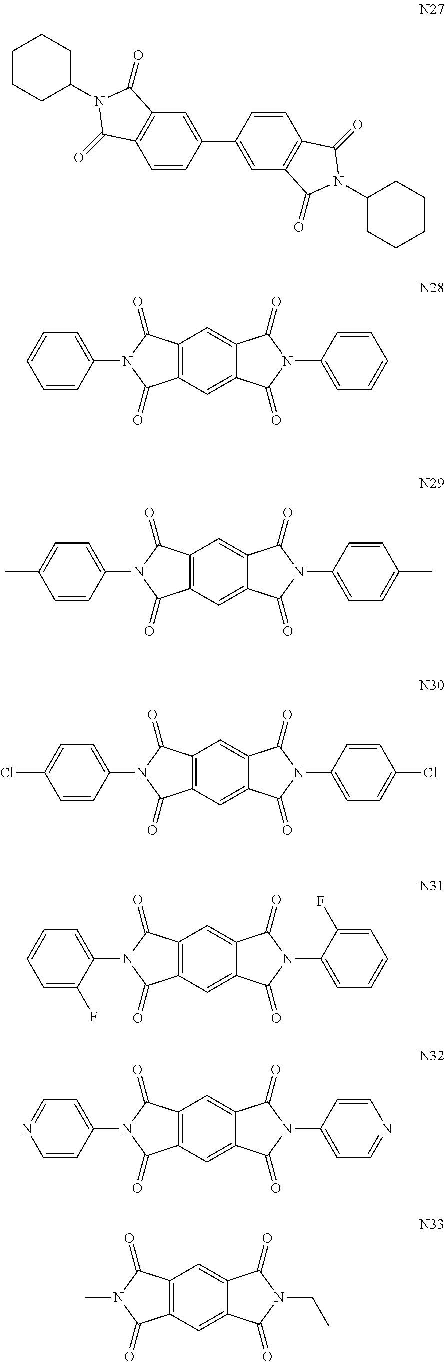

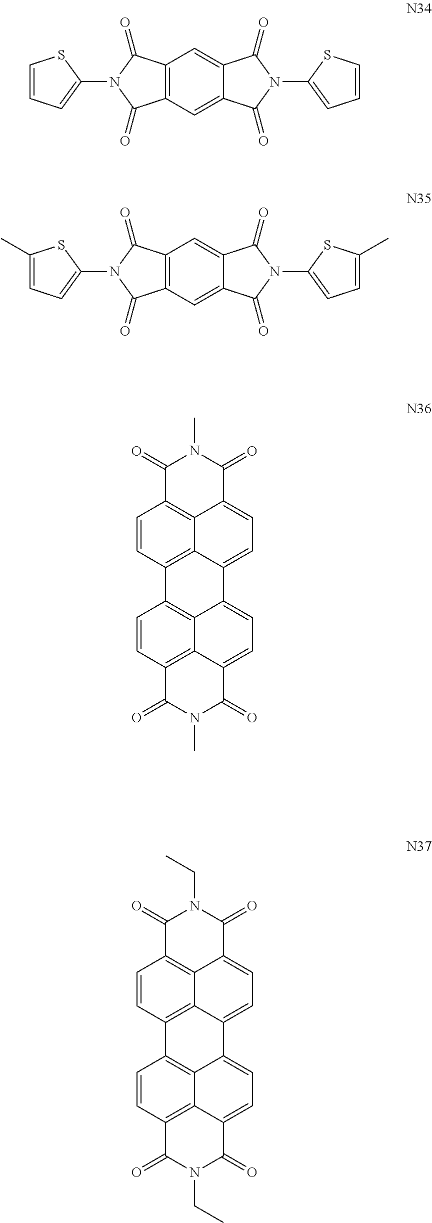

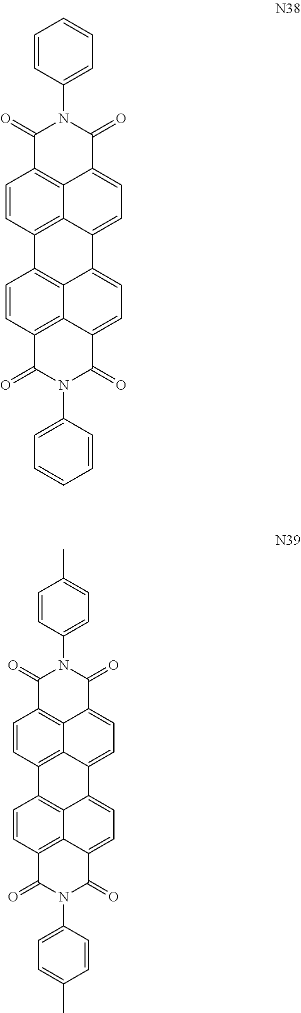 [SMILES: O=C1c2ccc3c4ccc5c6c(ccc(c7ccc(c2c37)C(=O)N1c1ccccc1)c64)C(=O)N(c1ccccc1)C5=O ; Cc1ccc(N2C(=O)c3ccc4c5ccc6c7c(ccc(c8ccc(c3c48)C2=O)c75)C(=O)N(c2ccc(C)cc2)C6=O)cc1]

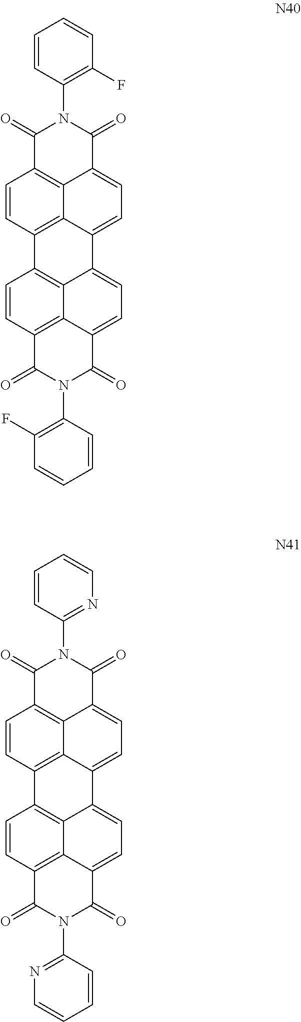 [SMILES: O=C1c2ccc3c4ccc5c6c(ccc(c7ccc(c2c37)C(=O)N1c1ccccc1F)c64)C(=O)N(c1ccccc1F)C5=O ; O=C1c2ccc3c4ccc5c6c(ccc(c7ccc(c2c37)C(=O)N1c1ccccn1)c64)C(=O)N(c1ccccn1)C5=O]

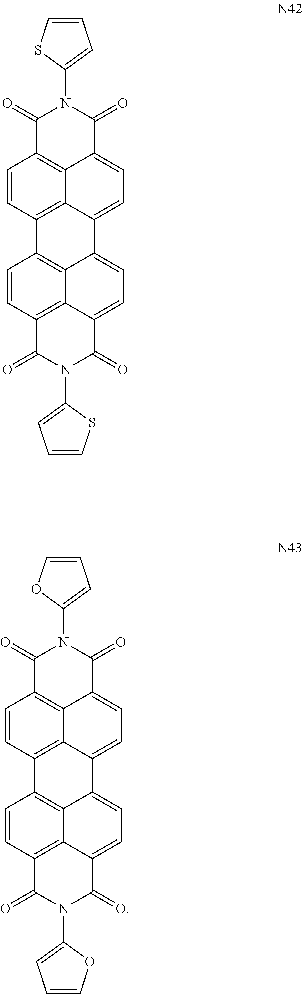 [SMILES: O=C1c2ccc3c4ccc5c6c(ccc(c7ccc(c2c37)C(=O)N1c1cccs1)c64)C(=O)N(c1cccs1)C5=O ; O=C1c2ccc3c4ccc5c6c(ccc(c7ccc(c2c37)C(=O)N1c1ccco1)c64)C(=O)N(c1ccco1)C5=O]

In one or more embodiments, the light receiving layer OPL may include a light absorption layer LAL containing the first compound, and a photoelectric conversion layer LCL containing the second compound. The light absorption layer LAL may serve to absorb light incident on the light receiving element OPD. The photoelectric conversion layer LCL may serve to receive light energy absorbed by the light absorption layer LAL and convert the light energy into electrical signals.

In one or more embodiments, the light receiving layer OPL may include a light absorption layer LAL formed of the first compound, and a photoelectric conversion layer LCL formed of the second compound.

In one or more embodiments, the photoelectric conversion layer LCL may be arranged on the light absorption layer LAL. The photoelectric conversion layer LCL may be arranged between the light absorption layer LAL and the electron control layer ETR, which will be described in more detail later. The light absorption layer LAL may be arranged between the photoelectric conversion layer LCL and the hole control layer HTR.

In one or more embodiments, an interface may be defined between the light absorption layer LAL and the photoelectric conversion layer LCL. The light absorption layer LAL may not form a single layer with the photoelectric conversion layer LCL, but may be a separate layer.

In one or more embodiments, the light receiving element OPD may further include a hole control layer HTR arranged between the first electrode AE (See FIG. 5) and the light receiving layer OPL, and an electron control layer ETR arranged between the light receiving layer OPL and the second electrode CE (See FIG. 5).

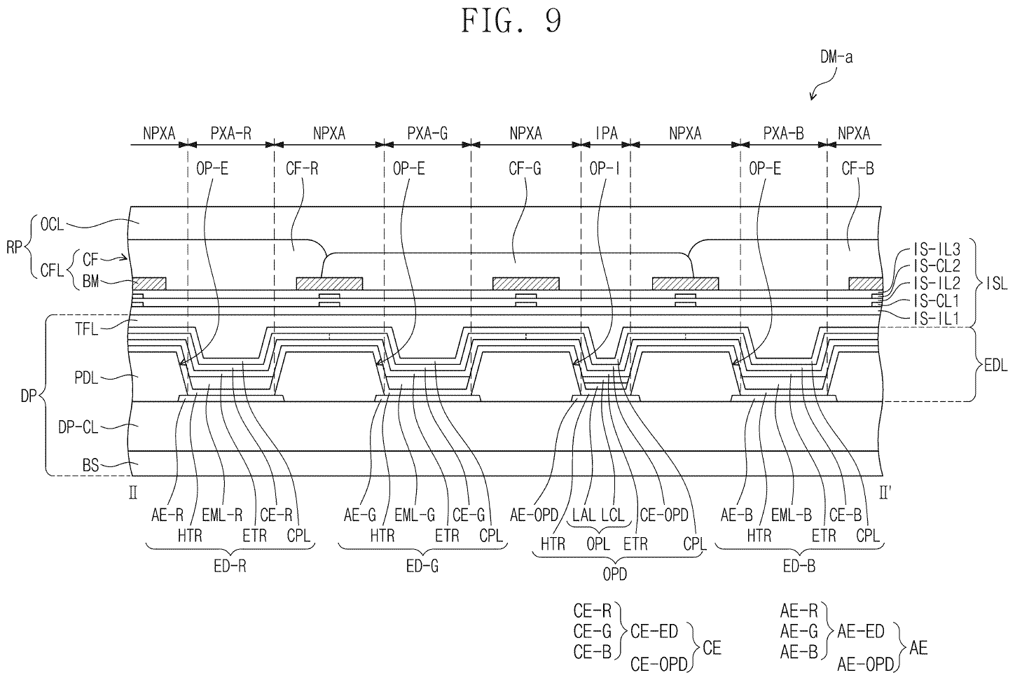

The hole control layer HTR may have a single layer formed of a single material, a single layer formed of a plurality of materials different from each other, or a multi-layered structure that has a plurality of layers formed of a plurality of materials different from each other. For example, in one or more embodiments, the hole control layer HTR may be provided as a single layer instead of being divided into a plurality of layers. The hole control layer HTR may be arranged between the first electrode AE (See FIG. 5) and the light receiving layer OPL. The hole control layer HTR may be arranged between the first electrode AE (See FIG. 5) and the emission layer EML of each of the light emitting elements. In one or more embodiments, in FIG. 5, the hole control layer HTR is shown to be arranged corresponding to the red light emitting region PXA-R, the green light emitting region PXA-G, the blue light emitting region PXA-B, and the light receiving region IPA, but as shown in the description of FIG. 9, which will be described in more detail later, in one or more embodiments, the hole control layer HTR may be provided as a common layer.

Referring to FIGS. 7A and 7B, in one or more embodiments, the hole control layer HTR may include a hole injection layer HIL and a hole transport layer HTL, which are sequentially stacked. In one or more embodiments, the hole control layer HTR may further include at least one of a hole buffer layer, a light emitting auxiliary layer, or an electron blocking layer.

The hole injection layer HIL may include, for example, one or more selected from among a phthalocyanine compound such as copper phthalocyanine, N¹, N¹′-([1,1′-biphenyl]-4,4′-diyl)bis(N¹-phenyl-N⁴, N⁴-di-m-tolylbenzene-1,4-diamine) (DNTPD), 4,4′,4″-[tris(3-methylphenyl)phenylamino] triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium tetrakis(pentafluorophenyl)borate, dipyrazino[2,3-f: 2′,3′-h] quinoxaline-2,3,6,7,10,11-hexacarbonitrile (HAT-CN), and/or the like.

For example, in one or more embodiments, the hole transport layer HTL may include one or more selected from among carbazole-based derivatives such as N-phenyl carbazole and polyvinyl carbazole, fluorene-based derivatives, N,N′-bis(3-methylphenyl)-N,N′-diphenyl-[1,1-biphenyl]-4,4-diamine (TPD), triphenylamine-based derivatives such as 4,4,4″-tris(N-carbazolyl)triphenylamine (TCTA), N,N′-di(naphthalen-1-yl)-N,N′-diphenyl-benzidine (NPB), 4,4-cyclohexylidene bis[N,N-bis(4-methylphenyl]benzenamine] (TAPC), 4,4-bis [N,N′-(3-tolyl)amino]-3,3′-dimethylbiphenyl (HMTPD), 1,3-bis(N-carbazolyl)benzene (mCP), 4,4′-bis(carbazol-9-yl)biphenyl (CBP), and/or the like.

However, embodiments of the present disclosure are not limited thereto, and the hole injection layer HIL and the hole transport layer HTL may include suitable hole injection layer materials or suitable hole transport layer materials in addition to the above-described materials.

In one or more embodiments, the electron control layer ETR may include an electron transport layer ETL and an electron injection layer EIL. However, embodiments of the present disclosure are not limited thereto, and the electron control layer ETR may have a single-layer structure. In one or more embodiments, the electron control layer ETR may further include a hole blocking layer.

In the light emitting element ED-D, the electron control layer ETR may be arranged on the emission layer EML, and in the light receiving element OPD, the electron control layer ETR may be arranged on the light receiving layer OPL.

In one or more embodiments, the electron transport layer ETL may include an anthracene-based compound. However, embodiments of the present disclosure are not limited thereto, and the electron transport layer ETL may include, for example, tris(8-hydroxyquinolinato)aluminum (Alq₃), 1,3,5-tri[(3-pyridyl)-phen-3-yl]benzene, 2,4,6-tris(3-(pyridin-3-yl)biphenyl-3-yl)-1,3,5-triazine, 2-(4-(N-phenylbenzimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate (Bebq₂), 9,10-di(naphthalen-2-yl) anthracene (ADN), 1,3-bis[3,5-di(pyridin-3-yl)phenyl]benzene (BmPyPhB), and/or a (e.g., any suitable) mixture thereof.

In one or more embodiments, the electron injection layer EIL may include halogenated metals such as LiF, NaCl, CsF, RbCl, RbI, CuI, and KI, lanthanide metals such as Yb, or co-deposition materials of a halogenated metal and a lanthanide metal. For example, in one or more embodiments, the electron injection layer EIL may include KI:Yb, RbI:Yb, and/or the like as a co-deposition material. In one or more embodiments, for the electron injection layer EIL, a metal oxide such as Li₂O and/or BaO, or Liq, and/or the like may be utilized, but embodiments of the present disclosure are limited thereto. The electron injection layer EIL may also be formed of a mixture material of an electron transport material and an insulating organo metal salt. The organo metal salt may be a material having an energy band gap of about 4 eV or greater. For example, the organo metal salt may include, for example, metal acetates, metal benzoates, metal acetoacetates, metal acetylacetonates, and/or metal stearates.

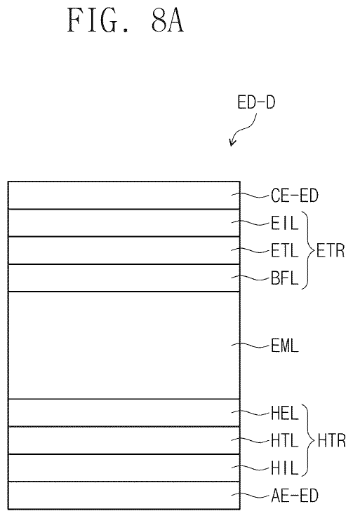

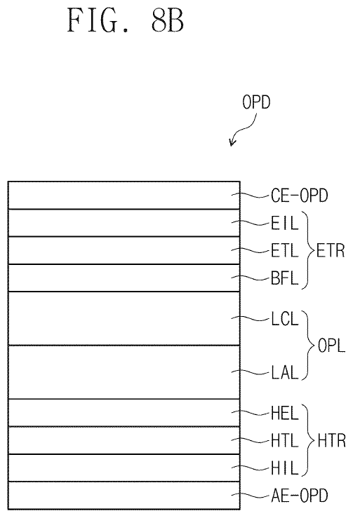

FIGS. 8A and 8B are views of a light emitting element and a light receiving element, respectively, as an example. FIG. 8A is a cross-sectional view showing a light emitting element according to one or more embodiments, and FIG. 8B is a cross-sectional view showing a light receiving element according to one or more embodiments. The light emitting element ED-D according to one or more embodiments shown in FIG. 8A differs from the light emitting element ED-D shown in FIG. 7A in that the light emitting element ED-D according to one or more embodiments shown in FIG. 8A further includes an auxiliary layer HEL and a buffer layer BFL. In one or more embodiments, the light receiving element OPD according to one or more embodiments shown in FIG. 8B differs from the light receiving element OPD shown in FIG. 7B in that the light receiving element OPD according to one or more embodiments shown in FIG. 8B further includes an auxiliary layer HEL and a buffer layer BFL. The light emitting element ED-D shown in FIG. 8A and the light receiving element OPD shown in FIG. 8B may correspond to one or more embodiments of the light emitting elements ED-R, ED-G, and ED-B and the light receiving element OPD of the display element layer EDL of the display module DM shown in FIG. 5.

Referring to FIG. 8A, the light emitting element ED-D according to one or more embodiments may have a structure in which a first light emitting electrode AE-ED, a hole injection layer HIL, a hole transport layer HTL, an auxiliary layer HEL, an emission layer, a buffer layer BFL, an electron transport layer ETL, an electron injection layer EIL, and a second light emitting electrode CE-ED are stacked in the stated order. Referring to FIG. 8B, the light receiving element OPD according to one or more embodiments may have a structure in which a first light receiving electrode AE-OPD, a hole injection layer HIL, a hole transport layer HTL, an auxiliary layer HEL, a light receiving layer OPL, a buffer layer BFL, an electron transport layer ETL, an electron injection layer EIL, and a second light receiving electrode CE-OPD are stacked in the stated order. The hole control layer HTR may include the hole injection layer HIL, the hole transport layer HTL, and the auxiliary layer HEL. The electron control layer ETR may include the buffer layer BFL, the electron transport layer ETL, and the electron injection layer EIL.

The auxiliary layer HEL may include materials utilized in the hole transport layer HTL. For example, in one or more embodiments, the auxiliary layer HEL may include one or more selected from among carbazole-based derivatives such as N-phenyl carbazole and polyvinyl carbazole, fluorene-based derivatives, N,N′-bis(3-methylphenyl)-N,N′-diphenyl-[1,1-biphenyl]-4,4′-diamine (TPD), triphenylamine-based derivatives such as 4,4′,4″-tris(N-carbazolyl)triphenylamine (TCTA), N,N′-di(1-naphthalen-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazolyl)benzene (mCP), 1,3-bis(1,8-dimethyl-9H-carbazol-9-yl)benzene (mDCP), and/or the like.

In the light emitting element ED-D, the auxiliary layer HEL may be utilized as a light emitting auxiliary layer. For example, in the light emitting element ED-D, the auxiliary layer HEL may compensate a resonance distance according to wavelengths of light emitted from the emission layer EML, and may thus increase light emitting efficiency. In one or more embodiments, the auxiliary layer HEL may be provided to be adjusted to different thicknesses according to the wavelength of light emitted from the respective emission layers EML-R, EML-G, and EML-B of the light emitting elements ED-R, ED-G, and ED-B shown in FIG. 5.

In the light receiving element OPD, the auxiliary layer HEL may be utilized as an electron blocking layer. For example, the auxiliary layer HEL may prevent or reduce or reduce the delivery of electrons formed in the light receiving layer OPL to the hole transport layer HTL.

The buffer layer BFL may be utilized as a hole blocking layer. For example, the buffer layer BFL may prevent or reduce or reduce the delivery of holes formed in the light receiving layer OPL to the electron transport layer ETL. In one or more embodiments, the buffer layer BFL may be provided as a common layer. For example, in one or more embodiments, the buffer layer BFL may be provided as a common layer throughout the light emitting element ED-G and the light receiving element OPD. In one or more embodiments, the buffer layer BFL may overlap the entire light emitting regions PXA-R, PXA-G, and PXA-B, light receiving region IPA, and non-light emitting region NPXA.

The buffer layer BFL may include, for example, at least one of 2,9-dimethyl-4,7-diphenyl-1,10-phenanthroline (BCP) or 4,7-diphenyl-1,10-phenanthroline (Bphen). However, embodiments of the present disclosure are not limited thereto.

Referring back to FIG. 5, the respective emission layers EML-R, EML-G, and EML-B of the light emitting elements ED-R, ED-G, and ED-B may each be formed separately in the first opening OP-E. In one or more embodiments, the light receiving layer OPL of the light receiving element OPD may be formed separately in the second opening OP-I.

In one or more embodiments shown in FIG. 5, the electron control layer ETR may be arranged on each of the respective emission layers EML-R, EML-G, and EML-B of the light emitting elements ED-R, ED-G, and ED-B, or be arranged on the light receiving layer OPL of the light receiving element OPD, and may be provided by being separated by the pixel defining film PDL. In one or more embodiments, the electron control layer ETR may be provided as a single common layer. The electron control layer ETR may be provided as a common layer throughout the light emitting elements ED-R, ED-G, and ED-B and the light receiving element OPD. The electron control layer ETR may overlap the entire light emitting regions PXA-R, PXA-G, and PXA-B, light receiving region IPA, and non-light emitting region NPXA.

One or more embodiments may include an encapsulation layer TFL arranged on the light emitting elements ED-R, ED-G, and ED-B and the light receiving element OPD. The encapsulation layer TFL may include at least one inorganic layer and at least one organic layer. For example, in one or more embodiments, the encapsulation layer TFL may include an inorganic layer, an organic layer, and an inorganic layer which are sequentially stacked in the stated order, but the layers forming (or providing) the encapsulation layer TFL are not limited thereto.

The display module DM according to one or more embodiments may include an input sensing layer ISL arranged on the display panel DP. The input sensing layer ISL may be arranged on the display element layer EDL. The input sensing layer ISL may detect external inputs applied from the outside. The external inputs may be user inputs. The user inputs may include one or more suitable types (kinds) of external inputs such as a body part of users, light, heat, pen, or pressure.

In one or more embodiments, the input sensing layer ISL may be formed on the display panel DP through a roll-to-roll process. In these embodiments, the input sensing layer ISL may be expressed as being directly arranged on the display panel DP. Being directly arranged may indicate that a third component is not arranged between the input sensing layer ISL and the display panel DP. For example, a separate adhesive member may not be arranged between the input sensing layer ISL and the display panel DP. For example, the input sensing layer ISL may be directly arranged on the encapsulation layer TFL.

However, embodiments of the present disclosure are not limited thereto, for example, in one or more embodiments, an adhesive member may be further arranged between the input sensing layer ISL and the display panel DP. The input sensing layer ISL may include a lower insulating layer IS-IL1, a first conductive layer IS-CL1, an interlayer insulating layer IS-IL2, a second conductive layer IS-CL2, and an upper insulating layer IS-IL3. In one or more embodiments of the present disclosure, at least one of the lower insulating layer IS-IL1 or the upper insulating layer IS-IL3 may not be provided.

Each of the first conductive layer IS-CL1 and the second conductive layer IS-CL2 may have a structure of a single layer or a structure of multiple layers stacked along the third direction axis DR3. The conductive layer having the multi-layered structure may include at least two or more layers of transparent conductive layers and/or metal layers. The conductive layer having the multi-layered structure may include metal layers having different metals. Each of the transparent conductive layers may include indium tin oxide (ITO), indium zinc oxide (IZO), zinc oxide (ZnO), indium tin zinc oxide (ITZO), PEDOT, a metal nano wire, and/or graphene. Each of the metal layers may include molybdenum, silver, titanium, copper, aluminum, and/or an alloy thereof. For example, in one or more embodiments, each of the first conductive layer IS-CL1 and the second conductive layer IS-CL2 may have a three-layered metal structure, such as, a three-layered structure of titanium/aluminum/titanium. A metal having relatively higher durability and lower reflectivity may be applied to upper/lower layers, and a metal having higher electrical conductivity may be applied to an inner layer.

In one or more embodiments, each of the first conductive layer IS-CL1 and the second conductive layer IS-CL2 includes a plurality of conductive patterns. Hereinafter, the first conductive layer IS-CL1 is described to include first conductive patterns, and the second conductive layer IS-CL2 is described to include second conductive patterns. Each of the first conductive patterns and the second conductive patterns may include sensing electrodes and signal lines connected thereto. The first conductive patterns and the second conductive patterns may be arranged to overlap a light blocking portion BM, which will be described in more detail later. The light blocking portion BM overlaps the first conductive layer IS-CL1 and the second conductive layer IS-CL2 to prevent or reduce reflection of external light by the first conductive layer IS-CL1 and the second conductive layer IS-CL2.

Each of the lower insulating layer IS-IL1, the interlayer insulating layer IS-IL2, and the upper insulating layer IS-IL3 may include an inorganic film or an organic film. In one or more embodiments, the lower insulating layer IS-IL1 and the interlayer insulating layer IS-IL2 may be inorganic films. In one or more embodiments, the upper insulating layer IS-IL3 may include an organic film.

In one or more embodiments, the display module DM may include an anti-reflection member RP arranged on the display panel DP. In one or more embodiments, the anti-reflection member RP may be directly arranged on the input sensing layer ISL. The anti-reflection member RP may include a color filter layer CFL and an organic planarization layer OCL.

The color filter layer CFL may include filter portions CF and a light blocking portion BM. The filter portions CF may include a red filter portion CF-R, a green filter portion CF-G, and a blue filter portion CF-B. The red filter portion CF-R, the green filter portion CF-G, and the blue filter portion CF-B may each be a portion placed to correspond to a red light emitting region PXA-R, a green light emitting region PXA-G, and a blue light emitting region PXA-B, respectively. In one or more embodiments, the green filter portion CF-G may overlap the light receiving region IPA. For example, in one or more embodiments, the green filter portion CF-G may overlap the green light emitting element ED-G and the light receiving element OPD.

The red filter portion CF-R may be to transmit red light, the green filter portion CF-G may be to transmit green light, and the blue filter portion CF-B may be to transmit blue light. Each of the red filter portion CF-R, the green filter portion CF-G, and the blue filter portion CF-B may include a polymer photosensitive resin and a pigment and/or a dye. The red filter portion CF-R may include a red pigment and/or a red dye, the green filter portion CF-G may include a green pigment and/or a green dye, and the blue filter portion CF-B may include a blue pigment and/or a blue dye.

However, embodiments of the present disclosure are not limited thereto, and the blue filter portion CF-B may not include (e.g., may exclude) a pigment and/or a dye. The blue filter portion CF-B may include a polymer photosensitive resin, but may not include (e.g., may exclude) a pigment and/or a dye. The blue filter portion CF-B may be transparent. The blue filter portion CF-B may be formed of a transparent photosensitive resin.

The light blocking portion BM may be arranged on the input sensing layer ISL and overlap borders of neighboring filter portions CF. Edges of the neighboring filter portions CF may overlap one another. For example, in one or more embodiments, the green filter portion CF-G and the red filter portion CF-R are arranged to overlap on the light blocking portion BM, and/or the green filter portion CF-G and the blue filter portion CF-B may be arranged to overlap on the light blocking portion BM. The light blocking portion BM may prevent or reduce light leakage, and separate borders between adjacent color filter portions CF-R, CF-G, and CF-R.

In one or more embodiments, the light blocking portion BM may be a black matrix. The light blocking portion BM may include an organic pigment and/or an organic dye. The light blocking portion BM may be formed including an organic light blocking material and/or an inorganic light blocking material, both (e.g., simultaneously) including a black pigment and/or a black dye. In one or more embodiments, the light blocking portion BM may overlap the pixel defining film PDL. The light blocking portion BM may overlap the pixel defining film PDL placed between the light emitting regions PXA-R, PXA-G, and PXA-B and separating the light emitting regions PXA-R, PXA-G, and PXA-B from the light receiving region IPA.

The organic planarization layer OCL may be arranged on the color filter layer CFL. The organic planarization layer OCL may be arranged on the color filter layer CFL to protect the color filter portions CF-R, CF-G, and CF-B and to planarize an upper surface of the color filter layer CFL. The organic planarization layer OCL may include an organic material such as an acrylic resin or an epoxy resin.

FIG. 9 is a view showing a cross-section of a display module according to one or more embodiments. In describing one or more embodiments with reference to FIG. 9, content overlapping the one described above with reference to FIGS. 1 to 8B will not be described again, and the differences will be mainly described.

Compared with the display module DM according to one or more embodiments shown and described in FIG. 5, a display module DM-a according to one or more embodiments shown in FIG. 9 is different in the configuration of the light emitting elements ED-R, ED-G, ED-B and the light receiving element OPD.

Referring to FIG. 9, compared to one or more embodiments shown in FIG. 5, the hole control layer HTR is different in that a common layer is arranged on throughout the light emitting elements ED-R, ED-G, and ED-B and the light receiving element OPD. The hole control layer HTR may not be patterned and may overlap the entire light emitting regions PXA-R, PXA-G, and PXA-B, light receiving region IPA, and non-light emitting region NPXA.

In one or more embodiments shown in FIG. 9, the display element layer EDL may further include a capping layer CPL. The capping layer CPL may be arranged on the second electrode CE of the light emitting elements ED-R, ED-G, and ED-B and the light receiving element OPD. The capping layer CPL may be arranged between the second electrode CE and the encapsulation layer TFL. The capping layer CPL may be provided as a common layer overlapping all of the pixel defining film PDL, the emission layers EML-R, EML-G, and EML-B, and the light receiving layer OPL.

The capping layer CPL may include a multilayer or a single layer. In one or more embodiments, the capping layer CPL may be an organic layer or an inorganic layer. For example, if (e.g., when) the capping layer CPL includes an inorganic material, the inorganic material may include an alkali metal compound such as LiF, an alkaline earth metal compound such as MgF₂, SiON, SiNx, SiOy, and/or the like.

For example, if (e.g., when) the capping layer CPL includes an organic material, the organic material may include a-NPD, NPB, TPD, m-MTDATA, Alq₃, CuPc, N4, N4, N4′, N4′-tetra(biphenyl-4-yl) biphenyl-4,4′-diamine (TPD15), 4,4′,4″-tris(carbazol-9-yl)triphenylamine (TCTA), and/or the like or may include epoxy resins or acrylates such as methacrylates. However, embodiments of the present disclosure are not limited thereto.

In one or more embodiments, the capping layer CPL may have a refractive index of about 1.6 or greater. In one or more embodiments, the capping layer CPL may have a refractive index of about 1.6 or greater in a wavelength range of about 550 nm to about 660 nm.

Hereinafter, with reference to Examples and Comparative Examples, a polycyclic compound and a light receiving element of one or more embodiments of the present disclosure will be specifically described. In addition, Examples are shown only for the understanding of the present disclosure, and the scope of the present disclosure is not limited thereto.

### 1. Synthesis of Polycyclic Compound

First, a process of synthesizing polycyclic compounds according to one or more embodiments of the present disclosure will be described in more detail by presenting a process of synthesizing Compounds P1, P3, P9, P12, P13, P16, P20, P25, P30, P34, P37, N5, N6, N8, N15, and N16 as an example. In addition, a process of synthesizing polycyclic compounds, which will be described hereinafter, is provided as an example, and thus a process of synthesizing polycyclic compounds according to one or more embodiments of the present disclosure is not limited to Examples.

### (1) Synthesis of Compound P1

2,6-dibromobenzo[1,2-b: 4,5-b′]dithiophene (1.22 g, 3.5 mmol), Benzo-2,1,3-thiadiazole-4-boronic acid (1.28 g, 7.14 mmol), Pd(PPh₃)₄(tetrakis(triphenylphosphine)palladium) (1.16 g, 1.00 mmol), and K₂CO₃ (2.48 g, 17.92 mmol) were dissolved in 120 mL of a tetrahydrofuran (THF)/H₂O (2/1 volume ratio) mixed solution and stirred at 70° C. for 5 hours. After cooling the reaction solution to room temperature, 60 mL of water was added, and the mixture was extracted three times with 80 mL of ethyl ether. The collected organic layer was dried over magnesium sulfate, the solvent was evaporated, and the resulting residue was separated and purified through silica gel tube chromatography to obtain 1.19 g of Compound P1 (yield: 74%). The produced compound was confirmed through ¹H nuclear magnetic resonance (NMR) spectroscopy (CDCl₃, 400 MHZ) and mass spectrometry/fast atom bombardment (MS/FAB).

C₂₂H₁₀N₄S₄: calc. 458.59, found 458.62

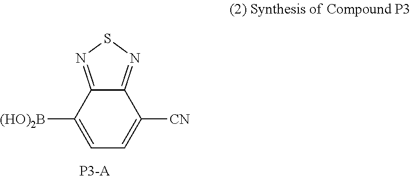 [SMILES: N#Cc1ccc(B(O)O)c2nsnc12]

Compound P3 was synthesized utilizing substantially the same method as the synthesis method of Compound P1, except that Intermediate Compound P3-A was utilized instead of benzo-2,1,3-thiadiazole-4-boronic acid from the synthesis of Compound P1. The produced compound was confirmed through ¹H NMR (CDCl₃, 400 MHz) and MS/FAB.

C₂₄H₈N₆S₄: calc. 508.61, found 508.72

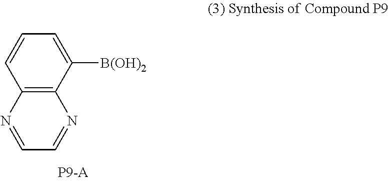 [SMILES: OB(O)c1cccc2nccnc12]

Compound P9 was synthesized utilizing substantially the same method as the synthesis method of Compound P1, except that Intermediate Compound P9-A was utilized instead of benzo-2,1,3-thiadiazole-4-boronic acid from the synthesis of Compound P1. The produced compound was confirmed through ¹H NMR (CDCl₃, 400 MHz) and MS/FAB.

C₂₆H₁₄N₄S₂: calc. 446.55, found 446.61

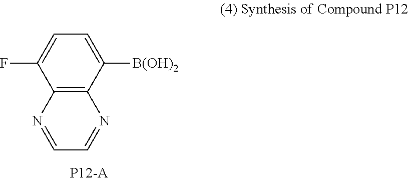 [SMILES: OB(O)c1ccc(F)c2nccnc12]

Compound P12 was synthesized utilizing substantially the same method as the synthesis method of Compound P1, except that Intermediate Compound P12-A was utilized instead of benzo-2,1,3-thiadiazole-4-boronic acid from the synthesis of Compound P1. The produced compound was confirmed through ¹H NMR (CDCl₃, 400 MHz) and MS/FAB.

C₂₆H₁₂F₂N₄S₂: calc. 482.53, found 482.91

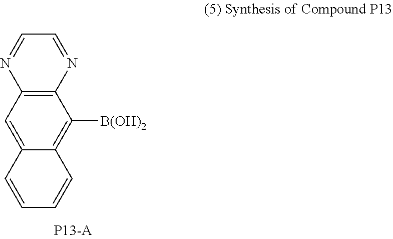 [SMILES: OB(O)c1c2ccccc2cc2nccnc12]

Compound P13 was synthesized utilizing substantially the same method as the synthesis method of Compound P1, except that Intermediate Compound P13-A was utilized instead of benzo-2,1,3-thiadiazole-4-boronic acid from the synthesis of Compound P1. The produced compound was confirmed through ¹H NMR (CDC₃, 400 MHz) and MS/FAB.

C₃₄H₁₈N₄S₂: calc. 546.67, found 546.91

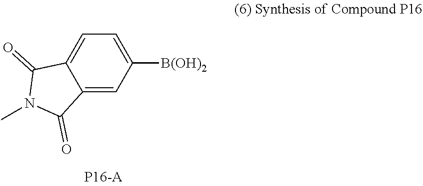

Compound P16 was synthesized utilizing substantially the same method as the synthesis method of Compound P1, except that Intermediate Compound P16-A was utilized instead of benzo-2,1,3-thiadiazole-4-boronic acid from the synthesis of Compound P1. The produced compound was confirmed through ¹H NMR (CDCl₃, 400 MHz) and MS/FAB.

C₂₈H₁₆N₂O₄S₂: calc. 508.57, found 508.94

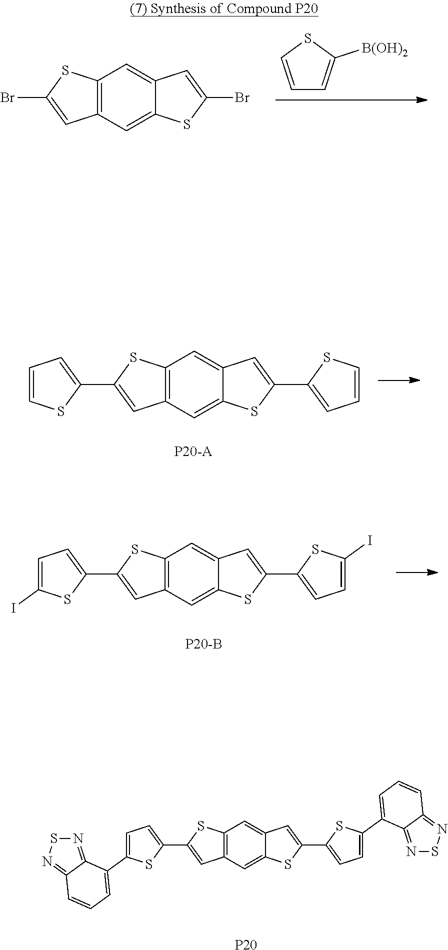

### 1) Synthesis of Intermediate Compound P20-A

Compound P20-A was synthesized utilizing substantially the same method as the synthesis method of Compound P1, except that thiophene-2-ylboronic acid was utilized instead of benzo-2, 1,3-thiadiazole-4-boronic acid from the synthesis of Compound P1. The produced compound was confirmed through ¹H NMR (CDCl₃, 400 MHz) and MS/FAB.

C₁₈H₁₀S₄: calc. 354.52 found 354.64

### 2) Synthesis of Intermediate Compound P20-B

Intermediate Compound P20-A (3.28 g, 9.25 mmol) was dissolved in 50 mL of dehydrated tetrahydrofuran. A 2.76 M n-butyl lithium (n-BuLi) hexane solution (3.4 mL, 9.25 mmol) was added dropwise over 5 minutes at −78° C. and stirred at room temperature for 30 minutes. After lowering the temperature to −78° C. again, iodine (2.5 g, 10 mmol) was added, stirred for 30 minutes, and raised to room temperature. Water was added to terminate the reaction, and then extraction was performed three times with ethyl acetate, and anhydrous magnesium sulfate was added to dry the extracted organic layer. In this case, the product obtained was separated and purified through silica gel tube chromatography (hexane: dichloromethane=1:1 (volume ratio) as an eluent) to obtain 3.98 g of Intermediate Compound P20-B (yield: 71%). The produced compound was confirmed through ¹H NMR (CDCl₃, 400 MHz) and MS/FAB.

C₁₈H₈I₂S₄: calc. 606.31, found 606.45

### 3) Synthesis of Compound P20

Compound P20 was synthesized utilizing the same method as the synthesis method of Compound P1, except that Intermediate Compound P20-B was utilized instead of 2,6-Dibromobenzo[1,2-b: 4,5-b′]dithiophene from the synthesis of Compound P1. The produced compound was confirmed through ¹H NMR (CDCl₃, 400 MHZ) and MS/FAB.

C₃₀H₁₄N₄S₆: calc. 622.83, found 622.90

### (8) Synthesis of Compound P25

Compound P25 was synthesized utilizing substantially the same method as the synthesis method of Compound P9, except that Intermediate Compound P20-B was utilized instead of 2,6-Dibromobenzo[1,2-b: 4,5-b′]dithiophene from the synthesis of Compound P9. The produced compound was confirmed through ¹H NMR (CDCl₃, 400 MHz) and MS/FAB.

C₃₄H₁₈N₄S₄: calc. 610.79, found 610.88

### (9) Synthesis of Compound P30

Compound P30 was synthesized utilizing substantially the same method as the synthesis method of Compound P20, except that furan-2-ylboronic acid was utilized instead of thiophene-2-ylboronic acid from the synthesis of Compound P20. The produced compound was confirmed through ¹H NMR (CDCl₃, 400 MHZ) and MS/FAB.

C₃₀H₁₄N₄O₂S₄: calc. 590.71, found 590.79

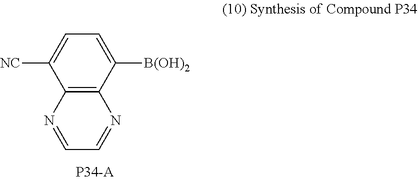 [SMILES: N#Cc1ccc(B(O)O)c2nccnc12]

Compound P34 was synthesized utilizing substantially the same method as the synthesis method of Compound P20, except that furan-2-ylboronic acid was utilized instead of thiophene-2-ylboronic acid and Intermediate Compound P34-A was utilized instead of benzo-2,1,3-thiadiazole-4-boronic acid from the synthesis of Compound P20. The produced compound was confirmed through MS/FAB.

C₃₆H₁₆N₆O₂S₂: calc. 628.68, found 628.77

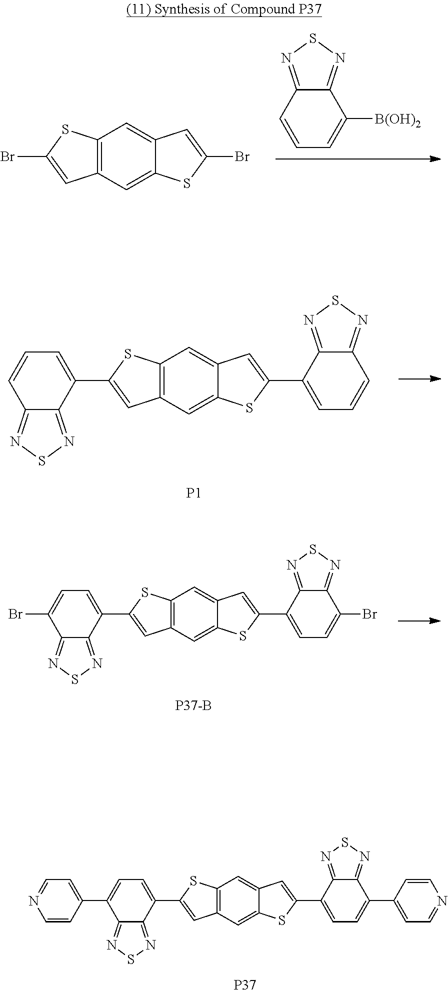

### 1) Synthesis of Intermediate Compound P37-B

Compound P34 was synthesized utilizing substantially the same method as the synthesis method of Compound P20, except that Compound P1 was utilized instead of Intermediate Compound P20-A and bromine was utilized instead of iodine from the synthesis of Compound P20. The produced compound was confirmed through ¹H NMR (CDCl₃, 400 MHZ) and MS/FAB.

C₂₂H₈Br₂N₄S₄: calc. 616.38 found 616.44

### 2) Synthesis of Compound P37

Compound P37 was synthesized utilizing substantially the same method as the synthesis method of Compound P1, except that Intermediate Compound P37-B was utilized instead of 2,6-dibromobenzo[1,2-b: 4,5-b′]dithiophene and pyridine-4-boronic acid was utilized instead of benzo-2, 1,3-thiadiazole-4-boronic acid from the synthesis of Compound P1. The produced compound was confirmed through ¹H NMR (CDCl₃, 400 MHZ) and MS/FAB.

C₃₂H₁₆N₆S₄: calc. 612.76, found 612.91

### (12) Synthesis of Compound N5

1,4,5,8-naphthalenetetracarboxylic dianhydride (2.48 g, 9.25 mmol) and aniline (1.68 g, 18 mmol) were dissolved in dimethylformamide (DMF), heated, and stirred at 150° C. for 2 hours. The precipitate was filtered to synthesize Compound N5. The produced compound was confirmed through ¹H NMR (CDCl₃, 400 MHZ) and MS/FAB.

C₂₆H₁₄N₂O₄: calc. 418.41, found 418.52

### (13) Synthesis of Compound N6

Compound N6 was synthesized utilizing substantially the same method as the synthesis method of Compound N5, except that p-toluidine was utilized instead of aniline from the synthesis of Compound N5. The produced compound was confirmed through ¹H NMR (CDCl₃, 400 MHZ) and MS/FAB.

C₂₈H₁₈N₂O₄: calc. 446.46, found 446.53

### (14) Synthesis of Compound N8

Compound N8 was synthesized utilizing substantially the same method as the synthesis method of Compound N5, except that 4-chloroaniline was utilized instead of aniline from the synthesis of Compound N5. The produced compound was confirmed through ¹H NMR (CDCl₃, 400 MHZ) and MS/FAB.

C₂₈H₁₂Cl₂N₂O₄: calc. 487.29, found 487.33

### (15) Synthesis of Compound N15

Compound N15 was synthesized utilizing substantially the same method as the synthesis method of Compound N5, except that 2,3,6,7-naphthalenetetracarboxylic dianhydride was utilized instead of 1,4,5,8-naphthalenetetracarboxylic dianhydride from the synthesis of Compound N5. The produced compound was confirmed through ¹H NMR (CDCl₃, 400 MHZ) and MS/FAB.

C₂₆H₁₄N₂O₄: calc. 418.41, found 418.49

### (16) Synthesis of Compound N16

Compound N16 was synthesized utilizing substantially the same method as the synthesis method of Compound N5, except that 2,3,6,7-naphthalenetetracarboxylic dianhydride was utilized instead of 1,4,5,8-naphthalenetetracarboxylic dianhydride and p-toluidine was utilized instead of aniline from the synthesis of Compound N5. The produced compound was confirmed through ¹H NMR (CDCl₃, 400 MHZ) and MS/FAB.

C₂₈H₁₈N₂O₄: calc. 446.46, found 446.54

Table 1 shows results from ¹H NMR (CDCl₃, 400 MHZ) results and MS/FAB of Example Compounds.

TABLE 1

| | | MS/FAB | |
|---|---|---|---|
| No. | ¹H NMR (CDCl₃, 400 MHz) | found | calc. |
| Compound | δ = 8.17-8.15 (m, 2H), 7.86-7.75 | 458.62 | 458.59 |
| P1 | (m, 4H), 7.71 (s, 2H), 7.59 (s, 2H) | | |
| Compound | δ = 8.04-7.95 (m, 6H), 7.59 (s, 2H) | 508.72 | 508.61 |
| P3 | | | |
| Compound | δ = 8.74 (s, 4H), 8.02-7.90 (m, 8H), | 446.61 | 446.55 |
| P9 | 7.59 (s, 2H) | | |
| Compound | δ = 8.74 (s, 4H), 8.00-7.95 (m, 4H), | 482.91 | 482.53 |
| P12 | 7.59-7.57 (s, 4H) | | |
| Compound | δ = 8.97-8.95 (m, 2H), 8.74 (s, 4H), | 546.91 | 546.67 |
| P13 | 8.17-8.15 (m, 4H), 7.95 (s, 2H), | | |
| | 7.59-7.52 (m, 6H) | | |
| Compound | δ = 8.78 (s, 2H), 8.30-8.28 (m, 2H), | 508.94 | 508.57 |
| P16 | 8.20-8.18 (m, 2H), 7.95 (s, 2H), 7.59 | | |
| | (s, 2H), 3.05 (s, 6H) | | |
| Compound | δ = 8.17-8.15 (m, 2H), 7.86-7.82 | 622.90 | 622.83 |
| P20 | (m, 4H), 7.71-7.69 (m, 2H), | | |
| | 7.51-7.48 (m, 4H), 7.34-7.31 (m, 2H) | | |
| Compound | δ = 8.74-8.73 (m, 4H), 8.02-8.00 | 610.88 | 610.79 |
| P25 | (m, 2H), 7.90-7.82(m, 6H), 7.51-7.48 | | |
| | (m, 4H), 7.34-7.31 (m, 2H) | | |
| Compound | δ = 8.17-8.15 (m, 2H), 7.95 (s, 2H), | 590.79 | 590.71 |
| P30 | 7.86-7.84 (m, 2H), 7.73-7.71 (m, 2H), | | |
| | 7.59 (s, 2H), 7.08-7.05 (m, 4H) | | |
| Compound | δ = 8.74-8.72 (m, 4H), 8.20-8.12 | 628.77 | 628.68 |
| P34 | (m, 4H), 7.95 (s, 2H), 7.59 (s, 2H), | | |
| | 7.08-7.05 (m, 4H) | | |
| Compound | δ = 8.71 (m, 4H), 8.05-7.95 (m, 10H), | 612.91 | 612.76 |
| P37 | 7.59 (s, 2H) | | |
| Compound | δ = 8.62 (s, 4H), 7.59-7.57 (m, 6H), | 418.52 | 418.41 |
| N5 | 7.45-7.43 (m, 4H) | | |
| Compound | δ = 8.62 (s, 4H), 7.27-7.25 (m, 8H), | 446.53 | 446.46 |
| N6 | 2.32 (s, 6H) | | |
| Compound | δ = 8.62 (s, 4H), 7.43-7.40 (m, 2H) | 487.33 | 487.29 |
| N8 | | | |
| Compound | δ = 8.92 (s, 4H), 7.58-7.55 (m, 6H), | 418.49 | 418.41 |
| N15 | 7.43-7.40(m, 4H) | | |
| Compound | δ = 8.98 (s, 4H), 7.27-7.25 (m, 8H), | 446.54 | 446.46 |
| N16 | 2.32 (s, 6H) | | |

### Preparation of Light Receiving Element

An ITO glass substrate (corning, 15 Ω/cm², 1200 Å) was cut to a size of about 50 mm×50 mm×0.7 mm, subjected to ultrasonic cleaning with isopropyl alcohol and pure water for 5 minutes each respectively and ultraviolet irradiation for 30 minutes, and then exposed to ozone for cleaning to form (or provide) the ITO glass substrate in a vacuum deposition apparatus.

First, on an upper portion of the substrate was vacuum deposited with 2-TNATA to form (or provide) a hole injection layer having a thickness of 600 Å, and then 4,4′-bis[N-(1-naphthyl)-N-phenylamino]biphenyl (hereinafter referred to as NPB), a hole transport material as a hole transport compound, was subjected to vacuum deposition to form (or provide) a hole transport layer having a thickness of 300 Å.

A green auxiliary layer (or red auxiliary layer) was deposited to have a thickness of 300 Å on an upper portion of the hole transport layer, and then a light absorption layer was deposited to have a thickness of 250 Å, utilizing P-type or kind compounds of Examples or Comparative Examples. A photoelectric conversion layer was deposited to have a thickness of 250 Å on an upper portion of the light absorption layer, utilizing N-type or kind compounds of Examples or Comparative Examples. Alq₃ was subjected to deposition on an upper portion of the photoelectric conversion layer to form (or provide) an electron transport layer having a thickness of 300 Å, and then LiF, an alkali metal halide, was subjected to deposition on an upper portion of the electron transport layer to form (or provide) an electron injection layer having a thickness of 10 Å, and Al was subjected to vacuum deposition to form (or provide) a LiF/Al electrode having a thickness of 3000 Å (negative electrode), thereby obtaining an organic light receiving element.

For the obtained element, the characteristics of the organic light receiving element were measured and evaluated. The characteristics are evaluated based on external quantum efficiency (EQE) according to the wavelength of a light receiving element. The external quantum efficiency is measured by absorbing external light by wavelength and converting the light into current (EQE, %), and Xenon Lamp was utilized as a light source. In particular, external quantum efficiency values were measured in green light (530 nm) and red light (630 nm). Meanwhile, the structures of the compounds utilized for element manufacturing are as follows.

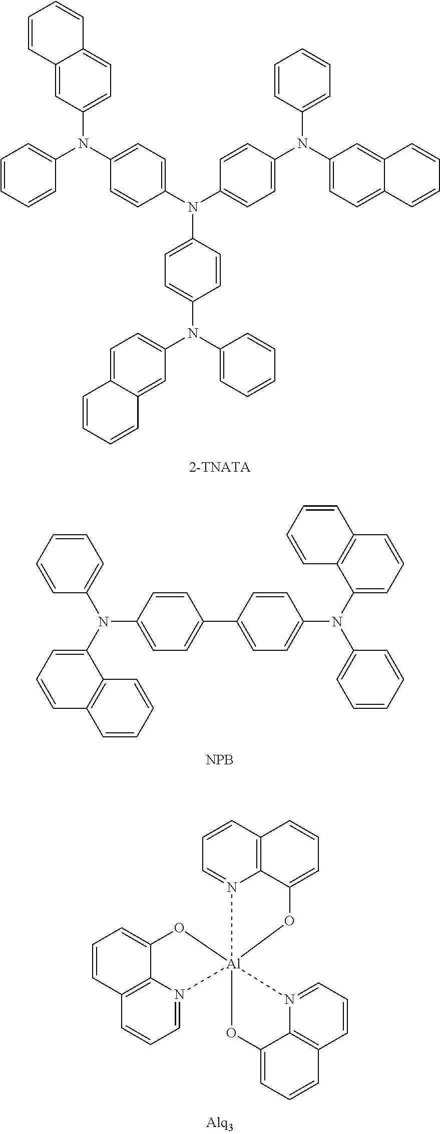

### Evaluation of Characteristics of Light Receiving Element

Table 2 shows a comparison of light receiving elements each respectively containing the above-described Example Compounds P1 and N5, P3 and N5, P9 and N6, P12 and N6, P13 and N6, P16 and N8, P20 and N6, P25 and N6, P30 and N8, P34 and N15, and P37 and N16, with light receiving elements each respectively containing Comparative Example Compounds Sub PC and C₆₀ (Fullerene), and SubNC and C₆₀ (Fullerene) in external quantum efficiency (EQE (%)) and maximum absorption wavelength (λₘₐₓ (nm)). Greater values of external quantum efficiency indicate greater conversion of energy applied to an element from the outside, and thus light absorption efficiency of the element may be evaluated as improved. Meanwhile, the structures of Example Compounds and Comparative Example Compounds utilized for element manufacturing are as follows.

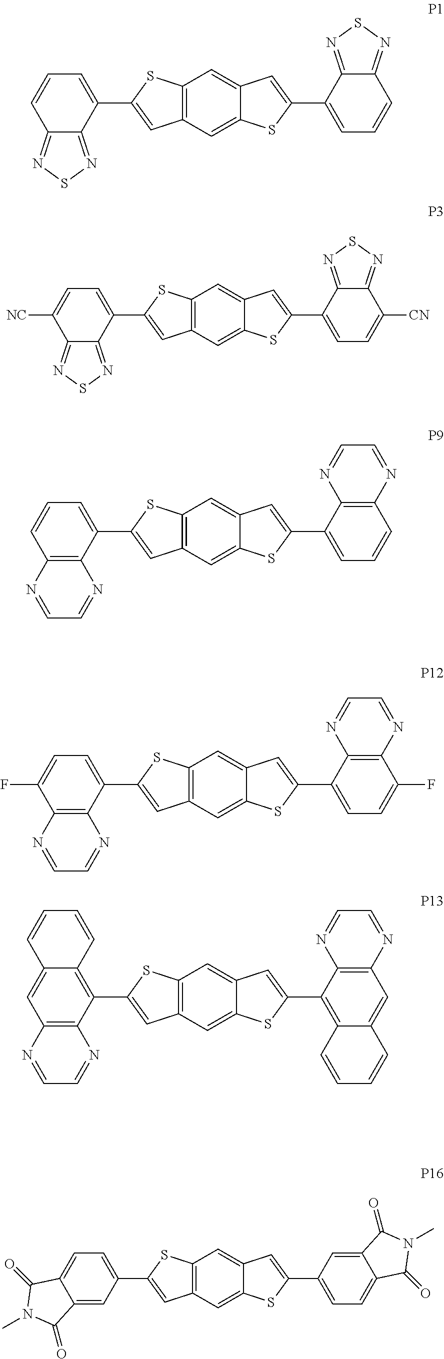

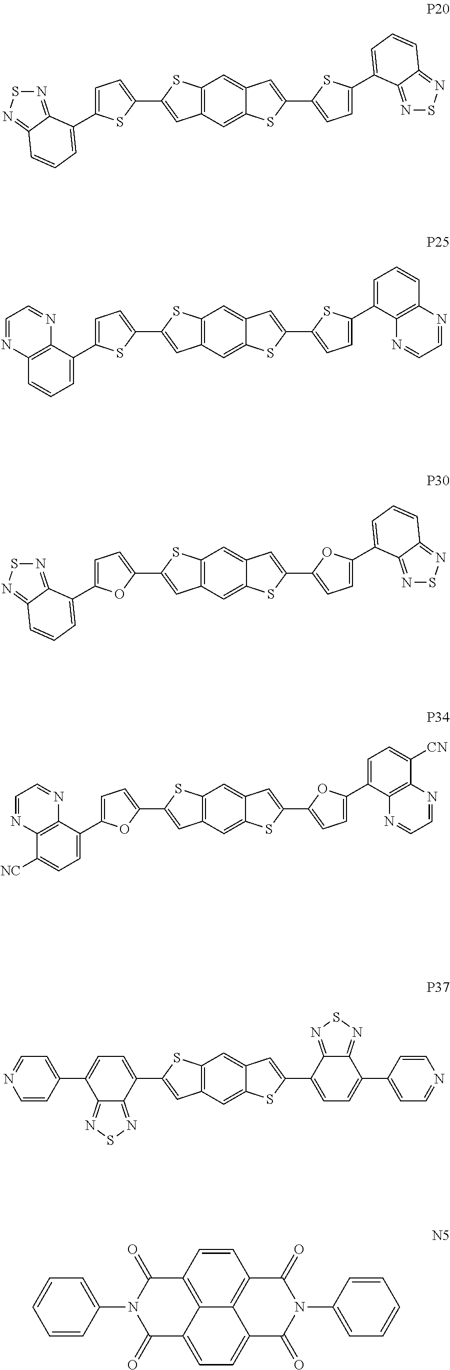 [SMILES: c1cc(-c2ccc(-c3cc4cc5sc(-c6ccc(-c7cccc8nsnc78)s6)cc5cc4s3)s2)c2nsnc2c1 ; c1cc(-c2ccc(-c3cc4cc5sc(-c6ccc(-c7cccc8nccnc78)s6)cc5cc4s3)s2)c2nccnc2c1 ; c1cc(-c2ccc(-c3cc4cc5sc(-c6ccc(-c7cccc8nsnc78)o6)cc5cc4s3)o2)c2nsnc2c1 ; N#Cc1ccc(-c2ccc(-c3cc4cc5sc(-c6ccc(-c7ccc(C#N)c8nccnc78)o6)cc5cc4s3)o2)c2nccnc12 ; c1cc(-c2ccc(-c3cc4cc5sc(-c6ccc(-c7ccncc7)c7nsnc67)cc5cc4s3)c3nsnc23)ccn1 ; O=C1c2ccc3c4c(ccc(c24)C(=O)N1c1ccccc1)C(=O)N(c1ccccc1)C3=O]

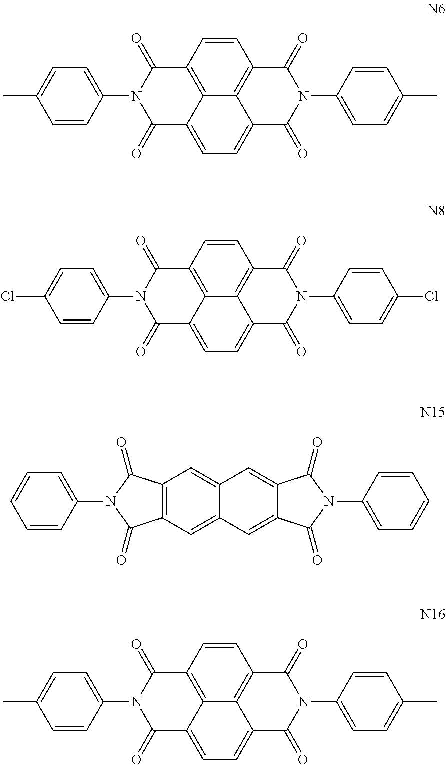

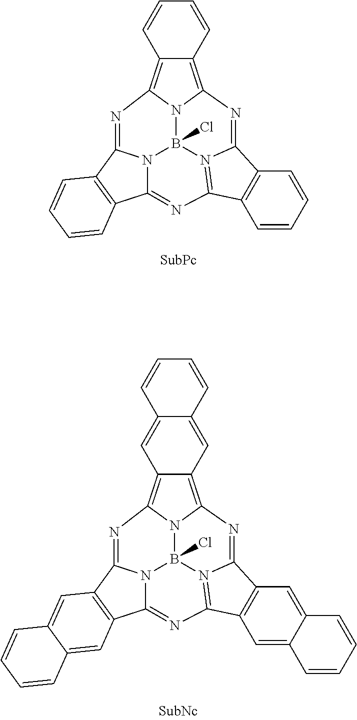

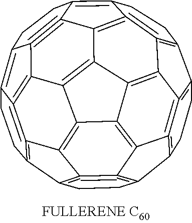

TABLE 2

| Light | P-type | N-type or | | |
|---|---|---|---|---|
| receiving | or kind | kind | EQE | λₘₐₓ |
| element | compound | compound | (%) | (nm) |
| Example 1 | P1 | N5 | 38 | 530 |
| Example 2 | P3 | N5 | 35 | 525 |
| Example 3 | P9 | N6 | 37 | 528 |
| Example 4 | P12 | N6 | 34 | 526 |
| Example 5 | P13 | N6 | 32 | 532 |
| Example 6 | P16 | N8 | 36 | 531 |
| Example 7 | P20 | N6 | 34 | 630 |
| Example 8 | P25 | N6 | 35 | 625 |
| Example 9 | P30 | N8 | 33 | 625 |
| Example 10 | P34 | N15 | 32 | 630 |
| Example 11 | P37 | N16 | 36 | 632 |
| Comparative | SubPC | C₆₀ | 15 | 530 |
| Example 1 | | | | |
| Comparative | SubNC | C₆₀ | 10 | 650 |
| Example 2 | | | | |

Referring to Table 2, Examples 1 to 11 have external quantum efficiency values of 32% to 38%, and Comparative Examples 1 and 2 have external quantum efficiency values of 10% and 15%. The external quantum efficiency values of Examples 1 to 11 are at least about 2.13 times and up to 3.8 times those of Comparative Examples 1 and 2. Without being bound by any particular theory, it is believed that greater external quantum efficiency values indicate greater conversion of provided light into electrical energy, and thus it is considered that Examples 1 to 11 may each convert more light energy into electrical signals than Comparative Examples 1 and 2, and may thus be determined to have excellent or suitable charge mobility and light absorption. Examples 1 to 6 each have a maximum absorption wavelength of about 530 nm, and Examples 7 to 11 each have a maximum absorption wavelength of about 630 nm. For example, Examples 1 to 6 each may be to absorb up to the wavelength range of green visible light, and Examples 7 to 11 each may be to absorb up to the wavelength range of green visible light and red visible light. Example Compounds included in Examples 1 to 6 have no linker connecting the core and the electron accepting portion, and thus may absorb light up to the green visible light wavelength range. Without being bound by any particular theory, it is believed the linker connecting the core and the electron accepting portion may expand the absorption wavelength range by regulating the conjugation length of the entire first compound, and accordingly, Examples 7 to 11, each of which include a linker connecting the core and the electron accepting portion, may absorb visible light in the red wavelength range.

The polycyclic compound of one or more embodiments includes a three-ring fused ring, which contains two sulfur(S) atoms as ring-forming atoms, as a central structure (hereinafter referred to as core). The core of the polycyclic compound of one or more embodiments includes two thiophenes and one benzene ring. In the core of the polycyclic compound of one or more embodiments, any one carbon of the benzene ring is named benzene carbon 1, and carbons are sequentially named benzene carbon 2 to 6 in a counterclockwise direction with respect to the benzene carbon, and carbons are sequentially named thiophene carbons 1 to 4 in counterclockwise direction with respect to the sulfur atom of thiophene. Benzene carbon 2 and benzene carbon 3 of the benzene ring are each bonded to be identical to thiophene carbon 2 and thiophene carbon 1 of any one thiophene, and benzene carbon 5 and benzene carbon 6 of the benzene ring are each bonded to be identical to thiophene carbon 2 and thiophene carbon 1 of another thiophene. In particular, in one or more embodiments, hydrogen atoms having a relatively small volume are substituted on benzene carbon 1 and benzene carbon 4 of the benzene ring of the core, and thus the core may maintain a planar structure, improving the charge mobility of the polycyclic compound and improving light absorption. In one or more embodiments, the deposition stability and heat resistance of the polycyclic compound are excellent or suitable, and accordingly, a thin film has excellent or suitable purity upon continuous deposition.

It is seen that the compounds included in each of Comparative Examples 1 and 2 are not provided with the characteristics of one or more embodiments of the present disclosure, and therefore have lower charge mobility and lower light conversion efficiency than that of the compounds of Examples when applied to elements.

A light receiving element of one or more embodiments may exhibit element characteristics such as relatively high efficiency and improved deposition quality.

An electronic device of one or more embodiments may exhibit improved light reception efficiency.

A polycyclic compound of one or more embodiments may be included in a light receiving layer of a light receiving element and may thus contribute to improving the efficiency of the light receiving element and an electronic device.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this disclosure, including the claims, to expressly recite any sub-range subsumed within the ranges expressly recited herein.

The light-emitting element, the light receiving element, the electronic devices/apparatus, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the present disclosure has been described with reference to one or more embodiments of the present disclosure, it will be understood that the present disclosure should not be limited to these embodiments but one or more suitable changes and modifications may be made by those skilled in the art without departing from the spirit and scope of the present disclosure.

Accordingly, the technical scope of the present disclosure is not intended to be limited to the contents set forth in the detailed description of the disclosure, but is intended to be defined by the appended claims and equivalents thereof.

What is claimed is:

1. A light receiving element comprising:
a first electrode;
a second electrode facing the first electrode; and
a light receiving layer between the first electrode and the second electrode and comprising a first compound represented by Formula 1: 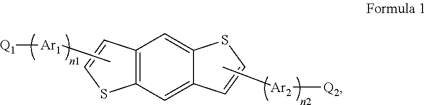
wherein, in Formula 1,
n₁ and n₂ are each independently an integer of 0 to 10,
Ar₁ and Ar₂ are each independently a substituted or unsubstituted arylene group having 6 to 60 carbon atoms or a substituted or unsubstituted heteroarylene group having 2 to 60 carbon atoms, and
Q₁ and Q₂ are each independently represented by any one selected from among Formulas Q-1 to Q-7: 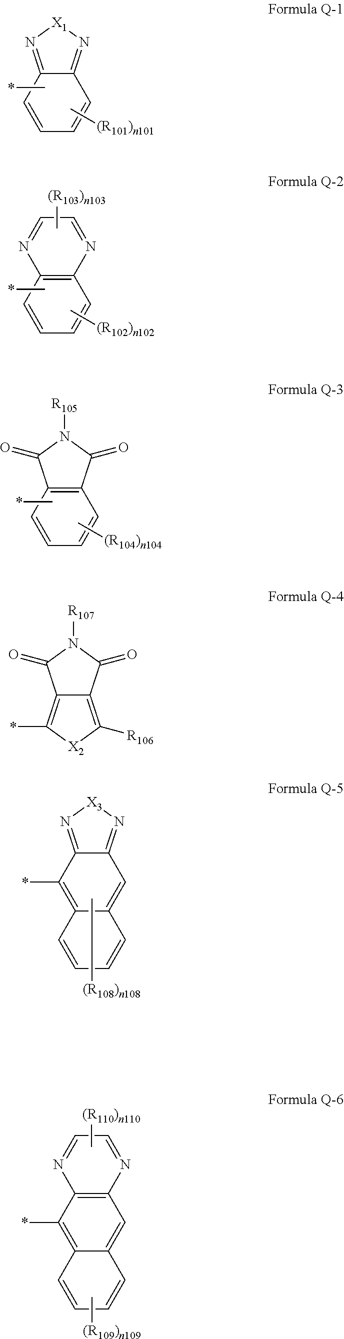 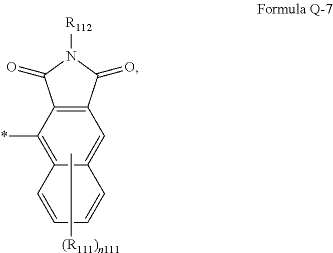
in Formulas Q-1 to Q-7,
n₁₀₁, n₁₀₂, and n₁₀₄ being each independently an integer of 0 to 3,
n₁₀₃ and n₁₁₀ being each independently an integer of 0 to 2,
n₁₀₈, n₁₀₉, and n₁₁₁ being each independently an integer of 0 to 5,
X₁ to X₃ being each independently oxygen, sulfur, selenium, or tellurium,
R₁₀₁ to R₁₀₃ being each independently hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms,
R₁₀₄ to R₁₁₂ being each independently hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms, and
-* being a site linked to Formula 1.

2. The light receiving element of claim 1, wherein, in Formula 1,
when Q₁ and Q₂ are each represented by Formula Q-1, then Q₁ and Q₂ are each independently represented by any one selected from among Formulas R-11 to R-19: 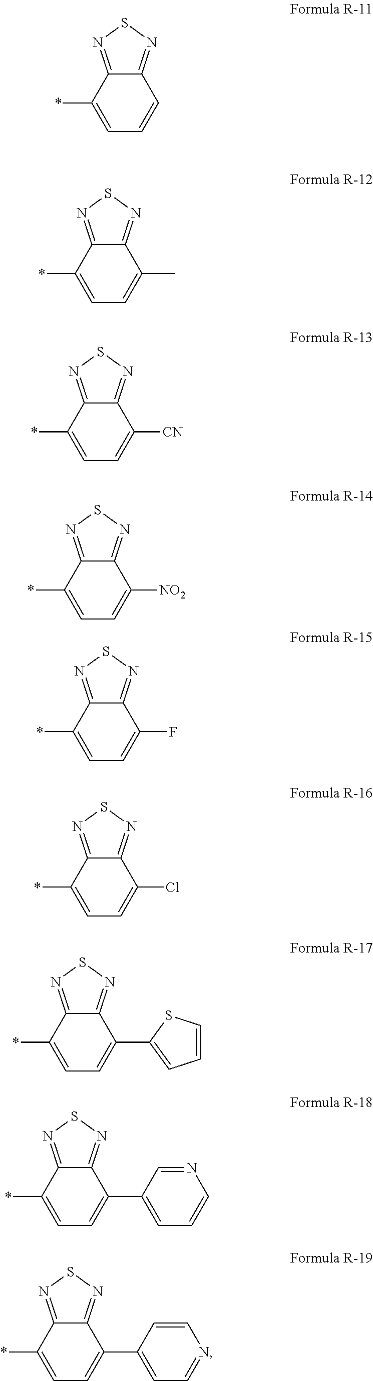
in Formulas R-11 to R-19,
-* being a site linked to Formula 1.

3. The light receiving element of claim 1, wherein, in Formula 1,
when Q₁ and Q₂ are each represented by Formula Q-2, then Q₁ and Q₂ are each independently represented by any one selected from among Formulas R-21 to R-24: 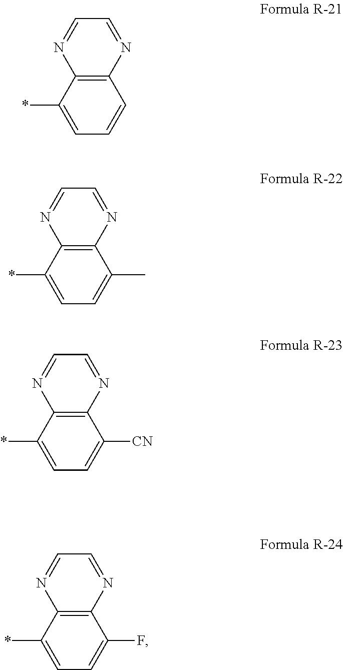 [SMILES: *c1cccc2nccnc12 ; *c1ccc(C)c2nccnc12 ; *c1ccc(C#N)c2nccnc12 ; *c1ccc(F)c2nccnc12]
in Formulas R-21 to R-24,
-* being a site linked to Formula 1.

4. The light receiving element of claim 1, wherein, in Formula 1,
when Q₁ and Q₂ are each represented by Formula Q-3, then Q₁ and Q₂ are each independently represented by any one selected from among Formulas R-31 to R-34: 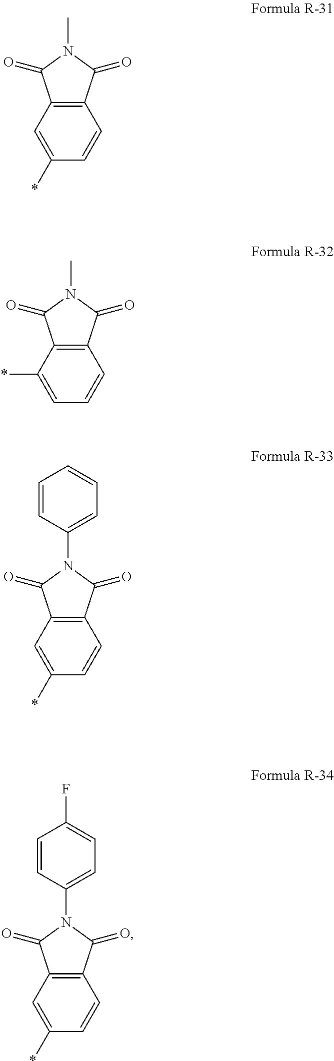 [SMILES: *c1ccc2c(c1)C(=O)N(C)C2=O ; *c1cccc2c1C(=O)N(C)C2=O ; *c1ccc2c(c1)C(=O)N(c1ccccc1)C2=O ; *c1ccc2c(c1)C(=O)N(c1ccc(F)cc1)C2=O]
in Formulas R-31 to R-34,
-* being a site linked to Formula 1.

5. The light receiving element of claim 1, wherein, in Formula 1,
when Q₁ and Q₂ are each represented by Formula Q-4, then Q₁ and Q₂ are each independently represented by Formula R-41 or Formula R-42: 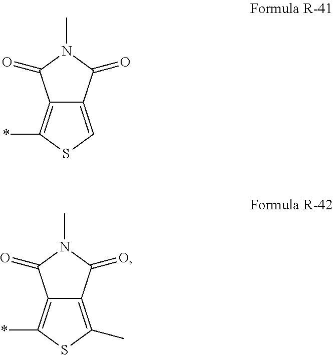 [SMILES: *c1scc2c1C(=O)N(C)C2=O ; *c1sc(C)c2c1C(=O)N(C)C2=O]
in Formulas R-41 and R-42,
-* being a site linked to Formula 1.

6. The light receiving element of claim 1, wherein, in Formula 1,
when Q₁ and Q₂ are each represented by Formula Q-5, then Q₁ and Q₂ are each independently represented by Formula R-51 or Formula R-52: 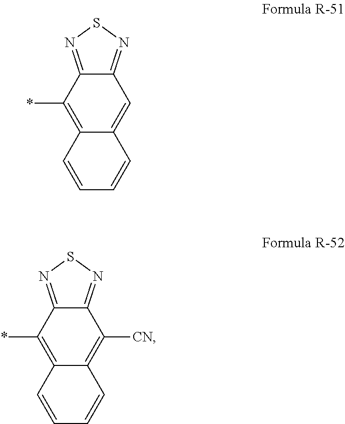 [SMILES: *c1c2ccccc2cc2nsnc12 ; *c1c2ccccc2c(C#N)c2nsnc12]
in Formulas R-51 and R-52,
-* being a site linked to Formula 1.

7. The light receiving element of claim 1, wherein, in Formula 1,
when Q₁ and Q₂ are each represented by Formula Q-6, then Q₁ and Q₂ are each independently represented by Formula R-61, and
when Q₁ and Q₂ are each represented by Formula Q-7, then Q₁ and Q₂ are each independently represented by Formula R-71: 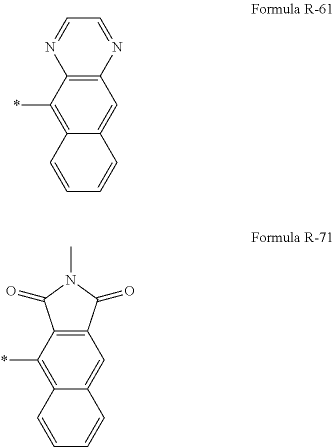 [SMILES: *c1c2ccccc2cc2nccnc12 ; *c1c2c(cc3ccccc13)C(=O)N(C)C2=O]
in Formulas R-61 and R-71,
-* being a site linked to Formula 1.

8. The light receiving element of claim 1, wherein the first compound represented by Formula 1 is represented by any one selected from among Formulas A-1 to A-5: 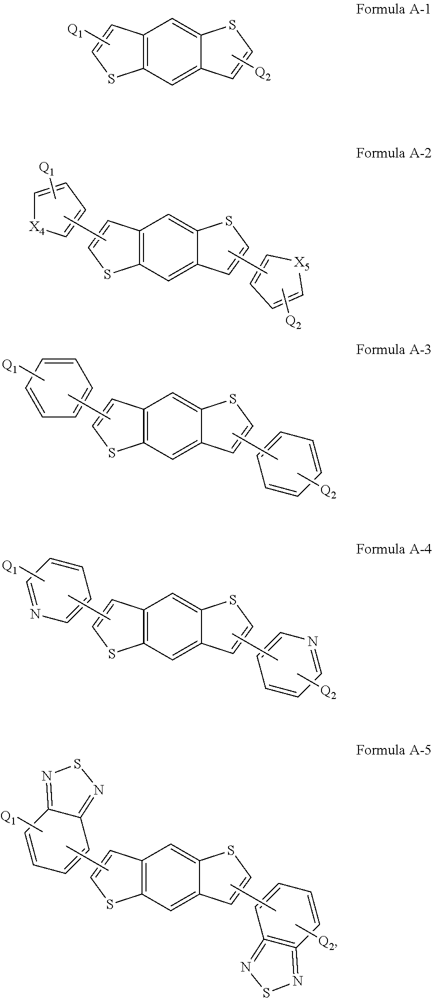
in Formulas A-1 to A-5,
X₄ and X₅ being each independently oxygen, sulfur, selenium, or tellurium, and
Q₁ and Q₂ each being the same as defined in Formula 1.

9. The light receiving element of claim 1, wherein the first compound represented by Formula 1 is represented by any one selected from among Formulas 1-1 to 1-6: 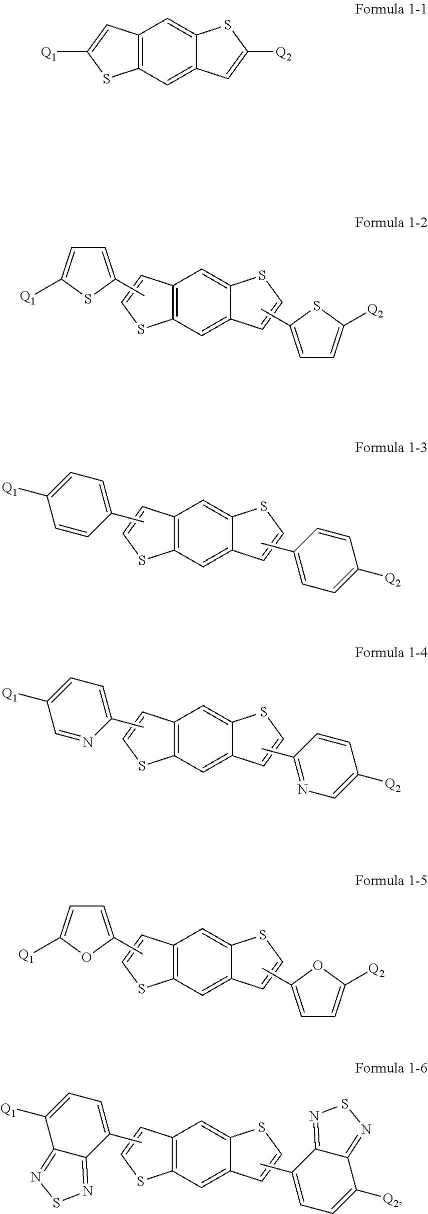
in Formulas 1-1 to 1-6,
Q₁ and Q₂ each being the same as defined in Formula 1.

10. The light receiving element of claim 1, wherein the light receiving layer further comprises a second compound represented by Formula 2: 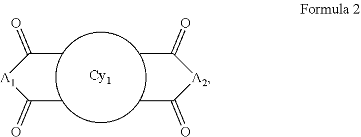
in Formula 2,
Cy₁ is being substituted or unsubstituted aromatic hydrocarbon ring having 6 to 30 carbon atoms,
A₁ and A₂ being each independently oxygen or NR113, and
R₁₁₃ being hydrogen, deuterium, a halogen, a substituted or unsubstituted alkyl group having 1 to 30 carbon atoms, a substituted or unsubstituted aryl group having 6 to 30 carbon atoms, a substituted or unsubstituted heteroaryl group having 2 to 30 carbon atoms, or a substituted or unsubstituted cycloalkyl group having 3 to 30 carbon atoms.

11. The light receiving element of claim 10, wherein the light receiving layer comprises a light absorption layer comprising the first compound and a photoelectric conversion layer comprising the second compound.

12. The light receiving element of claim 11, wherein the photoelectric conversion layer is on the light absorption layer.

13. The light receiving element of claim 11, wherein an interface is defined between the light absorption layer and the photoelectric conversion layer.

14. The light receiving element of claim 1, further comprising:
a hole control layer between the first electrode and the light receiving layer; and
an electron control layer between the light receiving layer and the second electrode.

15. The light receiving element of claim 10, wherein the first compound represented by Formula 1 is represented by any one selected from among compounds of Compound Group 1, and the second compound represented by Formula 2 is represented by any one selected from among compounds of Compound Group 2:

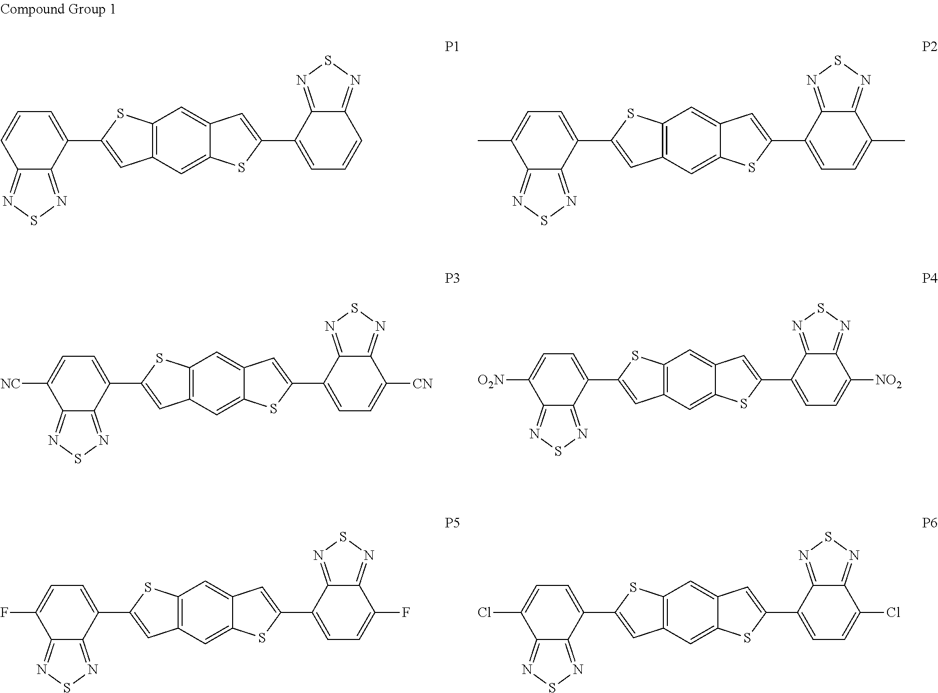

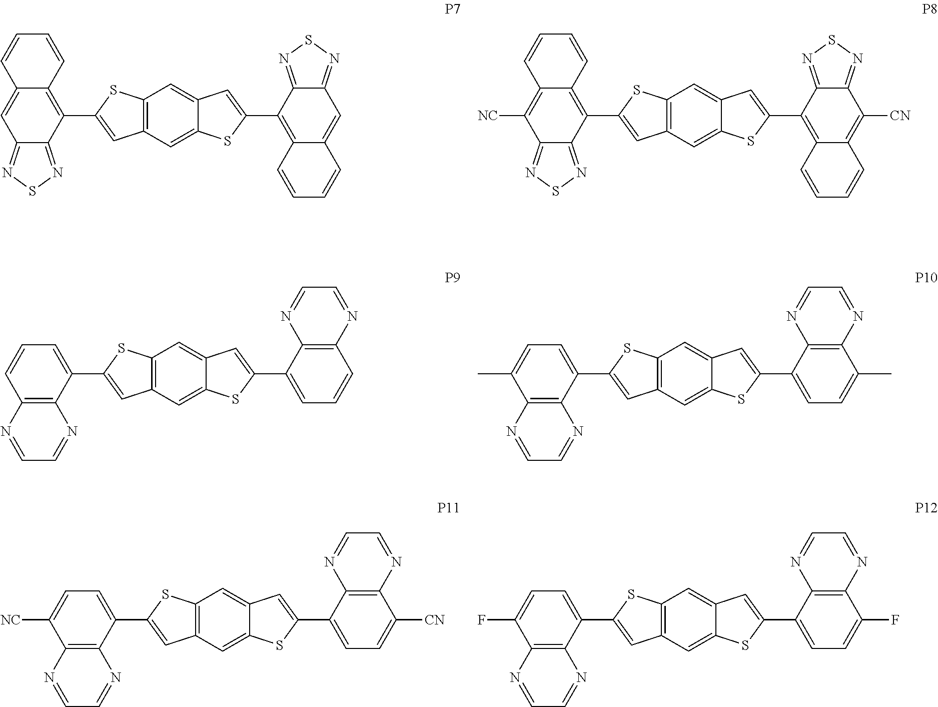

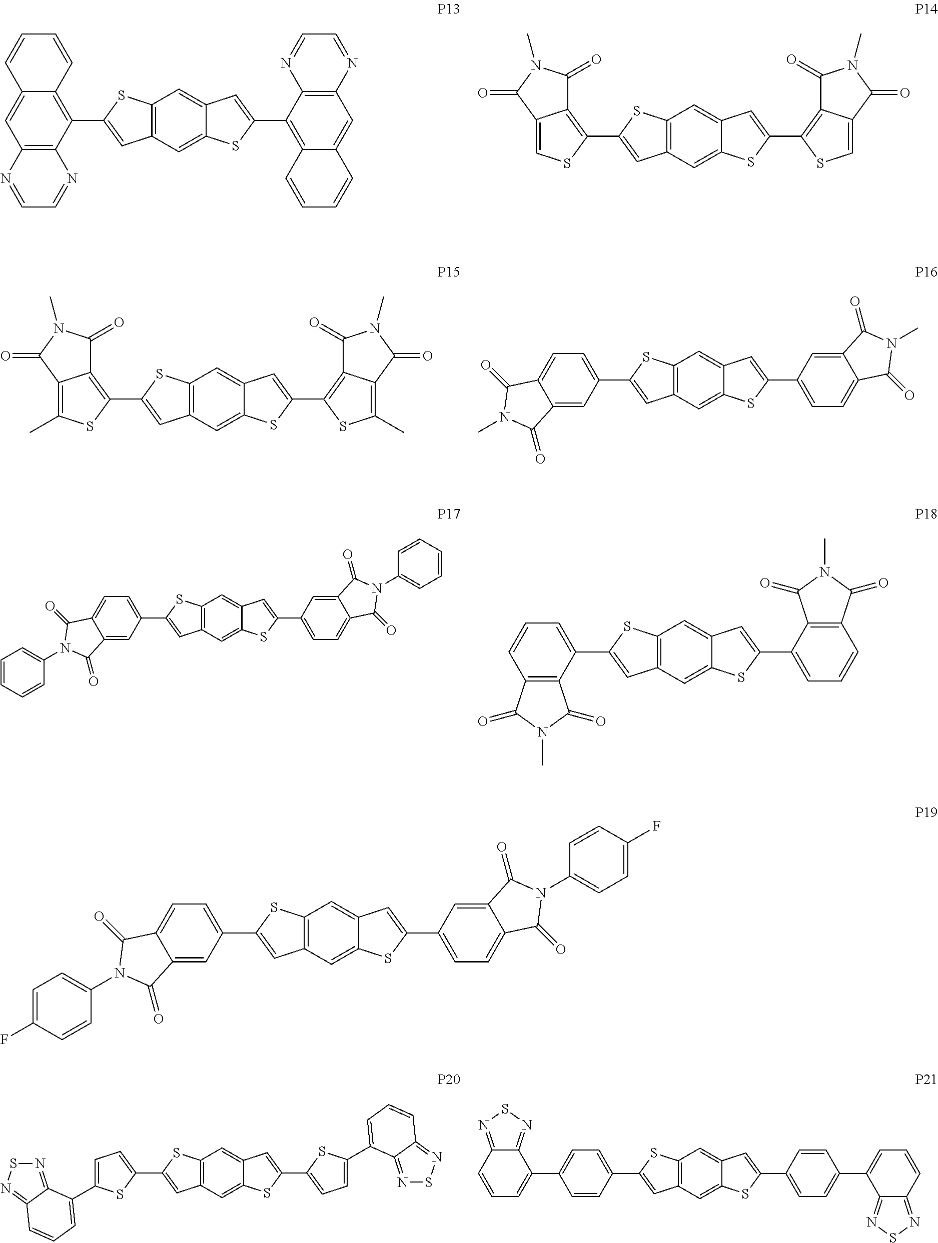

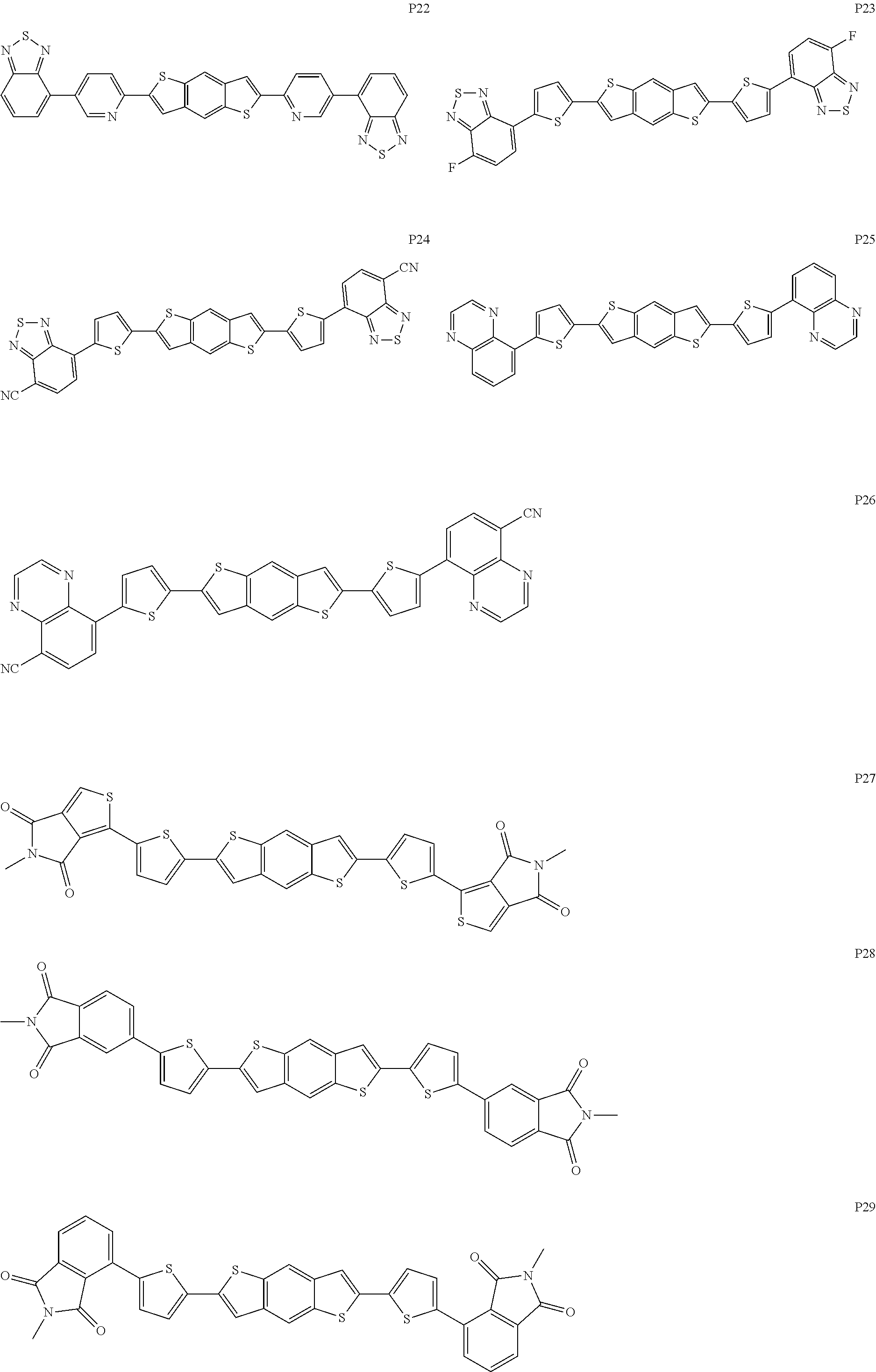

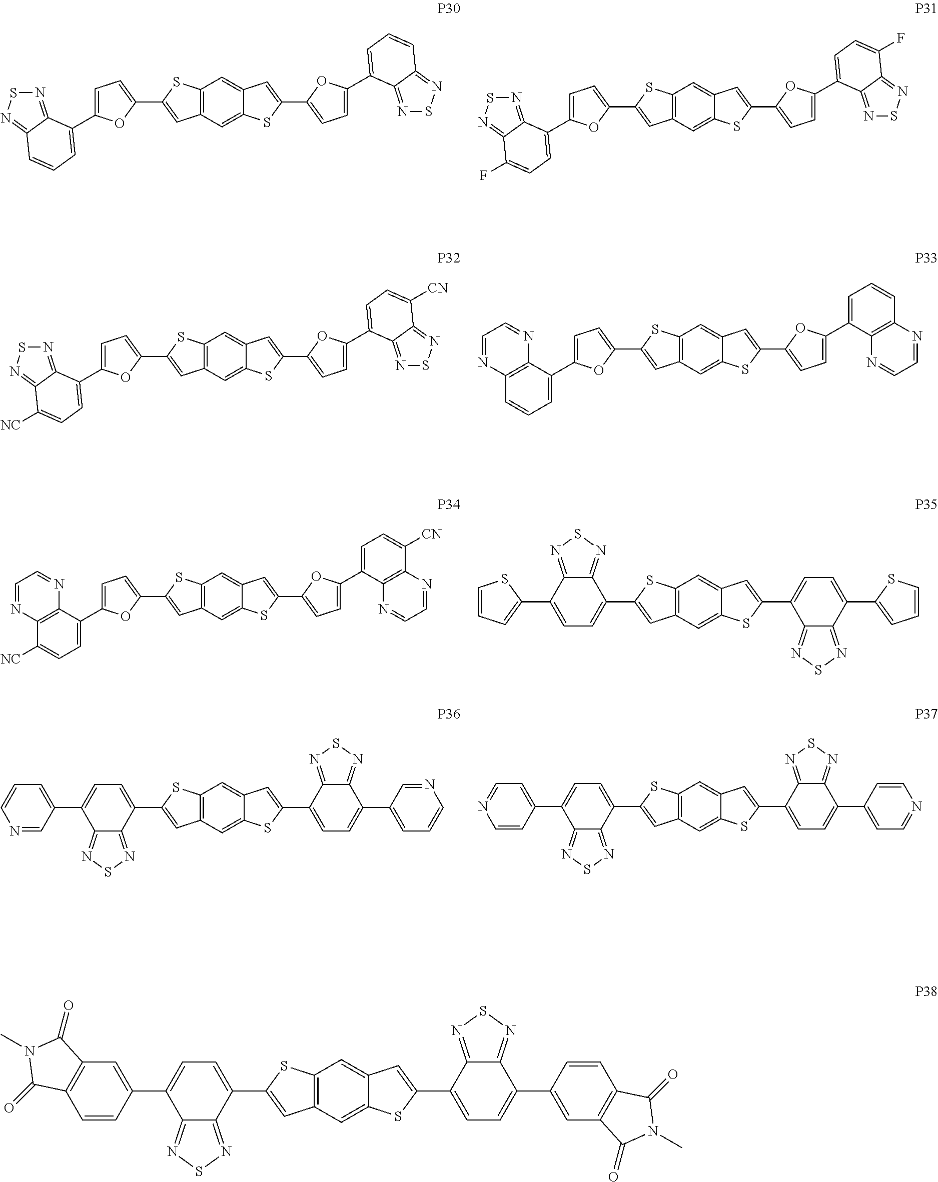

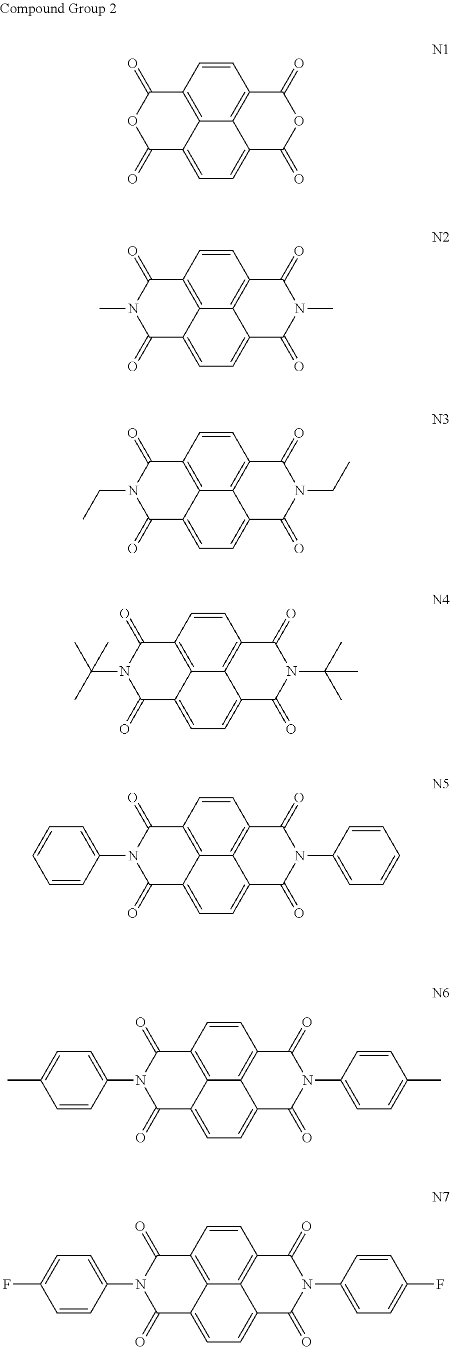

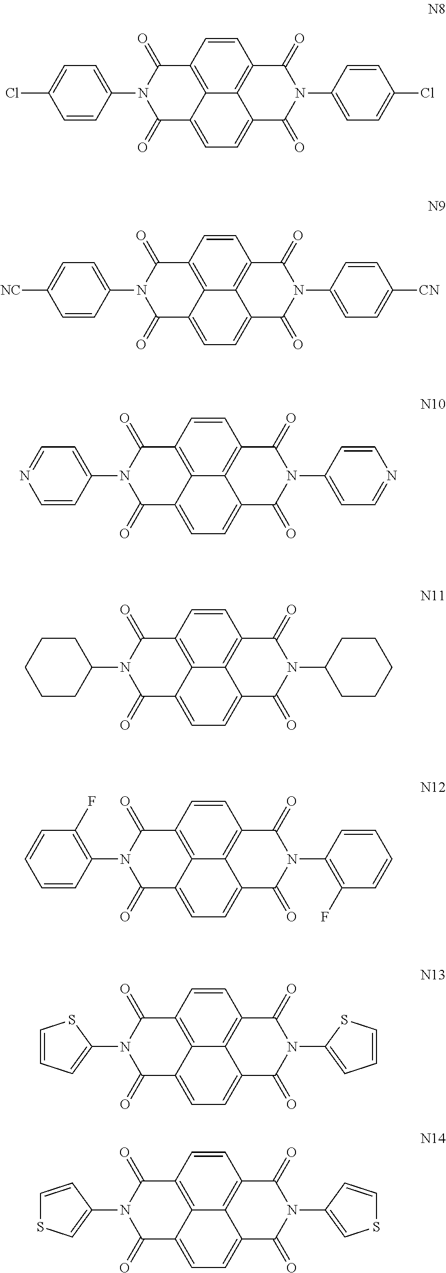

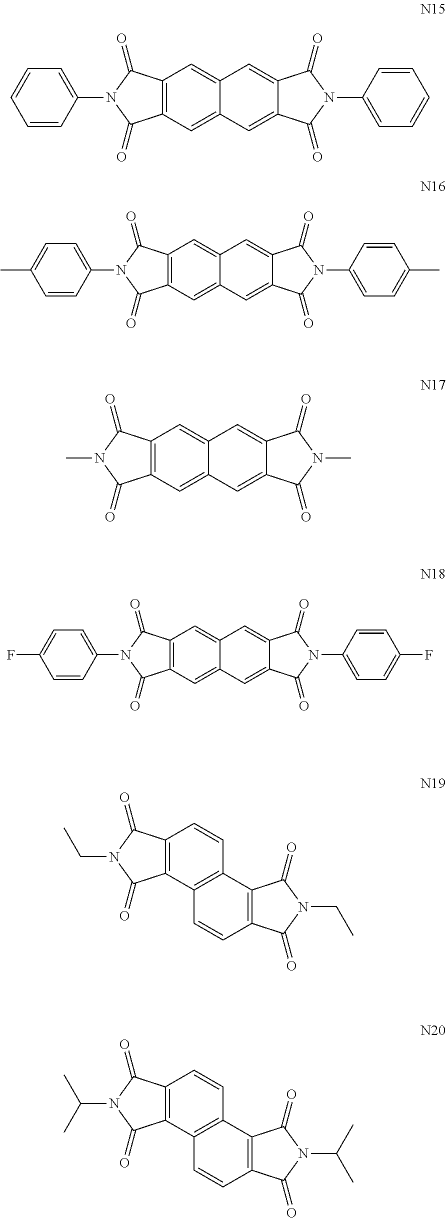

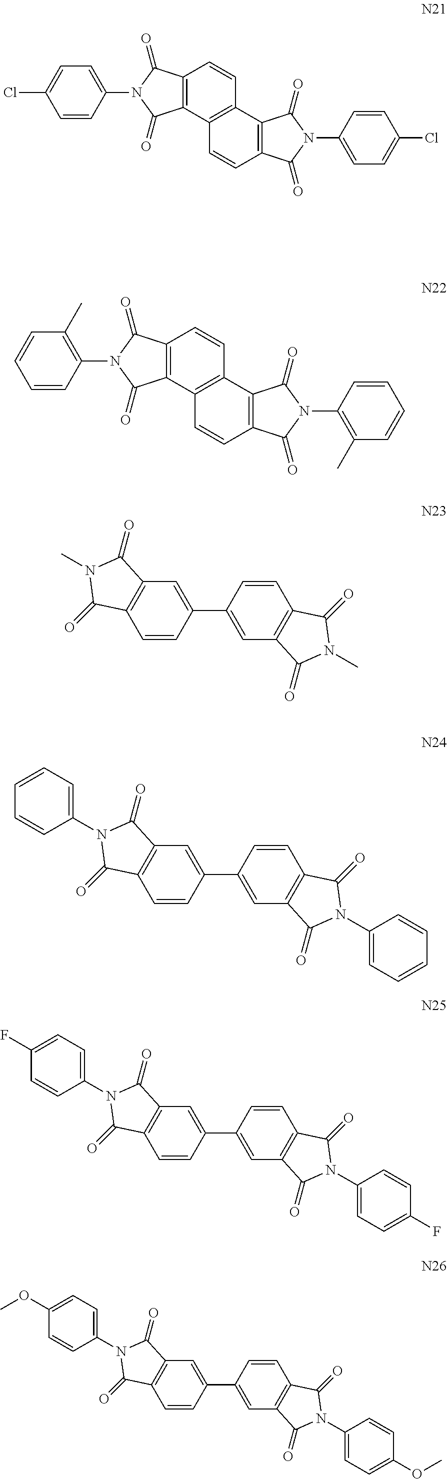

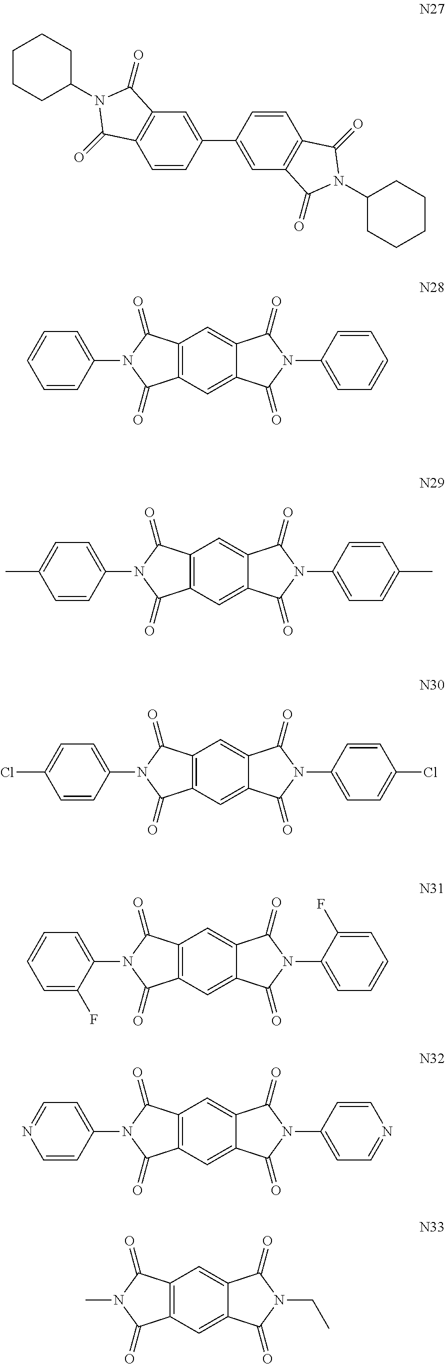

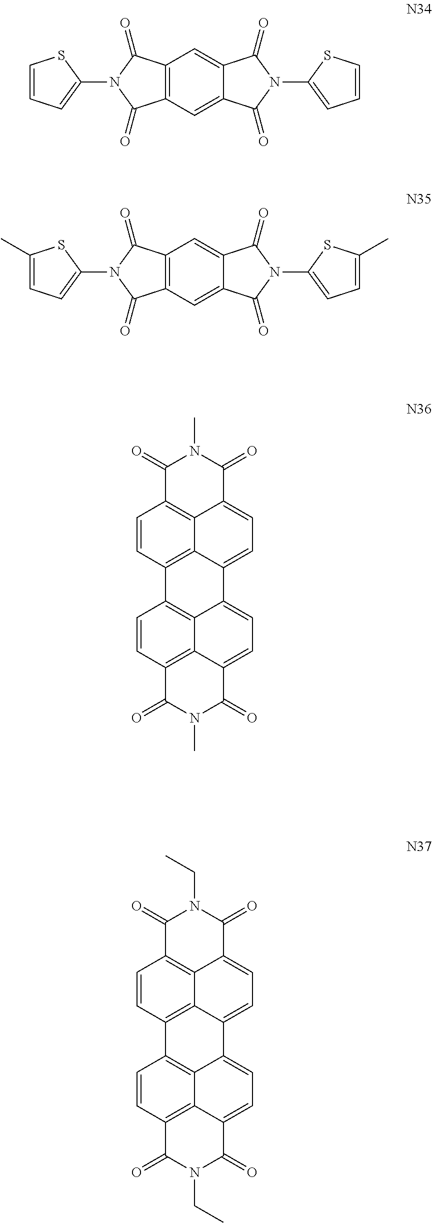

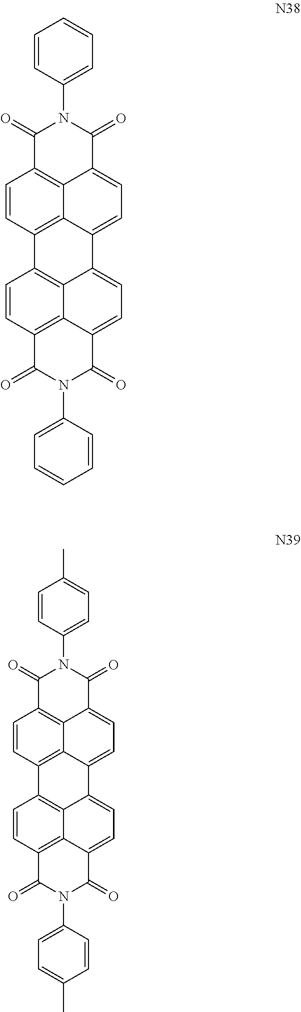 [SMILES: O=C1c2ccc3c4ccc5c6c(ccc(c7ccc(c2c37)C(=O)N1c1ccccc1)c64)C(=O)N(c1ccccc1)C5=O ; Cc1ccc(N2C(=O)c3ccc4c5ccc6c7c(ccc(c8ccc(c3c48)C2=O)c75)C(=O)N(c2ccc(C)cc2)C6=O)cc1]

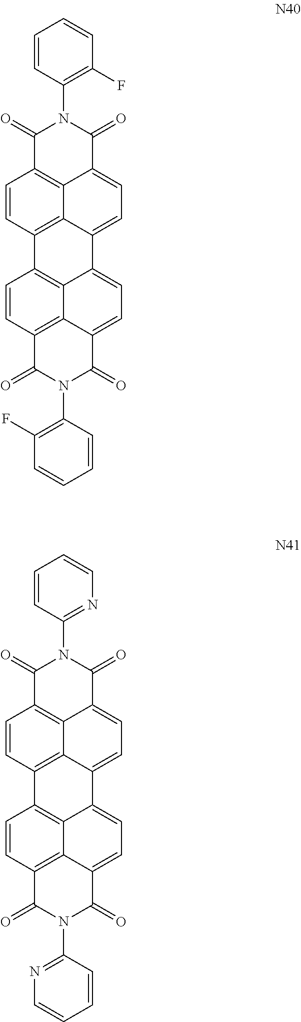 [SMILES: O=C1c2ccc3c4ccc5c6c(ccc(c7ccc(c2c37)C(=O)N1c1ccccc1F)c64)C(=O)N(c1ccccc1F)C5=O ; O=C1c2ccc3c4ccc5c6c(ccc(c7ccc(c2c37)C(=O)N1c1ccccn1)c64)C(=O)N(c1ccccn1)C5=O]

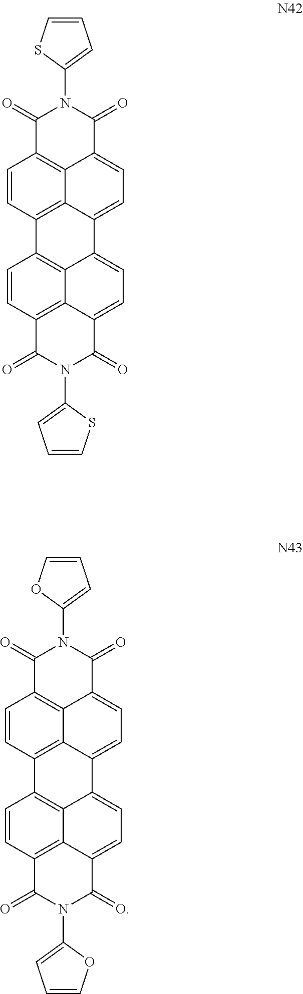 [SMILES: O=C1c2ccc3c4ccc5c6c(ccc(c7ccc(c2c37)C(=O)N1c1cccs1)c64)C(=O)N(c1cccs1)C5=O ; O=C1c2ccc3c4ccc5c6c(ccc(c7ccc(c2c37)C(=O)N1c1ccco1)c64)C(=O)N(c1ccco1)C5=O]

16. An electronic device comprising:
a base layer; and
a display element layer comprising a light emitting element and a light receiving element,
wherein:
the light emitting element comprises:
a first light emitting electrode on the base layer;
an emission layer on the first light emitting electrode; and
a second light emitting electrode on the emission layer,
the light receiving element comprises:
a first light receiving electrode on the base layer;
a light receiving layer on the first light receiving electrode; and
a second light receiving electrode on the light receiving layer, and
the light receiving layer comprises a first compound represented by Formula 1: 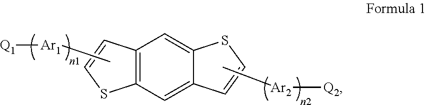
wherein, in Formula 1,
n₁ and n₂ are each independently an integer of 0 to 10,
Ar₁ and Ar₂ are each independently a substituted or unsubstituted arylene group having 6 to 60 carbon atoms or a substituted or unsubstituted heteroarylene group having 2 to 60 carbon atoms, and
Q₁ and Q₂ are each independently represented by any one selected from among Formulas Q-1 to Q-7: 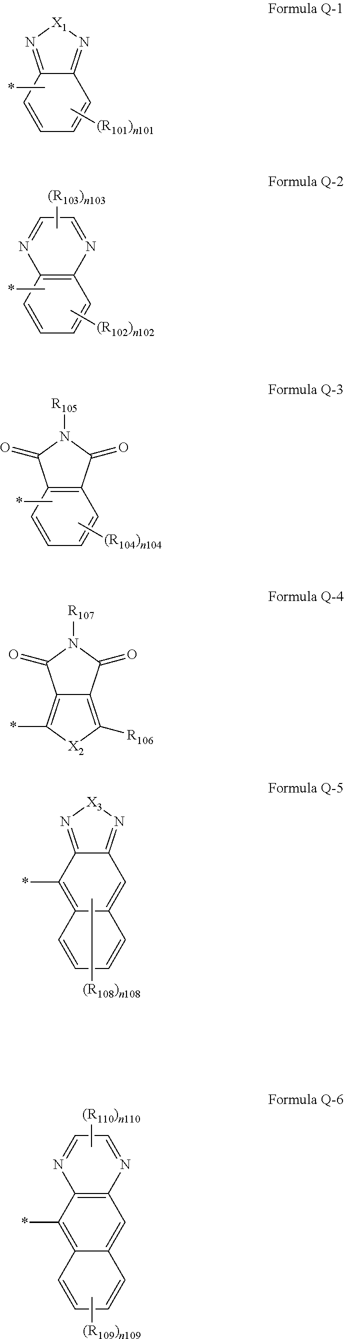 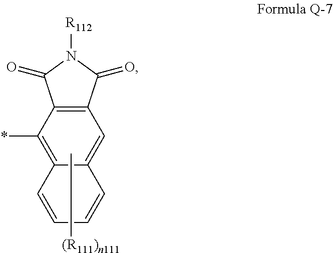
in Formulas Q-1 to Q-7,
n₁₀₁, n₁₀₂, and n₁₀₄ being each independently an integer of 0 to 3,
n₁₀₃ and n₁₁₀ being each independently an integer of 0 to 2,
n₁₀₈, n₁₀₉, and n₁₁₁ being each independently an integer of 0 to 5,
X₁ to X₃ being each independently oxygen, sulfur, selenium, or tellurium,
R₁₀₁ to R₁₀₃ being each independently hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms,
R₁₀₄ to R₁₁₂ being each independently hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms, and
-* being a site linked to Formula 1.

17. A polycyclic compound represented by Formula 1: 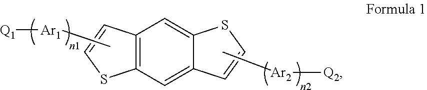
wherein, in Formula 1,
n₁ and n₂ are each independently an integer of 0 to 10,
Ar₁ and Ar₂ are each independently a substituted or unsubstituted arylene group having 6 to 60 carbon atoms or a substituted or unsubstituted heteroarylene group having 2 to 60 carbon atoms, and
Q₁ and Q₂ are each independently represented by any one selected from among Formulas Q-1 to Q-7: 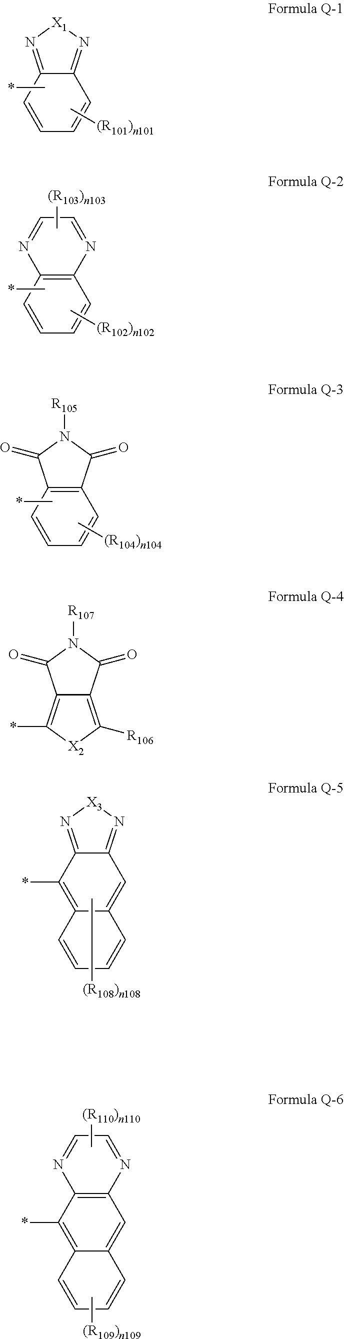 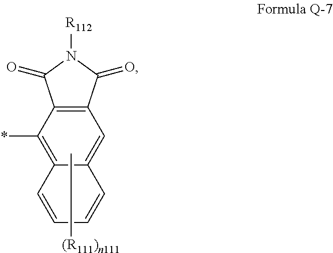
in Formulas Q-1 to Q-7,
n₁₀₁, n₁₀₂, and n₁₀₄ being each independently an integer of 0 to 3,
n₁₀₃ and n₁₁₀ being each independently an integer of 0 to 2,
n₁₀₈, n₁₀₉, and n₁₁₁ being each independently an integer of 0 to 5,
X₁ to X₃ being each independently oxygen, sulfur, selenium, or tellurium,
R₁₀₁ to R₁₀₃ being each independently hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms,
R₁₀₄ to R₁₁₂ being each independently hydrogen, deuterium, a halogen, a cyano group, a nitro group, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms, and
-* being a site linked to Formula 1.

18. The polycyclic compound of claim 17, wherein the polycyclic compound represented by Formula 1 is represented by any one selected from among Formulas A-1 to A-5: 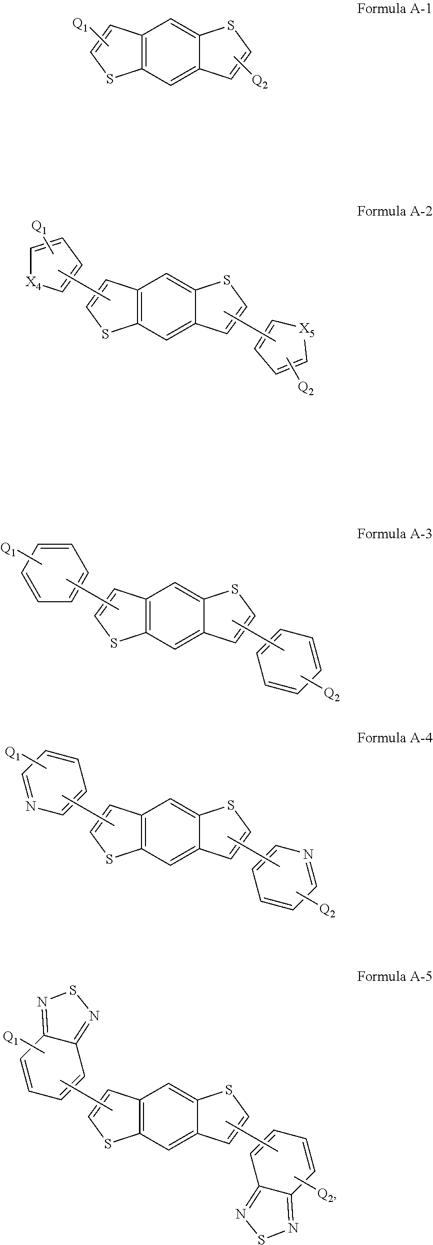
in Formulas A-1 to A-5,
X₄ and X₅ being each independently oxygen, sulfur, selenium, or tellurium, and
Q₁ and Q₂ each being the same as defined in Formula 1.

19. The polycyclic compound of claim 17, wherein the polycyclic compound represented by Formula 1 is represented by any one selected from among Formulas 1-1 to 1-6: 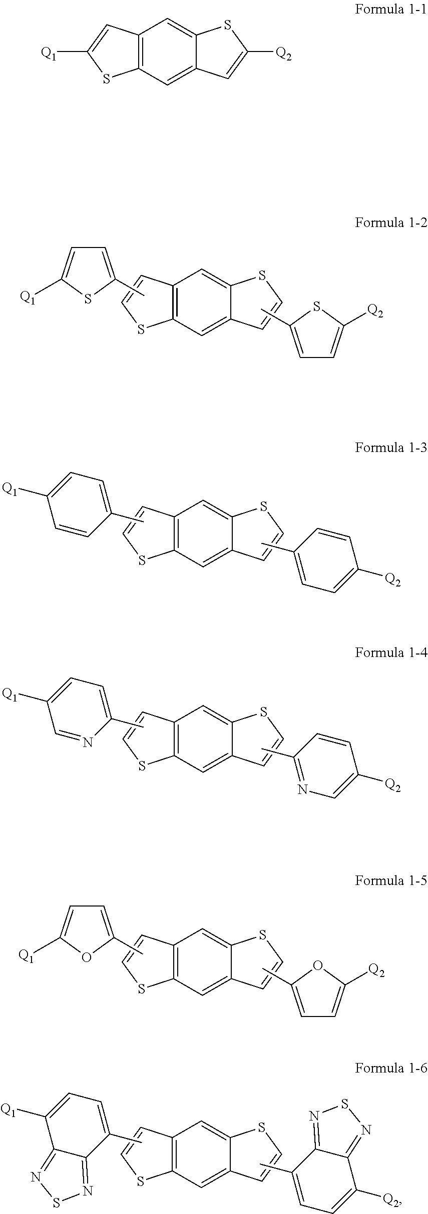
in Formulas 1-1 to 1-6,
Q₁ and Q₂ each being the same as defined in Formula 1.

20. The polycyclic compound of claim 17, wherein the polycyclic compound represented by Formula 1 is represented by any one selected from among compounds of Compound Group 1: 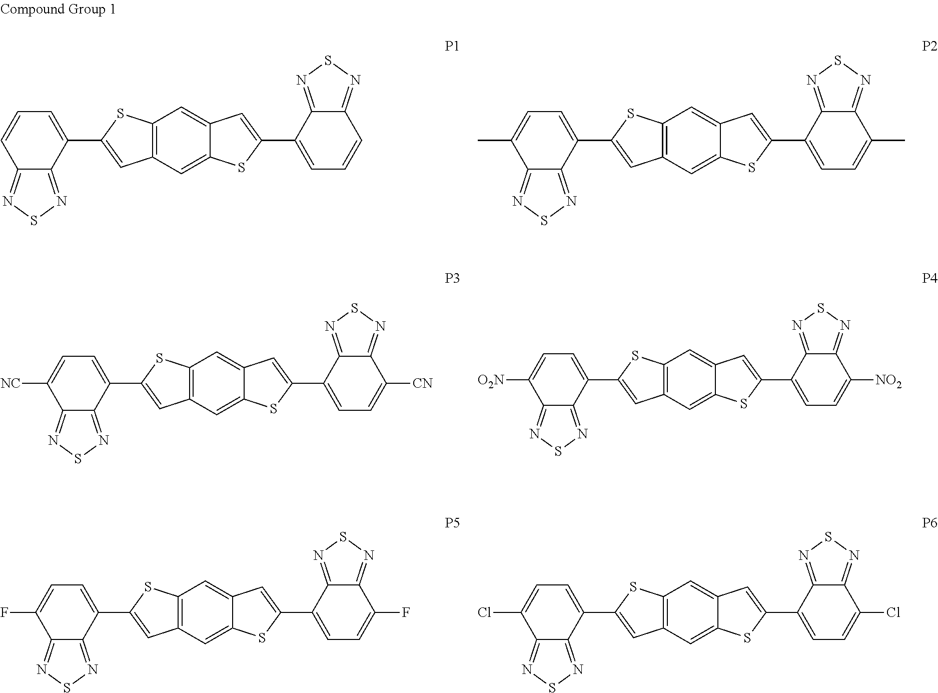 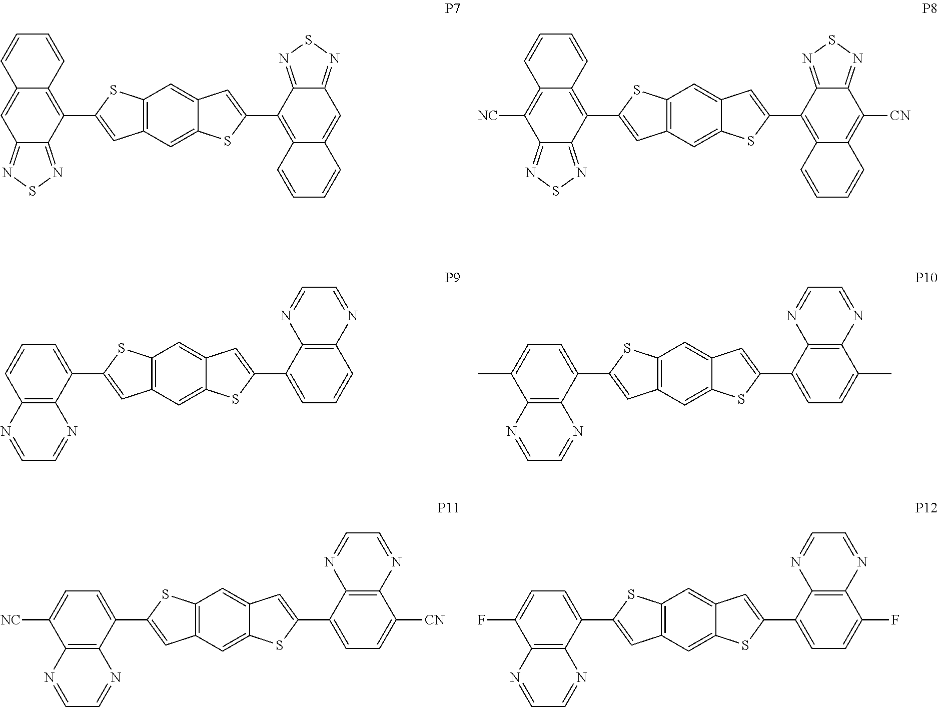 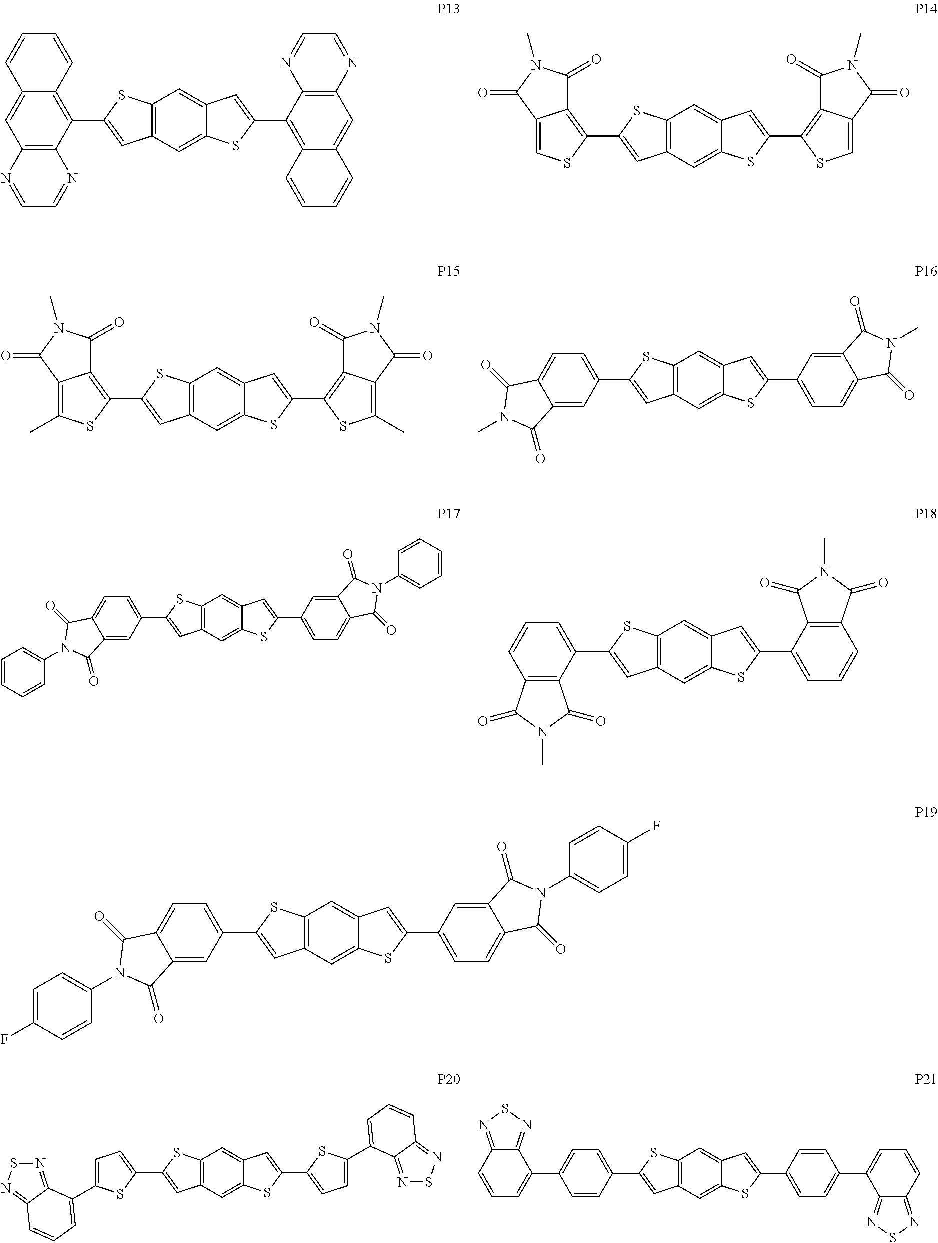 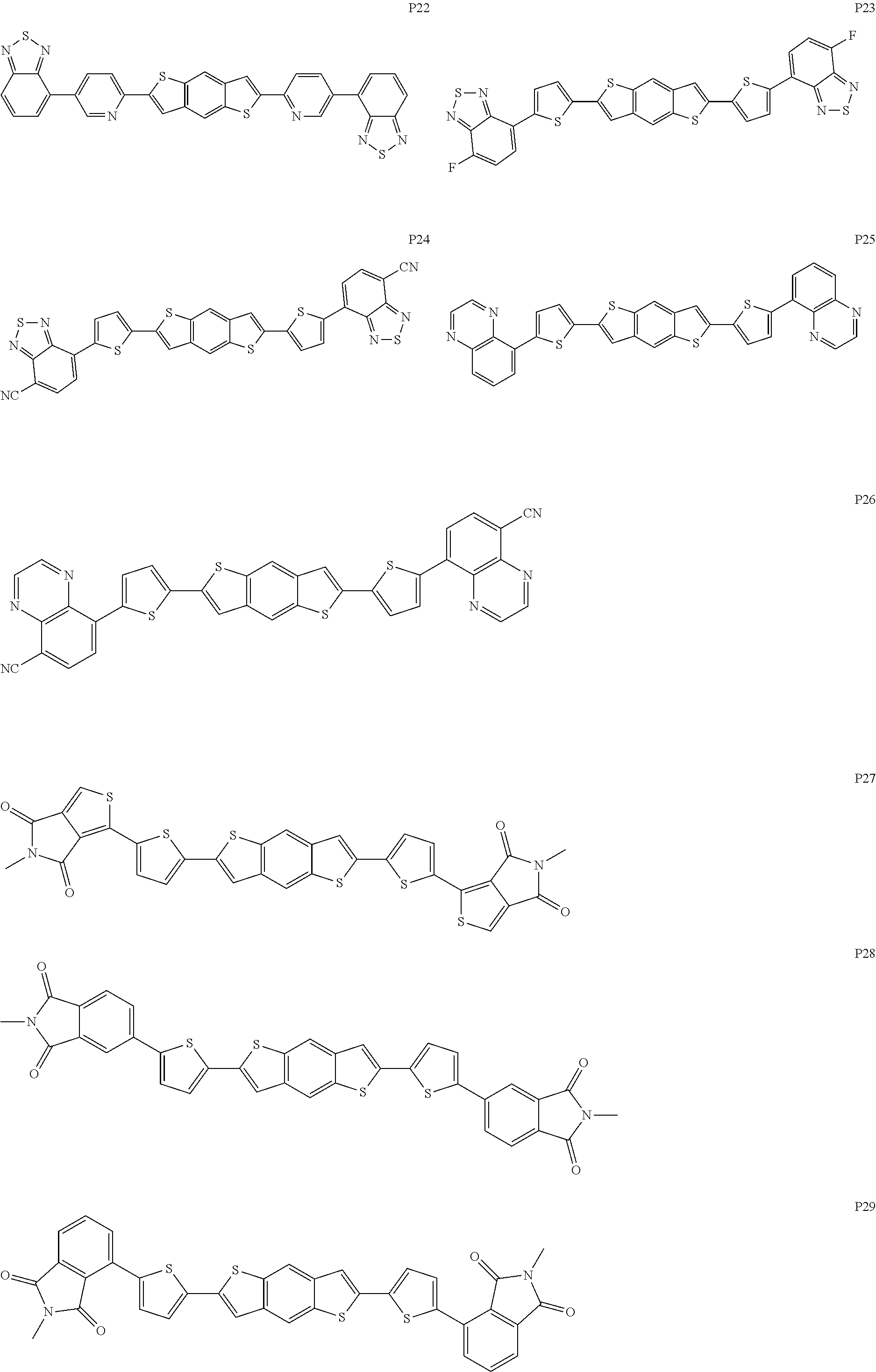 [chemical structure]
